SEQUENCE LISTING (7 sequences)

SEQ ID NO: 1 - DNA, 21 bp, synthetic construct
  misc_feature 1..21: note = Description of Artificial Sequence: Syntheticoligonucleotide
  source 1..21: mol_type = other DNA
tagagggtgatcttgaagtac

SEQ ID NO: 2 - DNA, 21 bp, synthetic construct
  misc_feature 1..21: note = Description of Artificial Sequence: Syntheticoligonucleotide
  source 1..21: mol_type = other DNA
aagaacaccatagcatgctgg

SEQ ID NO: 3 - DNA, 21 bp, synthetic construct
  misc_feature 1..21: note = Description of Artificial Sequence: Syntheticoligonucleotide
  source 1..21: mol_type = other DNA
aagttctgagttgagtcctgg

SEQ ID NO: 4 - DNA, 21 bp, synthetic construct
  misc_feature 1..21: note = Description of Artificial Sequence: Syntheticoligonucleotide
  source 1..21: mol_type = other DNA
tatttcccagagtgaactggc

SEQ ID NO: 5 - DNA, 21 bp, synthetic construct
  misc_feature 1..21: note = Description of Artificial Sequence: Syntheticoligonucleotide
  source 1..21: mol_type = other DNA
aagtgaatgcaaagcagcagg

SEQ ID NO: 6 - DNA, 21 bp, synthetic construct
  misc_feature 1..21: note = Description of Artificial Sequence: Syntheticoligonucleotide
  source 1..21: mol_type = other DNA
aatctgccgcttcactatcag

SEQ ID NO: 7 - DNA, 76 bp, synthetic construct
  misc_feature 1..76: note = Description of Artificial Sequence: Syntheticoligonucleotide
  source 1..76: mol_type = other DNA
gtgactacgtgctgcctaggtgactacgtgctgcctaggtgactacgtgctgcctaggtgactacgtgctgcctag

(19) United States
(12) Patent Application Publication
(10) Pub. No.: US 2024/0050385 A1
(43) Pub. Date: Feb. 15, 2024

(54) METHODS OF TREATING LEPTOMENINGEAL METASTASIS

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Adrienne Boire, New York, NY (US); Yudan Chi, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 18/155,115

(22) Filed: Jan. 17, 2023

Related U.S. Application Data
(63) Continuation of application No. PCT/US2021/041828, filed on Jul. 15, 2021.
(60) Provisional application No. 63/052,139, filed on Jul. 15, 2020.

(51) Int. Cl. A61K 31/16; A61P 35/04; A61K 9/00

(52) U.S. Cl. CPC A61K 31/16; A61P 35/04; A61K 9/0019; A61K 2121/00

(57) ABSTRACT

The present disclosure relates to the use of iron chelation to prevent and/or treat leptomeningeal metastasis. In certain embodiments, the present disclosure provides methods for the prevention and/or treatment of leptomeningeal metastasis that include the administration of an iron chelator to a subject. The present disclosure further provides kits for performing such methods.

### CROSS-REFERENCE TO RELATED APPLICATIONS

This application is a continuation of International Application No. PCT/US2021/041828, filed Jul. 15, 2021, which claims priority to U.S. Provisional Application No. 63/052,139, filed Jul. 15, 2020, the contents of each of which are incorporated by reference in their entireties, and to which priority is claimed.

### SEQUENCE LISTING

The specification further incorporates by reference the Sequence Listing submitted herewith via EFS on Jan. 17, 2023. Pursuant to 37 C.F.R. § 1.52(e)(5), the Sequence Listing xml file, identified as 072734 1432.xml, is 10,227 bytes and was created on Jan. 17, 2023. The Sequence Listing, electronically filed herewith, does not extend beyond the scope of the specification and thus does not contain new matter.

### INTRODUCTION

The present disclosure relates to the use of iron chelation to prevent and/or treat leptomeningeal metastasis.

### BACKGROUND

Spread of cancer cells into the cerebrospinal fluid-filled leptomeninges is known as leptomeningeal metastasis. This form of metastasis has become increasingly common (Kesari et al., Leptomeningeal metastases. Neurol Clin 21, 25-66 (2003)) and is typically fatal within months (Oechsle et al., Prognostic factors and treatment options in patients with leptomeningeal metastases of different primary tumors: a retrospective analysis. J Cancer Res Clin Oncol 136, 1729-1735 (2010)). Under normal physiological conditions, the leptomeningeal space is isolated from the systemic circulation by the blood-cerebrospinal fluid-barrier. This anatomic compartment is hypoxic and contains sparse amounts of metabolic intermediates and micronutrients (Spector et al., A balanced view of the cerebrospinal fluid composition and functions: Focus on adult humans. Exp Neurol 273, 57-68 (2015)). In the setting of leptomeningeal metastasis, the normally acellular cerebrospinal fluid contains cancer cells as well as lymphocytes, macrophages and neutrophils. Cancer cells within this microenvironment must therefore cope with oppressive metabolic constraints while evading immune responses.

The use of CNS irradiation and targeted therapies, when available, have resulted in prolongation in overall survival in patients with leptomeningeal metastases. However, once these agents have been exhausted, the efficacy of conventional chemotherapy and intrathecal agents to control intracranial disease is quite limited, and the immune checkpoint blockade has little evidence for the treatment of leptomeningeal metastases. Accordingly, there is a need for novel agents in the treatment of leptomeningeal metastases.

### SUMMARY

The present disclosure provides methods for treating leptomeningeal metastasis in a subject. In certain embodiments, the method includes administering a therapeutically effective amount of an iron chelator to the subject. In certain embodiments, the subject has cancer. For example, but not by way of limitation, the cancer is breast cancer or lung cancer. In certain embodiments, the lung cancer is non-small cell lung carcinoma. In certain embodiments, the method further includes diagnosing the subject with leptomeningeal metastasis.

The present disclosure further provides methods for preventing and/or reducing the risk of leptomeningeal metastasis in a subject having cancer. In certain embodiments, the method includes administering a therapeutically effective amount of an iron chelator to the subject. In certain embodiments, the subject has breast cancer or lung cancer, e.g., non-small cell lung carcinoma. In certain embodiments, the subject was not known to have leptomeningeal metastasis prior to treatment.

The present disclosure provides methods for lengthening the period of survival of a subject having a cancer. In certain embodiments, the method includes administering a therapeutically effective amount of an iron chelator to the subject. In certain embodiments, the period of survival of the subject is lengthened by about 1 month, about 2 months, about 3 months, about 4 months, about 6 months, about 8 months, about 10 months, about 12 months, about 14 months, about 18 months, about 20 months, about 2 years, about 3 years, about 4 years, about 5 years or about 6 years or more. In certain embodiments, the subject has breast cancer or lung cancer, e.g., non-small cell lung carcinoma. In certain embodiments, the subject was not known to have leptomeningeal metastasis prior to treatment. In certain embodiments, the subject was known to have leptomeningeal metastasis prior to treatment. In certain embodiments, the method further includes diagnosing the subject with leptomeningeal metastasis.

In certain embodiments, the subject being treated with the iron chelator was previously treated with radiation therapy, e.g., cranial and/or spinal radiation therapy. In certain embodiments, the subject has progressive leptomeningeal metastasis, e.g., following radiation treatment. In certain embodiments, the subject has recurrent leptomeningeal metastasis, e.g., following radiation treatment.

In certain embodiments, administration of the iron chelator reduces the proliferation and/or survival of metastatic cancer cells in the cerebrospinal fluid of the subject.

In certain embodiments, the iron chelator is administered intrathecally in the methods of the present disclosure. In certain embodiments, the iron chelator is deferoxamine or a salt thereof. In certain embodiments, the iron chelator is deferoxamine mesylate. In certain embodiments, the iron chelator is administered to the subject at a dose from about 0.05 mg/kg to about 100 mg/kg.

In certain embodiments, methods of the present disclosure can further include administering a therapeutically effective amount of an anti-cancer agent to the subject.

The present disclosure further provides a kit for treating and/or preventing leptomeningeal metastasis in a subject that includes an iron chelator. In certain embodiments, the kit includes the iron chelator deferoxamine or a salt thereof. In certain embodiments, the iron chelator is deferoxamine mesylate. In certain embodiments, the kit includes an additional anti-cancer agent.

The present disclosure provides iron chelators for use in treating and/or preventing leptomeningeal metastasis in a subject, for use in lengthening the period of survival of a subject having a cancer and for use in lengthening the period of survival of a subject having leptomeningeal metastasis. In certain embodiments, the iron chelator is deferoxamine or a salt thereof. In certain embodiments, the iron chelator is deferoxamine mesylate.

### BRIEF DESCRIPTION OF THE DRAWINGS

FIG. 1A-1H. Sparse CSF iron promotes LCN2 expression in cancer cells. (A) Human leptomeningeal metastasis (LM) from breast cancer (Patient B). White plaques of LM visualized by magnetic resonance imaging (right-top panel), Giemsa-stained cytospin of CSF (right-bottom panel). Major cell populations are indicated. (B) Single-cell transcriptional map of cancer and immune cells present in CSF of five patients, projected with uniform manifold approximation and projection (UMAP). Each dot represents a cell, colored by PhenoGraph cluster; major cell types are manually annotated according to FIG. 2. Each individual patient is projected in FIGS. 3. (C and D) LCN2 and SLC22A17 gene expression in LM patients. Violin plots of LCN2 (C) and SLC22A17 (D) imputed gene expression in cells from individual patients. All cells grouped into 3 compartments—cancer cells (cancer patients A-E), lymphoid cells (CD4+ T cells, CD8+ T cells, B cells, NK cells) and myeloid cells (Monocyte 1, Monocyte 2, Macrophage, conventional dendritic cells (cDCs), plasmacytoid dendritic cells (pDCs)). (E and F) LCN2 (E) and SLC22A17 (F) Detection by immunofluorescent (IF) staining of leptomeninges collected at autopsy from patients harboring LM. Cytokeratin (white) indicates cancer cells. Macrophages are indicated by the macrophage-specific proteins CD68 or ionized calcium binding adaptor molecule 1 (IBA1) in green. LCN2 and SLC22A17 are shown in red. n=3 cancer patients. Scale bars, 50 μm. (G and H) LCN2 (G) and SLC22A17 (H) expression, assessed by cytospin staining of cancer cells and macrophages in CSF collected post-mortem from patients in E and F. Scale bars, 20 μm.

FIG. 2A-2B. Annotation of cell type by gene expression. (A) Canonical marker gene expression was employed to identify 14 cell clusters. (B) Correlation between PhenoGraph clusters and gene expression profiles from sorted bulk hematopoietic populations.

FIG. 3A-3E. Annotation of cell type by gene expression. Cells from each patient are indicated by letters corresponding to Table 1. Cell cluster identification is provided at upper right for comparison.

FIG. 4A-4O. Biological relevance of iron transporters in LM disease. (A) Cellular composition of CSF from patients harboring LM by clinical (CLIA-certified) flow cytometry. n=48 patients. (B) Cellular composition of CSF from patients harboring LM by 10× genomics single cell RNA-seq. n=5 patients. (C) Heatmap of iron transport gene expression. Cells are grouped by cluster and each column represents MAGIC-imputed gene expression of a single-cell, when rows represent specific iron transport genes. Expression values of each gene in a cell are scaled between 0 and 1. (D) Hepcidin levels as detected by ELISA in CSF from cancer patients with or without LM diagnosis. n=18. ** indicates p<0.01 (unpaired t test). Data represent mean±SEM (2 independent experiments). (E) Transferrin levels as detected by ELISA in CSF from cancer patients with or without LM diagnosis. n=28. NS=not significant (unpaired t test). Data represent mean±SEM (2 independent experiments). (F) LCN2 detection by ELISA in CSF from cancer patients with lung or breast primary tumors. Presence of LM is indicated +/−. n=18 lung cancer patients and 37 breast cancer patients. **** indicates p<0.0001 (unpaired t test). Data represent mean±SEM. (G) Evaluation of LCN2 expression by FACS in cancer cells and immune cells (neutrophils, monocytes, macrophages and T cells) from CSF of cancer patients. Presence of LM is indicated +/−. n=5 cancer patients without LM and n=5 cancer patients with LM. NS=not significant, * indicates p<0.05 (unpaired t test). Data represent mean±SEM. (H) Giemsa-stained cytospin of monocytes and macrophages sorted from CSF of patients harboring LM. Monocytes stain CD45+ CD11b+ CD14+ CD16low and macrophages stain CD45+ CD11b+ CD14+ CD16high. (I-N) Transferrin (I and L), Hepcidin (J and M) and Ferritin (K and N) expression in cancer cells and macrophages of leptomeningeal tumor (I-K) and CSF (L-N) from autopsy specimens harboring LM. Patients matched with FIG. 1F. Scale bars in cancer plague, 50 μm; Scale bars in CSF cytospin, 20 μm. (0) Density of iron transporters in cancer cells (cytokeratin+) and macrophages (IBA1+ or CD68+) respectively from cancer plaques and CSF cytospin. n=3 cancer patients with LM. NS=not significant, * indicates p<0.05 (unpaired t test). Data represent mean±SEM.

FIG. 5A-5G. LCN2 supports cancer cell growth in the leptomeninges. (A) Mouse model of LM. Left: In vivo bioluminescence imaging (BLI) of the LLC model at day 14 post intracardiac dissemination. Middle: Immunohistochemistry (IHC) for pan-cytokeratin. Scale Bar, 1000 μm in larger picture; 50 μm in inset. Right: t-distributed stochastic neighbor embedding (tSNE) of multicolor flow cytometry of CSF collected from syngeneic LLC mouse model (day 14), major cell types indicated as follows: cancer cell (CD45⁻); orange, neutrophil (CD45⁺CD11b⁺Ly6G⁺); purple, lymphocyte (CD45⁺CD3⁺); green, myeloid cell (CD45⁺CD11b⁺ excluding neutrophils); blue, and other leukocytes (CD45⁺CD11b⁻Ly6G⁻CD3⁻); red. n=5 mice. Representative sample is shown. (B) Tumor growth in mice injected with PC9-LeptoM cells expressing either shCtl or shLCN2. Two independent shRNAs (sh1 and sh2) were employed to target human LCN2. Left: Histogram represents in vivo BLI imaging post-inoculation at day 28. n=8-10 per group in each of two independent experiments. Right: Representative BLI images. *** indicates p<0.001 (unpaired t test). Data represent mean±SEM. (C) Kaplan-Meier survival curve of mice shown in (B). p<0.0001. (D) Tumor growth in mice injected with PC9-LeptoM cells expressing either shCtl or shSLC22A17. Two independent shRNAs (sh1 and sh2) were employed to target human SLC22A17. Left: Histogram represents in vivo BLI imaging at day 28 post-inoculation. n=8-10 per group in each of two independent experiments. Right: Representative BLI images. * indicates p<0.05, **** indicates p<0.0001 (Unpaired t test). Data represent mean±SEM. (E) Kaplan-Meier survival curve of mice shown in (D). p<0.0001. (F) Tumor growth in mice injected with PC9-Par cells expressing either green fluorescent protein (GFP) or LCN2. Left: Histogram represents in vivo BLI imaging post-inoculation at day 21. n=8-10 per group in each of two independent experiments. Right: Representative BLI images. * indicates p<0.05 (unpaired t test). Data represent mean±SEM. (G) Kaplan-Meier survival curve of mice shown in (F). p<0.0001.

FIG. 6A-6J. LCN2 and SLC22A17 expression in LM cancer cells. (A) LCN2 detection by ELISA in CSF from mice with or without LLC-LeptoM tumors at 2 weeks. n=8 mice per group in two independent experiments. **** indicates p<0.0001 (unpaired t test). Data represent mean±SEM. (B) Hepcidin levels as detected by ELISA in CSF from mice with or without LeptoM tumors at 2 weeks. n=8 mice per group. *** indicates p<0.001 (unpaired t test). Data represent mean±SEM (2 independent experiments). (C) LCN2 expression in PC9-LeptoM mouse models by IHC. Representative images from LeptoM model (top), Par model (middle) and vehicle-treated negative control (bottom) inoculated by 14 days. H&E (left panel) and Immunohistochemistry (IHC) for LCN2 (right panel). Scale Bars, 1000 μm in larger picture; 50 μm in inset. (D) mRNA expression of LCN2 in parental cells (Par) or LeptoM cells from PC9, MDA231 and LLC model systems. n=3 per group in each of two independent experiments. **** indicates p<0.0001 (unpaired t test). Data represent mean±SEM. (E) LCN2 expression by ELISA in conditioned media from parental cells (Par) and LeptoM cells from PC9, MDA231 and LLC model systems. n=3 per group in each of two independent experiments. ** indicates p<0.01, *** indicates p<0.001 (unpaired t test). Data represent mean±SEM. (F) Evaluation of LCN2 expression by FACS in cancer cells and immune cells (neutrophils, monocytes, macrophages and T cells) from CSF harboring LM of syngeneic LLC mouse model at day 10. n=6 mice per group in two independent experiments * indicates p<0.05, **** indicates p<0.0001 (unpaired t test). Data represent mean±SEM. (G and I) qPCR for SLC22A17, MC4R and LRP2 mRNA expression in parental cells and LeptoM cells from PC9 (G) and MDA231 model systems (I). n=3 per group in each of two independent experiments. Data represent mean±SEM. (H and J) IF for LCN2 and SLC22A17 expression in parental cells and LeptoM cells (PC9 in H and MDA231 in J). Scale Bar, 10 μm. n=3 per group in each of two independent experiments. Representative images are presented.

FIG. 7A-7L. LCN2 inhibition suppresses LM; LCN2 overexpression promotes LM. (A) LCN2 detection by ELISA in conditioned media from LeptoM cells with either shCtl or two shRNAs targeting LCN2 (sh1 and sh2). N=3 per group in each of two independent experiments. *** indicates p<0.001, **** indicates p<0.0001 (unpaired t test). Data represent mean±SEM. (B) LCN2 detection by ELISA in CSF collected at the endpoint from mice injected with PC9-LeptoM cells expressing either shCtl or shLCN2. N=7 per group. **** indicates p<0.0001 (unpaired t test). Data represent mean±SEM. (C and E) Tumor growth of mice injected with LeptoM cells expressing either shCtl or shLCN2. Left: Histogram represents in vivo BLI imaging post-inoculation (MDA231 at day 21, B; LLC at day 14, D). n=8-10 per group in each of two independent experiments. Right: Representative BLI images. * indicates p<0.05, *** indicates p<0.001 (unpaired t test). Data represent mean±SEM. (D and F) Kaplan-Meier survival curve of MDA231-LeptoM and LLC-LeptoM from (C) and (E). p<0.0001. (G) LCN2 detection by ELISA in CSF collected at the endpoint from mice injected with LLC-LeptoM cells expressing either shCtl or shLCN2. N=7 per group. **** indicates p<0.0001 (unpaired t test). Data represent mean±SEM. (H) Time course of tumor growth of mice transplanted in orthotopic lung with PC9-LeptoM cells expressing either shCtl or shLCN2 (sh1 and sh2). N=5-7 per group in each of two independent experiments. NS=not significant. Data represent mean±SEM. (I) Time course of tumor growth in mice injected subcutaneously with PC9-LeptoM cells expressing either shCtl or shLCN2. N=8-10 per group in each of two independent experiments. NS=not significant. Data represent mean±SEM. (J) Time course of tumor growth in mice injected in orthotopic mammary fat pad with MDA-LeptoM cells expressing either shCtl or shLCN2. N=8-10 per group in each of two independent experiments. NS=not significant. Data represent mean±SEM. (K) mRNA expression and western blot of lysate for LeptoM cells with either shCtl or two shRNAs targeting SLC22A17 (sh1 and sh2). N=3 per group in each of two independent experiments. **** indicates p<0.0001 (unpaired t test). Data represent mean±SEM. (L) SLC22A17 expression in LM in vivo. Representative images from PC9-LeptoM shCtl model (left) and PC9-LeptoM shSLC22A17 model (right), H&E staining (top) and MC for SLC22A17 (bottom). Scale Bar, 50 μm.

FIG. 8A-8H. Overexpression of LCN2 supports cancer cell growth in the CSF. (A) ELISA for LCN2 in Par cells expressing either GFP or LCN2. *** indicates p<0.001, **** indicates p<0.0001 (unpaired t test). Data represent mean±SEM. (B) LCN2 detection by ELISA in CSF collected at the endpoint from mice injected with PC9-Par cells expressing either GFP or LCN2. n=8 per group. ** indicates p<0.01, **** indicates p<0.0001 (unpaired t test). Data represent mean±SEM. (C) Tumor growth of mice injected with MDA231-Par cells expressing either GFP or LCN2. Left: Histogram represents in vivo BLI imaging day 14 post-inoculation. n=8-10 per group in each of two independent experiments. Right: Representative BLI images. * indicates p<0.05 (unpaired t test). Data represent mean±SEM. (D) Kaplan-Meier survival curve of MDA231-Par from (C). p<0.0001. (E) LCN2 detection by ELISA in CSF collected at the endpoint from mice injected with MDA231-Par cells expressing either GFP or LCN2. n=8 per group. ** indicates p<0.01, **** indicates p<0.0001 (unpaired t test). Data represent mean±SEM. (F) Tumor growth of mice injected with LLC-Par cells expressing either GFP or LCN2. Left: Histogram represents in vivo BLI imaging 21 days post-inoculation. n=8-10 per group in each of two independent experiments. Right: Representative BLI images. * indicates p<0.05 (unpaired t test). Data represent mean±SEM. (G) Kaplan-Meier survival curve of MDA231-Par and LLC-Par from (I) and (L). p<0.0001. (H) LCN2 detection by ELISA in CSF collected at the endpoint from mice injected with LLC-Par cells expressing either GFP or LCN2. n=8 per group. ** indicates p<0.01, **** indicates p<0.0001 (unpaired t test). Data represent mean±SEM.

FIG. 9A-9F. Cancer cells generate LCN2 in response to inflammatory cytokines. (A) Mean expression of canonical pro-inflammatory cytokines from single-cell transcriptomics. Markov affinity-based graph imputation of cells (MAGIC)-imputed cytokine expression was standardized to zero mean and unit of standard deviation. Please also refer to pro-inflammatory cytokine dataset provided in Table S5. (B) Schematic for cancer cell co-culture with supernatant from macrophages in LLC-LeptoM model. Conditioned media was collected from macrophages, freshly sorted from CSF or spleen of C57Bl/6 mice harboring LLC-LeptoM cells or no cancer by fluorescence-activated cell sorting (FACS) and added to cultures of Par cells or LeptoM cells for 14 days, exchanging fresh conditioned media every 3 days. (C) Evaluation of LCN2 levels by enzyme-linked immunosorbent assay (ELISA) in conditioned media generated as per (B). n=3 in each of two independent experiments. NS=not significant, ** indicates p<0.01, **** indicates p<0.0001 (unpaired t test). Data represent mean±SEM. (D) LCN2 mRNA detection by qPCR in CSF-derived macrophages and LLC-LeptoM cells after co-culture at day 14. n=3 in each of two independent experiments. NS=not significant, *** indicates p<0.001 (unpaired t test). Data represent mean±SEM. (E and F) CSF from patients harboring LM was treated with neutralizing antibodies against IL-6, IL-8, or IFNγ and added to PC9-LeptoM cells for 12 hours. LCN2 was quantified by ELISA in conditioned media 24 hours after removing CSF. n=3 in each of two independent experiments. NS=not significant, *** indicates p<0.001, **** indicates p<0.0001 (unpaired t test). Data represent mean±SEM.

FIG. 10A-10G. Pro-inflammatory cytokines induce cancer cell LCN2 expression in LM. (A). JAK-STAT signaling and NF-κB signaling sets of transcriptomic signatures from single-cell sequencing in CSF of patients. JAK-STAT and NF-κB signaling gene datasets were obtained from MsigDB and are provided in Table 5. (B) IL-6, IL-8, and IL-1β levels by ELISA in CSF from cancer patients with primary tumors of LM positive or negative. n=42. ** indicates p<0.01, *** indicates p<0.001, **** indicates p<0.0001 (unpaired t test). Data represent mean±SEM. (C) Representative images of leptomeninges from LeptoM (top), parental (middle) and vehicle (bottom) in syngeneic mouse model stained by HE and IHC for LCN2, IL-6 and IL-1(3. Scale Bar, 100 μm. (D) ELISA-based quantification of IL-1β, IL-6 and TNFα in CSF from mice with or without LLC-LeptoM tumor at 2 weeks. n=7-10 mice per group. * indicates p<0.05, **indicates p<0.01 (unpaired t test). Data represent mean±SEM. (E) FACS-based strategy for macrophage isolation from mouse spleen or CSF. A total of 1.8×10⁵ cells were collected from mouse CSF; 35.3% cells were obtained and isolated for single-cell populations from scatter height and scatter width parameters; 5×10³ macrophages were labeled by CD45+ CD11b+F4/80+ and prepared for further analysis. (F) LCN2 expression of immune cells of spleen and blood from vehicle, LPS and LLC-LeptoM mouse models at day 10. n=5 mice per group. NS=not significant, * indicates p<0.0332 (multiple t test). Data represent mean±SEM. (G) Immune cell composition in CSF of vehicle, LPS and LLC-LeptoM shCtl and shLCN2 mouse models at day 10. n=5 mice per group. NS=not significant, * indicates p<0.0332 (multiple t test). Data represent mean±SEM.

FIG. 11A-11E. Proinflammatory cytokines in human CSF. (A) LCN2 was quantified by ELISA in the CSF from patients harboring LM was treated with neutralizing antibody against IL-6 or IL-8 and added to MDA231-LeptoM cells for 12 hours. N=3 in each of two independent experiments. NS=not significant, * indicates p<0.05, ** indicates p<0.01, *** indicates p<0.001 (unpaired t test). Data represent mean±SEM. (B) Evaluation of LCN2 expression by ELISA with the treatment of pro-inflammatory cytokines IL-6, IL-8, IL-1β and TNF in PC9-Par and PC9-LeptoM cells. LCN2 expression was determined by ELISA of conditioned media collected 48 hours after recombinant cytokine stimulation. N=3 in each of two independent experiments. *indicates p<0.05 (unpaired t test). Data represent mean±SEM. (C) LCN2 expression by ELISA 48 h after treatment of MDA-Par and MDA-LeptoM cells with IL-6, IL-8, IL-1β and TNF. LCN2 expression by ELISA in conditioned media for 48 hours after recombinant cytokines stimulation. N=3 in each of two independent experiments. *indicates p<0.05 (unpaired t test). Data represent mean±SEM. (D) LCN2 expression by ELISA 48 h after treatment of LLC-Par and LLC-LeptoM cells with IL-6, IL-1β and TNF. N=3 in each of two independent experiments. * indicates p<0.05 (unpaired t test). Data represent mean±SEM. (E) Visualization of macrophage cells in first two diffusion components. First diffusion component is best matched by Hypoxia as indicated with GSEA es=0.6, fdr=0 and M1 phenotype es=−0.5, fdr=0.11, while second diffusion component is best matched by M2 phenotype es=0.7, fdr=0. MAGIC imputed gene set expression was standardized to zero mean and unit of standard deviation.

FIG. 12A-12E. LCN2 supports cancer cell growth in the hypoxic CSF microenvironment. (A and B) Correlation between hypoxia and iron ion transport gene signatures. Axis value represents mean gene signature expression per cell, standardized to have zero mean and unit of standard deviation. Each dot represents a cell, colored by cell type; please also refer to FIG. 1B. (B) Cancer cell populations from (A) alone. (C) Gene set enrichment (GSA) analysis of PC9 and LLC models by bulk RNA sequencing of LeptoM shCtl and shLCN2. Number of significantly upregulated (red) and downregulated (blue) genes in GSA analysis are indicated on the x axis. n=2 per group. p<0.05. (D) Leptomeningeal tumor growth in a dual reporter in vivo system. PC9-LeptoM cells express Firefly luciferase (Fluc) constitutively; NanoLuc (Nluc) is induced downstream of hypoxia response element (HRE). Left: Fluc and Nluc are assayed by BLI at indicated time points. Right: representative BLI images at day 0 and day 7. n=8-10 per group in each of two independent experiments. Data represent mean±SEM. (E) Leptomeningeal tissue sections stained with hematoxylin and eosin (H&E) and IHC for LCN2, hypoxia-inducible factors (HIF) HIF-1α and HIF-2α in PC9-LeptoM model. Scale bar, 50 μM.

FIG. 13A-13R. LCN2 induces cancer cell proliferation in hypoxia. (A) Hypoxia transcriptomic signature from single-cell sequencing in CSF of patients. Genes included in hypoxia signature are provided in Table 5. (B) Venn diagram of genes differentially expressed between LeptoM-shCtl and LeptoM-shLCN2 cell lines in PC9 and MDA model systems by bulk RNA sequencing. <0.5 fold change or <20 normalized counts cutoff p<0.05, two technical replicates and three biological replicates. (C) Hypoxia associated GO terms in cells as described in (B). Gene set enrichment analysis by DAVID pathway analysis. (D) Tumor growth (Fluc) and hypoxia (Nluc) in LLC-LeptoM model. Left: Dual reporter in vivo system. Cancer cells constitutively express Fluc; and Nluc is induced downstream of HRE. Fluc and Nluc are assayed by BLI at indicated time points. Right: representative BLI images at day 0 and day 7. n=8-10 per group in each of two independent experiments. Data represent mean±SEM. (E) H&E and IHC for LCN2, HIF-1α and HIF-2α in syngeneic LLC model. n=8-10 per group in each of two independent experiments. Representative images are shown. (F-I) Hypoxic stress (Nluc, F and H) and LeptoM cell growth (Fluc, G and I) at 0.5%, 2% and 20% oxygen in vitro. PC9-LeptoM in (F) and (G); LLC-LeptoM in (H) and (I). Data represent mean±SEM. (J-O) Cell viability by cell titer glo (J, L and N) and apoptosis by caspase-3/7 activity (K, M and 0) in LeptoM cells after knockdown of LCN2 in 0.5% 02. PC9-LeptoM in (J) and (K); MDA231-LeptoM in (L) and (M); LLC-LeptoM in (N) and (0). n=4 per group in each of two independent experiments. **indicates p<0.01, *** indicates p<0.001, **** indicates p<0.0001 (unpaired t test). Data represent mean±SEM. (P) HIF-1α and HIF-2a levels in PC9-LeptoM cells in shCtl and two shLCN2 (1 and 2) by western-blot after being cultured at 0.5% 02 for 48 hours. (Q and R) Viability (Q) and apoptosis (R) of Par cells with after overexpression of LCN2 in 0.5% 02. n=4 per group in each of two independent experiments. ** indicates p<0.0021, *** indicates p<0.0002, **** indicates p<0.0001 (unpaired t test). Data represent mean±SEM.

FIG. 14A-14K. Leptomeningeal metastasis requires iron transporters. (A) Total iron measured by mass spectroscopy in CSF cancer patients with and without LM. Presence of LM is indicated +/−. N=11 cancer patients without LM and n=12 cancer patients with LM. * indicates p<0.05 (unpaired t test). Data represent mean±SEM. (B) LCN2 iron binding assay in patient CSF. Non-LCN2-bound iron [NLBI] and LCN2-bound iron [LBI] was detected in the CSF from cancer patients with or without LM. N=10. **** indicates p<0.0001 (unpaired t test). Data represent mean±SEM. (C) Expression of genes associated with iron transport in parental cells and LeptoM cells by bulk RNA sequencing. N=2 in PC9 cells and n=3 in LeptoM cells. Presented as log 2 fold change in each group with cutoff <20 normalized counts. (D) Tumor growth in mice injected with PC9-LeptoM cells expressing shCtl or shLCN2. Treatment with either vehicle or 5 mg/Ml holo-transferrin (diferric [Tf]) was commenced on day 1 and continued every third day for a total of 7 doses. Left: Histogram represents in vivo BLI imaging post-inoculation at day 21. N=6-8 per group in each of two independent experiments. Right: Representative BLI images. Sh1+Veh vs sh1+Tf, p=0.2087. sh2+Veh vs sh2+Tf, p=0.0604. Data represent mean±SEM. (E) Kaplan-Meier survival curve of LeptoM groups from (D). sh1+Veh vs sh1+Tf, p<0.0001. sh2+Veh vs sh2+Tf, p<0.0001. (F) Top panel: Mrna expression for Transferrin in LeptoM cells with either shCtl or two shRNAs targeting LCN2. N=3 per group in each of two independent experiments. NS=not significant. Data represent mean±SEM. Bottom panel: Transferrin detection by western blot for in LeptoM cells with either shCtl or two shRNAs targeting LCN2. (G and H) Cell associated iron of PC9-LeptoM cells after knockdown of SLC22A17 or LCN2. In select experiments, 100 ng/Ml recombinant [LCN2] or 100 μg/Ml diferric [Tf] was added. N=3 per group in each of two independent experiments. Iron measurements by mass spectroscopy in (G), colorimetric assay in (H) ** indicates p<0.01, *** indicates p<0.001, **** indicates p<0.0001 (unpaired t test). Data represent mean±SEM. (I) cell viability of PC9-LeptoM cells after knockdown of SLC22A17 or LCN2. In select experiments, 100 ng/ml recombinant [LCN2] or 100 μg/ml diferric [Tf] was added. N=3 per group in each of two independent experiments. * indicates p<0.05 ** indicates p<0.01, *** indicates p<0.001, **** indicates p<0.0001 (unpaired t test). Data represent mean±SEM. (J) Cell associated iron from macrophages collected from CSF or spleen in LLC-LeptoM mouse model or LPS-stimulated model at day 14, please refer to FIG. 7D. n=4 per group. NS=not significant, **** indicates p<0.0001 (unpaired t test). Data represent mean±SEM. Iron measured by colorimetric assay. (K) Competition for iron between LeptoM cells and macrophages (dose-dependent). Cells were treated with iron-containing conditioned media (0, 0.05, 0.5, 5 or 50 Mm) for 24 hours after overnight incubation with serum free media. Intracellular iron was determined by colorimetric assay. N=4 per group in each of two independent experiments. Data represent mean±SEM in two independent experiments. LLC-LeptpM vs Monocyte-derived Macrophages, p<0.0001. LLC-LeptpM vs CSF-derived Macrophages, p=0.002297. LLC-LeptoM shLCN2+Veh vs LLC-LeptoM shLCN2+Rlcn2, p=0.000056.

FIG. 15A-15G. LCN2 transports iron to support cancer cell growth. (A) Tumor growth in mice injected with MDA231-LeptoM cells expressing shCtl or shLCN2. Treatment with either vehicle or 5 mg/mL holo-transferrin (diferric [Tf]) began on day 1 and continued every third day for a total of 7 doses. Left: Histogram represents in vivo BLI imaging post-inoculation at day 21. n=8-10 per group in each of two independent experiments. Right: Representative BLI images. ** indicates p<0.01 (unpaired t test). Data represent mean±SEM. (B) Kaplan-Meier survival curve of LeptoM groups from (A). sh1+Veh vs sh1+Tf, p<0.0001. sh2+Veh vs sh2+Tf, p<0.0001. (C) Soluble iron concentration from macrophages collected from CSF in lipopolysaccharide (LPS)-stimulated, LeptoM or LeptoM-shLCN2 (sh1 and sh2) mouse model at day 14. Soluble iron was measured by mass spectroscopy. n=4 per group. ** indicates p<0.01, *** indicates p<0.001, **** indicates p<0.0001 (unpaired t test). Data represent mean±SEM. (D and E) Phagocytosis (D) and reactive oxygen species (ROS) generation (E) in CSF-derived macrophages by flow cytometric analysis after LeptoM (LLC model), shLCN2 LPS, or vehicle (Ctl) treatment. Phagocytosis detected by FITC-labeled E. coli. CellROX Green⁺ cells represent percent of live cells with ROS. n=12-15 (Ctl), n=4-5 (LPS), n=11-18 (LeptoM), n=8 (shLCN2). NS=not significant, * indicates p<0.05, ** indicates p<0.01, **** indicates p<0.0001 (unpaired t test). Data represent mean±SEM in two independent experiments. (F) Tumor growth in MDA231-LeptoM model after chelator treatment: deferoxamine (DFO), D-penicillamine (D-Pen) on day 0 (early) or day 7 (late); see also FIG. 16. Left: in vivo BLI imaging in day 28 post-inoculation. n=8-10 per group in each of two independent experiments. Right: Representative BLI images. **** indicates p<0.0001. Data represent mean±SEM. (G) Kaplan-Meier survival curve of mice treated in (F). p<0.0001.

FIG. 16A-16L. Iron chelator treatment reduces CSF iron. (A) Experimental scheme. Mice were inoculated with 2×10³ cells intrathecally on day 0. Treatment with either vehicle, DFO, or D-pen was commenced on either day 1 or day 7 and continued every third day for a total of 7 doses. (B) Chelators delivered intrathecally. DFO, Deferoxamine. D-Pen, D-Penicillamine. (C-F) Intracellular iron (C and E) and viability (D and F) of PC9-LeptoM cells and MDA231-LeptoM cells after treatment with chelators in different concentrations (0 μM, 1 μM, 10 μM and 100 μM). n=3-4 per group in each of two independent experiments. * indicates p<0.05, ** indicates p<0.01, ***indicates p<0.001, **** indicates p<0.0001 (unpaired t test). Data represent mean±SEM. (G) Growth of PC9-LeptoM tumor after treatment indicated in (A). Left: in vivo BLI imaging in day 28 post-inoculation. n=8-10 per group in each of two independent experiments. Right: Representative BLI images. **** indicates p<0.0001. Data represent mean±SEM. (H) Kaplan-Meier survival curve from (C) and (E). p<0.0001. (I and J) Total iron concentration measured in CSF samples of mice with treatment of chelators in PC9-LeptoM Model (I) in MDA231-LeptoM model (J). NS=not significant, * indicates p<0.05, ** indicates p<0.01 (unpaired t test). Data represent mean±SEM. (K and L) Immune cell composition in CSF of LLC-LeptoM model with the treatment of vehicle, DFO, or D-pen at day 10. n=6 per group. NS=not significant, * indicates p<0.05, ** indicates p<0.01 (unpaired t test). Data represent mean±SEM.

FIG. 17A-17L. Changes in peripheral blood counts (A-F) and organ function (G-L) with IT-DFO dose escalation ranging from 0 μM (PBS control) and 100 μM. Group means and 95% confidence intervals displayed. P-values <0.05 calculated using one-way ANOVA comparing each IT-DFO group to control. Abbreviations: RBC, red blood cell; WBC, white blood cell; ANC, absolute neutrophic count; ALC, absolute lymphocyte count; ALT, alanine aminotransferase; AST, aspartate aminotransferase; ALP, alkaline phosphatase; Tbili, total bilirubin, BUN, blood urea nitrogen.

### DETAILED DESCRIPTION

The present disclosure relates to composition and methods useful in connection with the use of iron chelation to prevent and/or treat leptomeningeal metastasis. Leptomeningeal metastasis is a rare complication of cancer in which the disease spreads to the membranes (i.e., meninges) surrounding the brain and spinal cord. The present disclosure is based, in part, on the discovery that cancer cells within the cerebrospinal fluid rely on iron metabolism for growth and survival, and that the administration of an iron chelator can reduce iron concentration in the cerebrospinal fluid, impair the growth of leptomeningeal metastases, and significantly prolong survival.

For purposes of clarity of disclosure and not by way of limitation, the detailed description is divided into the following subsections:
I. Definitions;
II. Iron Chelators;
III. Methods of Treatment;
IV. Kits; and
V. Exemplary Embodiments.

### I. DEFINITIONS

The terms used in this disclosure generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “proliferation” refers to an increase in cell number.

An “effective amount” or “therapeutically effective amount” is an amount effective, at dosages and for periods of time necessary, that produces a desired effect, e.g., the desired therapeutic or prophylactic result. In certain embodiments, an effective amount can be formulated and/or administered in a single dose. In certain embodiments, an effective amount can be formulated and/or administered in a plurality of doses, for example, as part of a dosing regimen.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

The terms “detection” or “detecting” include any means of detecting, including direct and indirect detection.

As used herein, the term “derivative” refers to a chemical compound with a similar core structure.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

The terms “primary” or “primary origin,” as used herein in relation to cancer, refers to the organ in the body of the subject where the cancer began (e.g., the breast or lung). The primary origin of a cancer can be identified using methods known in the art, e.g., medical imaging, examination of biopsy samples with immunohistochemistry techniques, and/or gene expression profiling.

### II. IRON CHELATORS

The present disclosure provides iron chelators for use in the methods disclosed herein. An iron chelator can be a molecule, e.g., chemical compound, that reacts, e.g., binds, with iron ions to form a complex, e.g., a stable, water-soluble complex. In certain embodiments, an iron chelator for use in the present disclosure is a molecule, e.g., chemical compound, that can react with iron ions in the cerebrospinal fluid of a subject.

Non-limiting examples of iron chelators for use in the present disclosure include deferoxamine (also referred to as desferoxamine and desferrioxamine), deferasirox, desferrithiocin, deferiprone, clioquinol, O-trensox, tachpyr, dexrazoxane, triapine, pyridoxal, di-2-pyridylketone, pyridoxal isonicotinoyl hydrazine or salts or derivatives thereof. In certain embodiments, the iron chelator can be a phytochemical including, but not limited to, flavan-3-ol, curcumin, apocynin, kolaviron, floranol, baicalein, baicalin, quercetin, epigallocatechin gallate, theaflavin, phytic acid, genistein, maltol, tropolone, mimosine or salts or derivatives thereof. In certain embodiments, the salt of an iron chelator for use in the present disclosure can be an acetate salt, a mesylate salt, a phosphate salt or a formate salt. In certain embodiments, the salt of an iron chelator is a mesylate salt.

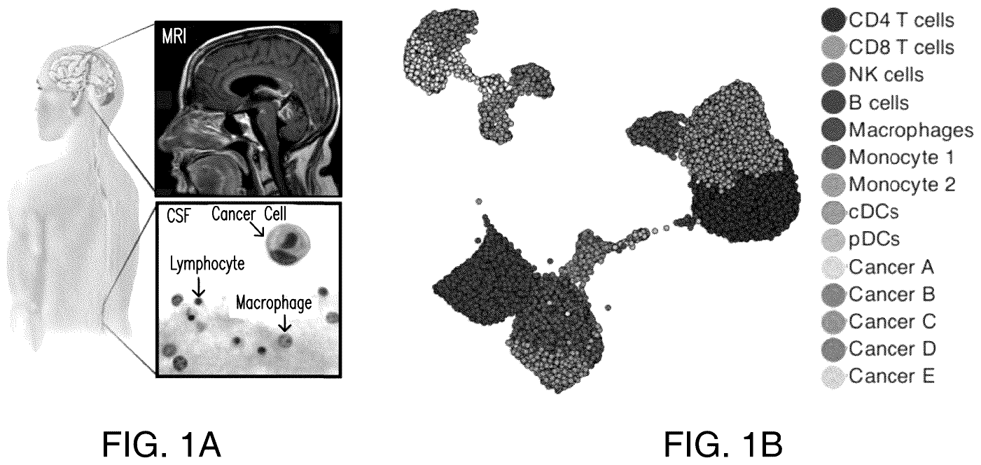

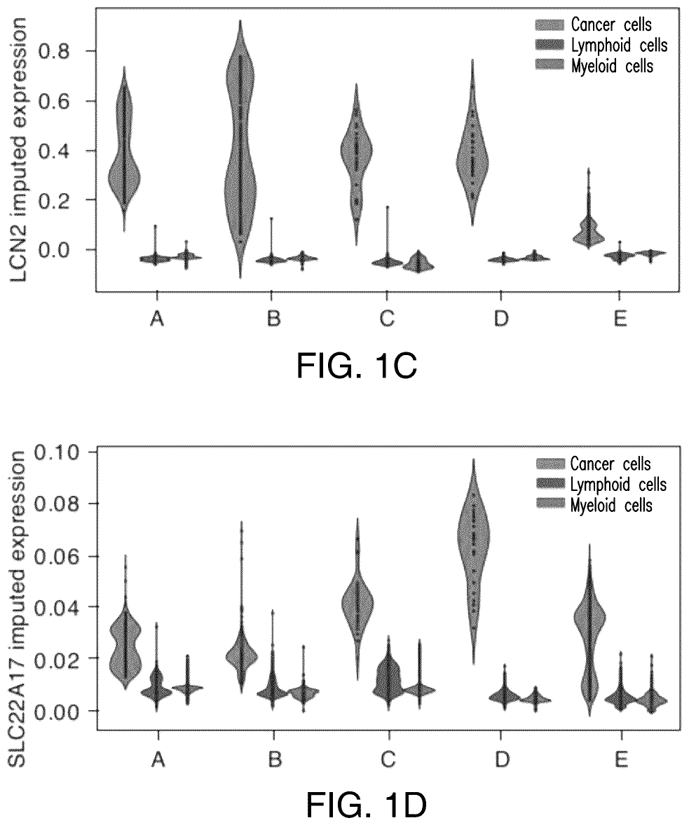

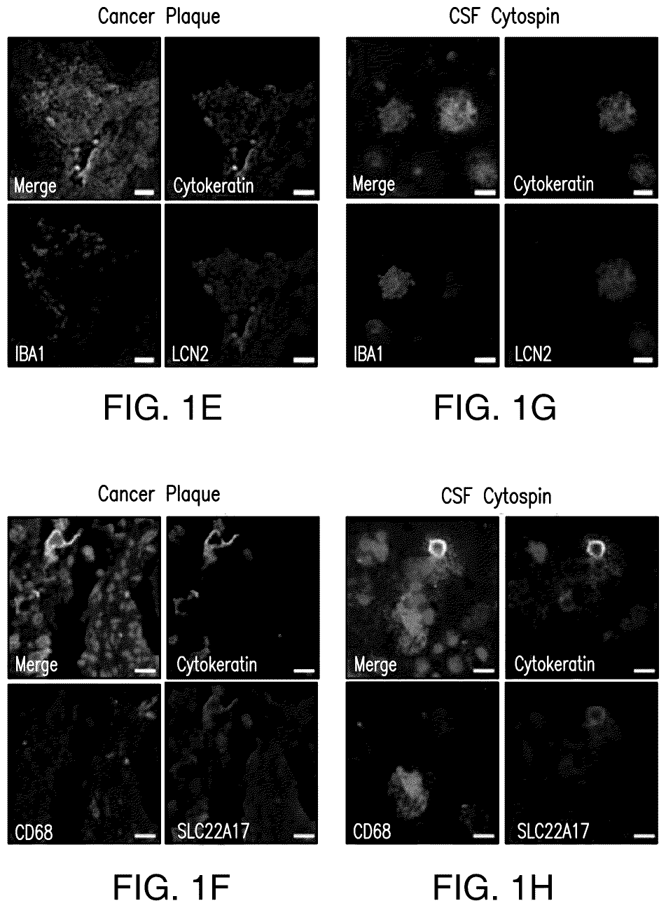

Additional non-limiting examples of iron chelators are provided in Mobarra et al., Int. J. Hematol. Oncol. Stem Cell. Res. 10(4):239-247 (2016), e.g., Table 1 of Mobarra et al.; Kontoghiorghe et al., Molecule 20:20841-20872 (2015), e.g., Table 2 and Figures 1 and 3-11 of Kontoghiorghe et al.; and Hatcher et al., Future Med. Chem. 1(9):1643-70 (2009), e.g., Table 1 of Hatcher et al., the contents of which are incorporated by reference in their entireties herein.

In certain embodiments, the iron chelator for use in the present disclosure is deferoxamine or salts or derivatives thereof, e.g., deferoxamine mesylate. In certain embodiments, the iron chelator for use in the present disclosure is deferasirox or salts or derivatives thereof. In certain embodiments, the iron chelator for use in the present disclosure is desferrithiocin or salts or derivatives thereof. In certain embodiments, the iron chelator for use in the present disclosure is deferiprone or salts or derivatives thereof.

In certain non-limiting embodiments, the present disclosure further provides pharmaceutical formulations of iron chelators for therapeutic use. In certain embodiments, the pharmaceutical formulation includes an iron chelator and a pharmaceutically acceptable carrier. “Pharmaceutically acceptable,” as used herein, includes any carrier which does not interfere with the effectiveness of the biological activity of the active ingredients, e.g., iron chelator, and that is not toxic to the patient to whom it is administered. Non-limiting examples of suitable pharmaceutical carriers include phosphate-buffered saline solutions, water, emulsions, such as oil/water emulsions, various types of wetting agents and sterile solutions. Additional non-limiting examples of pharmaceutically acceptable carriers can include gels, bioabsorbable matrix materials, implantation elements containing the inhibitor and/or any other suitable vehicle, delivery or dispensing means or material. Such carriers can be formulated by conventional methods and can be administered to the subject.

In certain embodiments, the pharmaceutical formulations of the present disclosure can be formulated using pharmaceutically acceptable carriers well known in the art that are suitable for parenteral administration, e.g., intravenous administration, intraarterial administration, intrathecal administration, intranasal administration, intramuscular administration, subcutaneous administration and intracisternal administration. In certain embodiments, the pharmaceutical formulation is formulated for intrathecal administration. For example, but not by way of limitation, the pharmaceutical formulation can be formulated as a solution, suspension or emulsion.

In certain non-limiting embodiments, the pharmaceutical formulations of the present disclosure can be formulated using pharmaceutically acceptable carriers well known in the art that are suitable for oral administration. Such carriers enable the pharmaceutical compositions to be formulated as tablets, pills, capsules, liquids, gels, syrups, slurries, suspensions and the like, for oral ingestion by a patient to be treated. In certain embodiments, the pharmaceutical formulation can be a solid dosage form.

In certain embodiments, the pharmaceutical formulation can be formulated to release the iron chelator immediately upon administration. Alternatively, the pharmaceutical formulation can be formulated to release the iron chelator at any predetermined time or time period after administration. Such types of compositions are generally known as controlled release formulations, which include (i) formulations that create substantially constant concentrations of the iron chelator within the subject over an extended period of time; (ii) formulations that after a predetermined lag time create substantially constant concentrations of the iron chelator within the subject over an extended period of time; (iii) formulations that sustain the iron chelator's action during a predetermined time period by maintaining a relatively constant, effective level of the iron chelator in the body with concomitant minimization of undesirable side effects; (iv) formulations that localize action of iron chelator, e.g., spatial placement of a controlled release composition adjacent to or in the disease, e.g., cancer cells; (v) formulations that achieve convenience of dosing, e.g., administering the composition once per week or once every two weeks; and (vi) formulations that target the action of the iron chelator by using carriers or chemical derivatives to deliver the iron chelator to a particular target cell type. In certain embodiments, controlled release is obtained by appropriate selection of various formulation parameters and ingredients, including, e.g., various types of controlled release compositions and coatings. For example, but not by way of limitation, the iron chelator can be formulated with appropriate excipients into a pharmaceutical formulation that, upon administration, releases the iron chelator in a controlled manner, e.g., oil solutions, suspensions, emulsions, microcapsules, molecular complexes, microspheres, nanoparticles, patches and liposomes.

In certain embodiments, the pharmaceutical formulations suitable for use in the present disclosure can include formulations where the iron chelators are contained in a therapeutically effective amount. A “therapeutically effective amount” refers to an amount that is able to achieve one or more of an anti-cancer effect, prolongation of survival and/or prolongation of period until relapse. The therapeutically effective amount of an active ingredient can vary depending on the active ingredient, e.g., iron chelator, formulation used, the cancer and its severity, and the age, weight, etc., of the subject to be treated. In certain embodiments, a patient can receive a therapeutically effective amount of an iron chelator as a single dose or multiple administrations of two or more doses, which can depend on the dosage and frequency as required and tolerated by the patient.

### III. METHODS OF TREATMENT

The present disclosure relates to methods for preventing and/or treating leptomeningeal metastasis in a subject. The present disclosure provides methods for preventing and/or treating leptomeningeal metastasis in a subject by reducing the amount of iron in the cerebrospinal fluid of the subject. As described in detail in the Example section below, the studies presented in the instant application indicate that the chelation of iron in the cerebrospinal fluid can be used to treat leptomeningeal metastasis by reducing the amount of iron available to cancer cells within the cerebrospinal fluid. In particular, reducing the iron concentration in the cerebrospinal fluid can impair the growth of leptomeningeal metastases and significantly prolong survival.

In certain non-limiting embodiments, the present disclosure provides for a method of preventing and/or treating leptomeningeal metastasis in a subject. For example, but not by way of limitation, the present disclosure provides a method for preventing and/or treating leptomeningeal metastasis in a subject that has cancer. In certain embodiments, the method can include administering a therapeutically effective amount of an iron chelator to the subject. In certain embodiments, administration of the iron chelator inhibits the proliferation and/or survival of metastatic cancer cells in the cerebrospinal fluid of a subject. In certain embodiments, the subject was known to have leptomeningeal metastasis prior to treatment. In certain non-limiting embodiments, the subject was not known to have leptomeningeal metastasis prior to treatment.

In certain embodiments, the present disclosure provides methods for reducing the risk of a subject that has cancer from developing leptomeningeal metastasis, which can include administering a therapeutically effective amount of an iron chelator to the subject. In certain embodiments, the cancer has metastasized to locations other than the meninges, e.g., the cerebrospinal fluid-filled leptomeninges. For example, but not by way of limitation, the cancer can have metastasized to the brain of the subject. In certain embodiments, the cancer has not responded to other anti-cancer agents, as described herein.

In certain non-limiting embodiments, the present disclosure provides a method of treating a subject having a cancer that includes diagnosing leptomeningeal metastasis in the subject and then treating the subject with an iron chelator. For example, but not by way of limitation, a method of treating a subject having cancer can include (a) diagnosing the subject with leptomeningeal metastasis and (b) administering a therapeutically effective amount of an iron chelator to the subject. In certain embodiments, the method for diagnosing leptomeningeal metastasis includes performing magnetic resonance imaging (MRI) of the brain and/or spine, without or with contrast, e.g., gadolinium contrast, cytology of the cerebrospinal fluid or a circulating tumor cell (CTC) count >3.0/3.0 mL. Additional methods for diagnosing leptomeningeal metastasis are disclosed in Nayar et al. Oncotarget. 8(42):73312-73328 (2017), the contents of which are incorporated by reference herein.

In certain non-limiting embodiments, the present disclosure provides for a method of preventing metastasis of a cancer, e.g., primary cancer, to the meninges, e.g., the cerebrospinal fluid-filled leptomeninges, in a subject. In certain embodiments, the method includes administering a therapeutically effective amount of an iron chelator to the subject. In certain embodiments, preventing metastasis to the meninges includes inhibiting and/or preventing the proliferation and/or survival of metastatic cancer cells in the cerebrospinal fluid of a subject.

In certain embodiments, the present disclosure provides a method for lengthening the period of survival of a subject having a cancer. In certain embodiments, the method includes administering a therapeutically effective amount of an iron chelator to the subject. In certain embodiments, the survival period of a subject having cancer can be lengthened by about 1 month, about 2 months, about 3 months, about 4 months, about 6 months, about 8 months, about 10 months, about 12 months, about 14 months, about 18 months, about 20 months, about 2 years, about 3 years, about 4 years, about 5 years or about 6 years or more.

Methods disclosed herein can be used for treating any suitable cancers, e.g., primary cancers, e.g., that have spread, can spread or have a risk of spreading to the meninges surrounding the brain and spinal cord. Non-limiting examples of such cancers include liver cancers, brain cancers, cervical cancers, colorectal cancers, breast cancers, endometrial carcinomas, gastric cancers, cancers of the head and neck, bladder cancers, lung cancers, ovarian cancers, biliary tree cancers, hepatocellular carcinomas, leukemia, lymphomas, myelomas and sarcomas. In certain embodiments, methods disclosed herein can be used for treating a cancer selected from bladder urothelial carcinoma, cervical squamous cell carcinoma and endocervical adenocarcinoma, cholangiocarcinoma, colon adenocarcinoma, head and neck squamous cell carcinoma, kidney chromophobe, kidney renal papillary cell carcinoma, liver hepatocellular carcinoma, lung adenocarcinoma, lung squamous cell carcinoma, prostate adenocarcinoma, rectum adenocarcinoma, stomach adenocarcinoma and uterine corpus endometrial carcinoma. In certain embodiments, methods disclosed herein can be used for treating a lung cancer. In certain embodiments, the lung cancer is selected from small cell lung carcinoma, non-small cell lung carcinoma, lung adenocarcinoma, lung squamous cell carcinoma and large-cell lung carcinoma. In certain embodiments, the cancer is an adenocarcinoma. In certain embodiments, the cancer is a solid tumor. In certain embodiments, the cancer is a breast cancer, e.g., an infiltrating ductal carcinoma and/or a lobular carcinoma. In certain embodiments, the cancer is a melanoma, e.g., a primary leptomeningeal melanoma and/or a melanocytoma. In certain embodiments, the cancer is a brain cancer, e.g., an astrocytoma, a glioblastoma, an ependymoma, a choroid plexus papilloma and/or a choroid plexus carcinoma. In certain embodiments, the cancer is a lymphoma, e.g., a B-cell lymphoma and/or a T-cell lymphoma. In certain embodiments, the cancer is a B-cell lymphoma, e.g., a primary central nervous system (CNS) lymphoma and/or a secondary CNS lymphoma. In certain embodiments, the cancer is a T-cell lymphoma. In certain embodiments, the cancer is a leukemia, e.g., acute lymphoblastic leukemia (ALL) and/or acute myeloid leukemia (AML).

In certain embodiments, the cancer is lung cancer, e.g., non-small cell lung carcinoma. For example, but not by way of limitation, the present disclosure provides a method for preventing and/or treating leptomeningeal metastasis in a subject that has lung cancer, e.g., non-small cell lung carcinoma. In certain embodiments, the present disclosure provides a method for preventing and/or treating leptomeningeal metastasis in a subject that has EGFR-mutant non-small cell lung carcinoma.

In certain embodiments, the cancer is breast cancer. For example, but not by way of limitation, the present disclosure provides a method for preventing and/or treating leptomeningeal metastasis in a subject that has breast cancer.

Non-limiting examples of iron chelators for use in the present disclosure are described in Section II. For example, but not by way of limitation, the iron chelator can be selected from the group consisting of deferoxamine, deferasirox, desferrithiocin, deferiprone, clioquinol, O-trensox, tachpyr, dexrazoxane, triapine, pyridoxal, di-2-pyridylketone, pyridoxal isonicotinoyl hydrazine, flavan-3-ol, curcumin, apocynin, kolaviron, floranol, baicalein, baicalin, quercetin, epigallocatechin gallate, theaflavin, phytic acid, genistein, maltol, tropolone, mimosine or salts or derivatives thereof and a combination thereof. In certain embodiments, the iron chelator is deferoxamine or salts or derivatives thereof, e.g., deferoxamine mesylate. In certain embodiments, the iron chelator is deferasirox or salts or derivatives thereof. In certain embodiments, the iron chelator is desferrithiocin or salts or derivatives thereof. In certain embodiments, the iron chelator is deferiprone or salts or derivatives thereof.

In certain embodiments, an iron chelator can be administered to a subject at a dose of about 0.05 mg/kg to about 1,000 mg/kg. For example, but not by way of limitation, an iron chelator can be administered to a subject at a dose of about 0.05 mg/kg to about 900 mg/kg, about 0.05 mg/kg to about 800 mg/kg, about 0.05 mg/kg to about 700 mg/kg, about 0.05 mg/kg to about 600 mg/kg, about 0.05 mg/kg to about 500 mg/kg, about 0.05 mg/kg to about 400 mg/kg, about 0.05 mg/kg to about 300 mg/kg, about 0.05 mg/kg to about 200 mg/kg, about 0.05 mg/kg to about 100 mg/kg, about 1 mg/kg to about 1,000 mg/kg, about 10 mg/kg to about 1,000 mg/kg, about 50 mg/kg to about 1,000 mg/kg, about 100 mg/kg to about 1,000 mg/kg, about 200 mg/kg to about 1,000 mg/kg, about 300 mg/kg to about 1,000 mg/kg, about 400 mg/kg to about 1,000 mg/kg, about 500 mg/kg to about 1,000 mg/kg, about 600 mg/kg to about 1,000 mg/kg, about 700 mg/kg to about 1,000 mg/kg, about 800 mg/kg to about 1,000 mg/kg, about 900 mg/kg to about 1,000 mg/kg, about 10 mg/kg to about 900 mg/kg, about 50 mg/kg to about 800 mg/kg, about 100 mg/kg to about 700 mg/kg, about 200 mg/kg to about 600 mg/kg or about 300 mg/kg to about 500 mg/kg.

In certain embodiments, an iron chelator can be administered to a subject at a dose of about 0.05 mg/kg to about 100 mg/kg. For example, but not by way of limitation, the iron chelator can be administered to a subject at a dose of about 0.05 mg/kg to about 90 mg/kg, about 0.05 mg/kg to about 80 mg/kg, about 0.05 mg/kg to about 70 mg/kg, about 0.05 mg/kg to about 60 mg/kg, about 0.05 mg/kg to about 50 mg/kg, about 0.05 mg/kg to about 40 mg/kg, about 0.05 mg/kg to about 30 mg/kg, about 0.05 mg/kg to about 20 mg/kg, about 0.05 mg/kg to about 10 mg/kg, about 0.05 mg/kg to about 5 mg/kg, about 0.05 mg/kg to about 1 mg/kg, about 5 mg/kg to about 100 mg/kg, about 10 mg/kg to about 100 mg/kg, about 20 mg/kg to about 100 mg/kg, about 30 mg/kg to about 100 mg/kg, about 40 mg/kg to about 100 mg/kg, about 50 mg/kg to about 100 mg/kg, about 60 mg/kg to about 100 mg/kg, about 70 mg/kg to about 100 mg/kg, about 80 mg/kg to about 100 mg/kg, about 90 mg/kg to about 100 mg/kg, about 10 mg/kg to about 90 mg/kg, about 20 mg/kg to about 80 mg/kg, about 30 mg/kg to about 70 mg/kg or about 40 mg/kg to about 60 mg/kg.

In certain embodiments, a subject can be administered up to about 100 grams of the iron chelator in a single dose or as a total daily dose. For example, but not way of limitation, a subject can be administered up to about 90 grams, up to about 80 grams, up to about 70 grams, up to about 60 grams, up to about 50 grams, up to about 40 grams, up to about 30 grams, up to about 20 grams, up to about 10 grams, up to about 9 grams, up to about 8 grams, up to about 7 grams, up to about 6 grams, up to about 5 grams, up to about 4 grams, up to about 3 grams, up to about 2 grams, up to about 1 gram, up to about 900 mg, up to about 800 mg, up to about 700 mg, up to about 600 mg, up to about 500 mg, up to about 400 mg, up to about 300 mg, up to about 200 mg, up to about 100 mg, up to about 50 mg or up to about 25 mg of the iron chelator in a single dose or as a total daily dose.

In certain embodiments, a subject can be administered up to about 2,000 mg of the iron chelator in a single dose or as a total daily dose. For example, but not by way of limitation, a subject can be administered up to about 1,950 mg, up to about 1,900 mg, up to about 1,850 mg, up to about 1,800 mg, up to about 1,750 mg, up to about 1,700 mg, up to about 1,650 mg, up to about 1,600 mg, up to about 1,550 mg, up to about 1,500 mg, up to about 1,450 mg, up to about 1,400 mg, up to about 1,350 mg, up to about 1,300 mg, up to about 1,250 mg, up to about 1,200 mg, up to about 1,150 mg, up to about 1,100 mg, up to about 1,050 mg, up to about 1,000 mg, up to about 950 mg, up to about 900 mg, up to about 850 mg, up to about 800 mg, up to about 750 mg, up to about 700 mg, up to about 650 mg, up to about 600 mg, up to about 550 mg, up to about 500 mg, up to about 450 mg, up to about 400 mg, up to about 350 mg, up to about 300 mg, up to about 250 mg, up to about 200 mg, up to about 150 mg, up to about 100 mg, up to about 50 mg or up to about 25 mg of the iron chelator in a single dose or as a total daily dose.

In certain embodiments, the subject can be administered from about 50 mg to about 10,000 mg of the iron chelator in a single dose or as a total daily dose, e.g., from about 50 mg to about 10,000 mg, about 50 mg to about 9,000 mg, about 50 mg to about 8,000 mg, about 50 mg to about 7,000 mg, about 50 mg to about 6,000 mg, about 50 mg to about 5,000 mg, about 50 mg to about 4,000 mg, about 50 mg to about 3,000 mg, about 50 mg to about 2,000 mg, about 50 mg to about 1,000 mg, about 100 mg to about 10,000 mg, about 200 mg to about 10,000 mg, about 300 mg to about 10,000 mg, about 400 mg to about 10,000 mg, about 500 mg to about 10,000 mg, about 600 mg to about 10,000 mg, about 700 mg to about 10,000 mg, about 800 mg to about 10,000 mg, about 900 mg to about 10,000 mg, about 1,000 mg to about 10,000 mg, about 2,000 mg to about 10,000 mg, about 3,000 mg to about 10,000 mg, about 4,000 mg to about 10,000 mg, about 5,000 mg to about 10,000 mg, about 6,000 mg to about 10,000 mg, about 7,000 mg to about 10,000 mg, about 8,000 mg to about 10,000 mg, about 9,000 mg to about 10,000 mg, about 100 mg to about 5,000 mg, about 200 mg to about 4,000 mg, about 300 mg to about 3,000 mg, about 400 mg to about 2,000 mg, about 500 mg to about 1,000 mg, about 600 mg to about 900 mg or about 700 mg to about 800 mg of the iron chelator in a single dose or as a total daily dose. In certain embodiments, the subject can be administered from about 50 mg to about 5,000 mg of the iron chelator, e.g., deferoxamine, in a single dose or as a total daily dose. In certain embodiments, the subject can be administered from about 50 mg to about 4,000 mg of the iron chelator, e.g., deferoxamine, in a single dose or as a total daily dose. In certain embodiments, the subject can be administered from about 50 mg to about 3,000 mg of the iron chelator, e.g., deferoxamine, in a single dose or as a total daily dose. In certain embodiments, the subject can be administered from about 50 mg to about 2,000 mg of the iron chelator, e.g., deferoxamine, in a single dose or as a total daily dose. In certain embodiments, the subject can be administered from about 50 to about 1,000 mg of the iron chelator, e.g., deferoxamine, in a single dose or as a total daily dose.

In certain embodiments, a subject can be administered about 5,000 mg of the iron chelator, e.g., deferoxamine, in a single dose or as a total daily dose. In certain embodiments, a subject can be administered about 4,000 mg of the iron chelator, e.g., deferoxamine, in a single dose or as a total daily dose. In certain embodiments, a subject can be administered about 3,000 mg of the iron chelator, e.g., deferoxamine, in a single dose or as a total daily dose. In certain embodiments, a subject can be administered about 2,000 mg of the iron chelator, e.g., deferoxamine, in a single dose or as a total daily dose. In certain embodiments, a subject can be administered about 1,000 mg of the iron chelator, e.g., deferoxamine, in a single dose or as a total daily dose. In certain embodiments, a subject can be administered about 25 mg or more of the iron chelator, e.g., deferoxamine, in a single dose or as a total daily dose.

It is to be understood that, for any particular subject, specific dosage regimes should be adjusted over time according to the individual need and the professional judgment of the person administering or supervising the administration of the iron chelator. For example, the dosage the iron chelator can be increased if the lower dose does not provide sufficient activity in the treatment of a disease or condition described herein (e.g., cancer and/or leptomeningeal metastasis). Alternatively, the dosage of the composition can be decreased if the disease (e.g., cancer and/or leptomeningeal metastasis) is reduced, no longer detectable or eliminated.

In certain embodiments, the iron chelator can be administered once a day, twice a day, three times a day, once a week, twice a week, three times a week, four times a week, five times a week, six times a week, once every two weeks, once a month, twice a month, once every other month or once every third month. In certain embodiments, the iron chelator can be administered three times a week. In certain embodiments, the iron chelator can be administered once a week. In certain embodiments, the iron chelator can be administered twice a week. In certain embodiments, the iron chelator can be administered two times a week for about four weeks and then administered once a week for the remaining duration of the treatment. In certain embodiments, a subject can be administered up to about 5,000 mg of the iron chelator in a single dose or as a total daily dose two times a week. In certain embodiments, a subject can be administered up to about 4,000 mg of the iron chelator in a single dose or as a total daily dose two times a week. In certain embodiments, a subject can be administered up to about 3,000 mg of the iron chelator in a single dose or as a total daily dose two times a week. In certain embodiments, a subject can be administered up to about 2,000 mg of the iron chelator in a single dose or as a total daily dose two times a week. In certain embodiments, a subject can be administered up to about 1,000 mg of the iron chelator in a single dose or as a total daily dose two times a week. In certain embodiments, a subject can be administered up to about 500 mg of the iron chelator in a single dose or as a total daily dose two times a week.

In certain embodiments, the period of treatment can be at least one day, at least one week, at least one month, at least two months, at least three months, at least four months, at least five months or at least six months. In certain embodiments, the iron chelator can be administered until the leptomeningeal metastasis is no longer detectable. In certain embodiments, the iron chelator can be administered until cancer cells are no longer detectable in the cerebrospinal fluid.

In certain embodiments, the iron chelator can be administered to a subject by any route known in the art. In certain embodiments, the iron chelator can be administered parenterally. In certain embodiments, the iron chelator can be administered orally, intravenously, intraarterially, intrathecally, intranasally, subcutaneously, intramuscularly and rectally. In certain embodiments, the iron chelator can be administered intrathecally. For example, but not by way of limitation, the present disclosure provides methods for the prevention and/or treatment of leptomeningeal metastasis in a subject, e.g., having cancer, by intrathecal administration of an iron chelator.

In certain embodiments, the iron chelator can be used alone or in combination with one or more anti-cancer agents. For example, but not by way of limitation, methods of the present disclosure can include administering one or more iron chelators and one or more anti-cancer agents. “In combination with,” as used herein, means that the iron chelator and the one or more anti-cancer agents are administered to a subject as part of a treatment regimen or plan. In certain embodiments, being used in combination does not require that the iron chelator and one or more anti-cancer agents are physically combined prior to administration, administered by the same route or that they be administered over the same time frame. In certain embodiments, the anti-cancer agent is administered before an iron chelator. In certain embodiments, the anti-cancer agent is administered after an iron chelator. In certain embodiments, the anti-cancer agent is administered simultaneously with an iron chelator.

An “anti-cancer agent,” as used herein, can be any molecule, compound, chemical or composition that has an anti-cancer effect. Anti-cancer agents include, but are not limited to, chemotherapeutic agents, radiotherapeutic agents, cytokines, anti-angiogenic agents, apoptosis-inducing agents, anti-cancer antibodies, anti-cyclin-dependent kinase agents, and/or agents which promote the activity of the immune system including but not limited to cytokines such as but not limited to interleukin 2, interferon, an anti-CTLA4 antibody, an anti-PD-1 antibody and/or an anti-PD-L1 antibody. In certain embodiments, the anti-cancer agent can be a taxane, a platinum-based agent, an anthracycline, an anthraquinone, an alkylating agent, a HER2 targeting therapy, vinorelbine, a nucleoside analog, ixabepilone, eribulin, cytarabine, radiation therapy, a hormonal therapy, methotrexate, capecitabine, lapatinib, 5-FU, vincristine, etoposide or any combination thereof. In certain embodiments, the anti-cancer agent can be radiation therapy.

In certain embodiments, administration of the iron chelator to the subject has an anti-cancer effect or therapeutic benefit. An “anti-cancer effect” or “therapeutic benefit” as used herein, refers to one or more of a reduction in aggregate cancer cell mass, a reduction in cancer cell growth rate, a reduction in cancer cell proliferation, a reduction in tumor mass, a reduction in tumor volume, a reduction in tumor cell proliferation, a reduction in tumor growth rate, a reduction in the number of cancer cells in the cerebrospinal fluid and/or a reduction in tumor metastasis. In certain embodiments, an anti-cancer effect can refer to a complete response, a partial response, a stable disease (without progression or relapse) and/or a response with a later relapse or progression-free survival in a patient diagnosed with cancer. In certain embodiments, an anti-cancer effect can refer to the prevention and/or reduction of leptomeningeal metastasis within a subject, e.g., the prevention and/or reduction of metastasis of a cancer to the meninges surrounding the brain and spinal cord of a subject.

In certain embodiments, the subject being administered the iron chelator previously received another anti-cancer therapy, e.g., radiation therapy and/or administration of an anti-cancer agent. For example, but not by way of limitation, the anti-cancer agent can be a chemotherapeutic agent such as a taxane, a platinum-based agent, an anthracycline, an anthraquinone, an alkylating agent, a HER2 targeting therapy (e.g., an anti-HER2 antibody), vinorelbine, a nucleoside analog, ixabepilone, eribulin, cytarabine, a hormonal therapy, methotrexate, capecitabine, lapatinib, 5-FU, vincristine, etoposide, an anti-angiogenic therapy, immunotherapy or any combination thereof. In certain embodiments, the subject previously received radiation therapy, e.g., cranial and/or spinal radiation, e.g., proton or photon.

In certain embodiments, the subject has progressive or recurrent leptomeningeal metastases after anti-cancer therapy before administration of an iron chelator. In certain embodiments, the subject has progressive leptomeningeal metastases after radiation therapy. In certain embodiments, the subject has recurrent leptomeningeal metastases after radiation therapy. In certain embodiments, the anti-cancer therapy, e.g., radiation therapy, is completed at least 60 days prior to administration of an iron chelator. In certain embodiments, recurrence of leptomeningeal metastases can be determined by radiographic progression on contrast-enhanced MRI, the development of positive CSF cytology following leptomeningeal metastasis-directed cancer treatment or a rise in CSF CTC count by at least 20%.

In certain embodiments, the present disclosure provides a method for treating leptomeningeal metastasis or lengthening the period of survival of a subject having a cancer that includes (a) treating the subject with an anti-cancer therapy and (b) administering a therapeutically effective amount of an iron chelator to the subject if the subject has progressive or recurrent leptomeningeal metastases after treatment with the anti-cancer therapy. In certain embodiments, the anti-cancer therapy is radiation therapy. In certain embodiments, the anti-cancer therapy is cranial and/or spinal radiation. In certain embodiments, the anti-cancer therapy, e.g., radiation, is completed at least 60 days prior to administration of the iron chelator.

### IV. KITS

The present disclosure provides kits for use in the disclosed methods. In certain embodiments, a kit can include a container that includes an iron chelator or a pharmaceutical formulation thereof. In certain embodiments, the container can include a single dose of the iron chelator or multiple doses of the iron chelator. A container can be any receptacle and closure suitable for storing, shipping, dispensing and/or handling a pharmaceutical product.

In certain embodiments, the kit can further include a second container that includes a solvent, carrier and/or solution for diluting and/or resuspending the iron chelator. For example, but not by way of limitation, the second container can include sterile water.

In certain embodiments, the kit can further include instructions for administering the iron chelator. For example, but not by way of limitation, the instructions can describe the method for administration and the dosage amount. In certain embodiments, the instructions indicate that the iron chelator or pharmaceutical formulation thereof can be administered intrathecally. In certain embodiments, the instructions can indicate that the iron chelator or a pharmaceutical formulation thereof can be administered to a subject at a dose of between about 0.05 mg/kg to about 100 mg/kg, e.g., about 5 mg/kg to about 100 mg/kg.

In certain embodiments, the kit can further include a device for administering the iron chelator or a pharmaceutical formulation thereof. In certain embodiments, the kit can include a device for administering the iron chelator or a pharmaceutical formulation thereof intrathecally. For example, but not by way of limitation, the device can include a syringe, catheter, e.g., implantable catheter, and/or pump.

### V. EXEMPLARY EMBODIMENTS

A. In certain embodiments, the presently disclosed subject matter provides a method for treating leptomeningeal metastasis in a subject, comprising administering a therapeutically effective amount of an iron chelator to the subject.

A1. The method of A, wherein the iron chelator is administered intrathecally.

A2. The method of A or A1, wherein the iron chelator is deferoxamine or a salt thereof.

A3. The method of any one of A-A2, wherein the iron chelator is deferoxamine mesylate.

A4. The method of any one of A-A3, wherein the subject has a cancer.

A5. The method of A4, wherein the cancer is breast cancer.

A6. The method of A4, wherein the cancer is lung cancer.

A7. The method of A6, wherein the lung cancer is non-small cell lung carcinoma.

A8. The method of any one of A-A7, further comprising administering a therapeutically effective amount of an anti-cancer agent to the subject.

A9. The method of any one of A-A8, wherein the subject was previously treated with radiation therapy.

A10. The method of A9, wherein the radiation therapy is cranial and/or spinal radiation therapy.

A11. The method of A-A10, wherein the subject has progressive leptomeningeal metastasis.

A12. The method of any one of A-A11, wherein the subject has recurrent leptomeningeal metastasis.

A13. The method of any one of A-A12, wherein the subject has progressive and/or recurrent leptomeningeal metastasis following radiation therapy.

A14. The method of any one of A-A13, wherein the iron chelator is administered to the subject at a dose from about 0.05 mg/kg to about 1,000 mg/kg.

A15. The method of any one of A-A14, wherein the iron chelator is administered to the subject at a dose from about 0.05 mg/kg to about 100 mg/kg.

A16. The method of any one of A-A15, wherein administration of the iron chelator reduces the proliferation and/or survival of metastatic cancer cells in the cerebrospinal fluid of the subject.

A17. The method of any one of A-A16, further comprising diagnosing the subject with leptomeningeal metastasis.

B. In certain embodiments, the presently disclosed subject matter provides a method for preventing or reducing the risk of leptomeningeal metastasis in a subject having cancer, comprising administering a therapeutically effective amount of an iron chelator to the subject.

B1. The method of B, wherein the iron chelator is administered intrathecally.

B2. The method of B or B1, wherein the iron chelator is deferoxamine or a salt thereof.

B3. The method of any one of B-B2, wherein the iron chelator is deferoxamine mesylate.

B4. The method of any one of B-B3, wherein the cancer is selected from breast cancer and lung cancer.

B5. The method of B4, wherein the lung cancer is non-small cell lung carcinoma.

B6. The method of any one of B-B5, further comprising administering a therapeutically effective amount of an anti-cancer agent to the subject.

B7. The method of any one of B-B6, wherein the subject was not known to have leptomeningeal metastasis prior to treatment with the iron chelator.

B8. The method of any one of B-B7, wherein the iron chelator is administered to the subject at a dose from about 0.05 mg/kg to about 1,000 mg/kg.

B9. The method of any one of B-B8, wherein the iron chelator is administered to the subject at a dose from about 0.05 mg/kg to about 100 mg/kg.

C. In certain embodiments, the presently disclosed subject matter provides a method for lengthening the period of survival of a subject having a cancer, comprising administering a therapeutically effective amount of an iron chelator to the subject.

C1. The method of C, wherein the iron chelator is administered intrathecally.

C2. The method of C or C1, wherein the iron chelator is deferoxamine or a salt thereof.

C3. The method of any one of C-C2, wherein the iron chelator is deferoxamine mesylate.

C4. The method of any one of C-C3, wherein the cancer is selected from breast cancer and lung cancer.

C5. The method of C4, wherein the lung cancer is non-small cell lung carcinoma.

C6. The method of any one of C-C5, wherein the subject was not known to have leptomeningeal metastasis prior to treatment.

C7. The method of any one of C-C5, wherein the subject was known to have leptomeningeal metastasis prior to treatment.

C8. The method of any one of C-C7, further comprising administering a therapeutically effective amount of an anti-cancer agent to the subject.

C9. The method of any one of C-C8, wherein the subject was previously treated with radiation therapy.

C10. The method of C9, wherein the radiation therapy is cranial and/or spinal radiation therapy.

C11. The method of any one of C-C10, wherein the subject has progressive leptomeningeal metastasis.

C12. The method of any one of C-C10, wherein the subject has recurrent leptomeningeal metastasis.

C13. The method of any one of C-C12, wherein the subject has progressive and/or recurrent leptomeningeal metastasis following radiation therapy.

C14. The method of any one of C-C13, wherein the iron chelator is administered to the subject at a dose from about 0.05 mg/kg to about 1,000 mg/kg.

C15. The method of any one of C-C14, wherein the iron chelator is administered to the subject at a dose from about 0.05 mg/kg to about 100 mg/kg.

C16. The method of any one of C-C15, wherein administration of the iron chelator reduces the proliferation and/or survival of metastatic cancer cells in the cerebrospinal fluid of the subject.

C17. The method of any one of C-C16, further comprising diagnosing the subject with leptomeningeal metastasis.

C18. The method of any one of C-C17, wherein the period of survival of the subject is lengthened by about 1 month, about 2 months, about 3 months, about 4 months, about 6 months, about 8 months, about 10 months, about 12 months, about 14 months, about 18 months, about 20 months, about 2 years, about 3 years, about 4 years, about 5 years or about 6 years or more.

D. In certain embodiments, the presently disclosed subject matter provides a kit for treating and/or preventing leptomeningeal metastasis in a subject, comprising an iron chelator.

D1. The kit of D, wherein the iron chelator is deferoxamine or a salt thereof.

D2. The kit of D or D1, wherein the iron chelator is deferoxamine mesylate.

D3. The kit of any one of D-D2, further comprising an anti-cancer agent.

E. In certain embodiments, the presently disclosed subject matter provides an iron chelator for use in treating and/or preventing leptomeningeal metastasis in a subject.

E1. The iron chelator for use of E, wherein the iron chelator is deferoxamine or a salt thereof.

E1. The iron chelator for use of E or E1, wherein the iron chelator is deferoxamine mesylate.

F. In certain embodiments, the presently disclosed subject matter provides an iron chelator for use in lengthening the period of survival of a subject having a cancer.

F1. The iron chelator for use of F, wherein the iron chelator is deferoxamine or a salt thereof.

F2. The iron chelator for use of F or F1, wherein the iron chelator is deferoxamine mesylate.

G. In certain embodiments, the presently disclosed subject matter provides an iron chelator for use in lengthening the period of survival of a subject having leptomeningeal metastasis.

G1. The iron chelator for use of G, wherein the iron chelator is deferoxamine or a salt thereof.

G2. The iron chelator for use of G or G1, wherein the iron chelator is deferoxamine mesylate.

### EXAMPLES

The presently disclosed subject matter will be better understood by reference to the following Examples, which are provided as exemplary of the presently disclosed subject matter, and not by way of limitation.

### Example 1: Cancer Cells Deploy Lipocalin-2 to Collect Limiting Iron in Leptomeningeal Metastasis

Materials and Methods:

Human Studies. CSF in excess of that needed for clinical care was collected from breast and lung cancer patients undergoing lumbar puncture, cisternal, or Ommaya tap. Human tissues and CSF were obtained under MSKCC Institutional Review Board-approved protocol 18-505 “Gene expression patterns in Leptomeningeal Metastasis.” Clinical information including tumor tissue diagnosis, coulter counter CSF counts, time to LM diagnosis, etc. was abstracted from the medical record and de-identified. All patients provided informed consent.

Animal Studies. All animal studies were approved by the MSKCC Institutional Animal Care and Use Committee under protocol 18-01-002. Nude outbred (Stock #: 069, ENVIGO) and C57BL/6 (Stock #: 0006664, Jackson Laboratory) mice were housed in the MSKCC vivarium, with individually ventilated cages, sterilized food and water. All mice were used at 4-6 weeks of age. Human breast and lung cancer models were hosted in female nude outbred mice, and murine lung cancer models were hosted in female and male mice at 1:1 ratios.

Cell Lines. The generation of metastatic derivative cell lines from PC9, MDA231, and LLC cells to produce LeptoM cell lines has been described previously (1). These cancer cell lines were cultured in DMEM with 10% fetal bovine serum (FBS), 1% Penicillin-Streptomycin, and 1% L-glutamine, and incubated at 37° C. sterile incubator with 5% CO₂. For generating lentivirus, 293T cells were cultured in DMEM with 10% FBS, 1% Penicillin-Streptomycin, 1% L-glutamine. Stable cancer cell lines expressing lentiviral vector were maintained in media with 2 μg/ml puromycin for at least 7 days prior to use in assays. HRE-NLuc expressed cells were constructed by electrotransfection of plasmid pNL3.2-HRE-NLuc and cultured in media with 500 ug/ml Hygromycin. All studies employed cells before they reached eighth passage. All cells tested negative for presence of Mycoplasma (MycoAlert, Lonza, LT07).

Plasmids. For pCDH-CMV-LCN2 vector, Homo sapiens LCN2 cDNA (CCDS6892.1) or Mus musculus Lcn2 cDNA (CCDS15913.1) oligos (to amplify from PC9 and LLC mRNA respectively) were inserted into pCDH-CMV-MCS-EF1-Puro lentiviral backbone vector (CD510B-1) using Quick Ligation Kit (New England Biolabs, M2200) with enzymes XbaI and BamHI (New England Biolabs, R0145, R3136). Lentiviral vectors expressing shRNA directed against LCN2 (RHS4533-EG3934 and RMM4534-EG16819) and SLC22A17 (RHS4533-EG51310) (Dharmacon) were expressed in pLKO.1 backbone vector. For pNL3.2-HRE-Nluc vector, HRE cDNA (GenScript) was cloned into pNL3.2-Nluc (Promega Corporation, #N1041).

shRNA.
Homo LCN2-1:
(SEQ ID NO: 1)
TAGAGGGTGATCTTGAAGTAC;
Homo LCN2-2:
(SEQ ID NO: 2)
AAGAACACCATAGCATGCTGG;
Mus lcn2-1:
(SEQ ID NO: 3)
AAGTTCTGAGTTGAGTCCTGG;
Mus lcn2-2:
(SEQ ID NO: 4)
TATTTCCCAGAGTGAACTGGC;
Homo SLC22A17-1:
(SEQ ID NO: 5)
AAGTGAATGCAAAGCAGCAGG;
Homo SLC22A17-2:
(SEQ ID NO: 6)
AATCTGCCGCTTCACTATCAG.
Hypoxia response element (HRE) cDNA.
(SEQ ID NO: 7)
GTGACTACGTGCTGCCTAGGTGACTACGTGCTGCCTAGGTGACTACGTG
CTGCCTAGGTGACTACGTGCTGCCTAG.

Antibodies. Mouse-specific antibodies used in flow cytometry analysis included antibodies against CD45 (BUV395, BD, 564279, 1:100), CD11 b (APC, BioLegend, 101212; PE/Cy7, BioLegend, 101216), F4/80 (BV605, BioLegend, 123133), CD3 (PerCP/Cy5.5, BioLegend, 100218; AF594, BioLegend, 100240), CD14 (APC, BioLegend, 123312), Ly6C (APC/Fire750, BioLegend, 128046), Ly6G (BV785, BioLegend, 127645), and LCN2 (unconjugated, R&D, AF1757). The LCN2 R-PE conjugate was prepared in-house with R-Phycoerythrin conjugation kit (Abcam, ab102918), as recommended.

Human-specific antibodies included antibodies against CD45 (BUV395, BD, 563792), CD3 (BUV661, BD, 565065), CD16 (BUV737, BD, 741904), CD4 (BV510, BioLegend, 317444), CD56 (BV650, BioLegend, 318344), CD279 (BV711, BioLegend, 367427), CD15 (BV785, BioLegend, 323044), CD8 (PerCP, BioLegend, 344708), Pan-Cytokeratin (FITC, Miltenyi Biotec, 130-080-101), LCN2 (R-PE conjugate, prepared as described above), CD14 (PE/Dazzle594, BioLegend, 367134), CD11b (PE/CyS, BioLegend, 101210), CD68 (PE/Cy7, BioLegend, 333816), CD25 (APC, BioLegend, 302610), CD64 (AF700, BD, 561188), and CD69 (APC/Cy7, BioLegend, 310914).

Antibodies for IHC and IF included LCN2 (R&D, AF1757), SLC22A17 (Sigma-Aldrich, SAB3500306), Transferrin (R&D, AF3987), Ferritin (R&D, MAB93541-100), Hepcidin (LSBio, LS-B4534-50), Iba-1 (Wako, 019-19741), Cytokeratin (R&D, AF7619), CD68 (R&D, MAB2040-SP), HIF-1α (R&D, MAB1536), HIF-2a (Abcam, ab199), IL-6 (R&D, AF-406), IL-1β (R&D, AF-401), ImmPress™ HRE Reagent Kits (Vector Laboratories, MP-7405, MP-7401, MP-7402), Cy3-AffiniPure Donkey Anti-Goat (Jackson ImmunoResearch, 705-165-147), Cy3-AffiniPure Donkey Anti-Rabbit IgG (Jackson ImmunoResearch, 711-165-152), Cy3-AffiniPure Donkey Anti-Mouse IgG (Jackson ImmunoResearch, 715-165-150), Donkey Anti-Sheep IgG Antibody (Alexa Fluor® 647) (Jackson ImmunoResearch, 713-605-147), Alexa Fluor® 488-AffiniPure Donkey Anti-Mouse IgG (Jackson ImmunoResearch, 715-545-151), Alexa Fluor® 488-AffiniPure Donkey Anti-Rabbit (Jackson ImmunoResearch, 711-545-152, Cy3. Antibodies for western blot analysis included HIF-1α (R&D, MAB1536), HIF-2α (Abcam, ab199), β-actin (Sigma-Aldrich, A5441); secondary antibodies were anti-mouse IgG (Sigma-Aldtich, A9917) and Anti-Rabbit IgG (Sigma-Aldrich, A0545).

Human CSF Single-Cell Transcriptomic Analysis. Single-cell and bulk RNA-sequencing data have been deposited to NCBI GEO as GSE150681 SuperSeries. CSF, collected with informed consent from patients under protocol IRB 13-039, was processed to isolate the sample into cell-free CSF and the cellular contents of the CSF. The whole CSF sample was centrifuged at 600×g for 5 minutes without brake at 4° C. to pellet the cells, and the supernatant was saved as cell-free CSF. The pellet was resuspended, washed with PBS supplemented with 0.4% BSA twice and processed immediately. The cells were manually counted with a hematocytometer. scRNA-Seq was performed with 10× genomics system using Chromium Single Cell 3′ Library and Gel Bead Kit V2 (catalog no. 120234). Briefly, 8,700 cells (viability 70-80%) were processed per sample, targeting recovery of ˜5,000 cells with 3.9% multiplet rate. In cases, where cell count was too low to target 5,000 cells, maximum volume (34 μl) was loaded in the microfluidic droplet generation device. After reverse transcription reaction emulsions were broken, barcoded cDNA was purified with DynaBeads, followed by 12-cycles of PCR amplification. The resulting amplified barcoded-cDNA library was fragmented to ˜400-600 bp, ligated to sequencing adapter and PCR-amplified to obtain sufficient amount of material for next-generation sequencing. The final libraries were sequenced on an Illumina NovaSeq 6000 system (Read 1—28 cycles, Index Read—8 cycles, and Read 2—96 cycles).

Data processing. Raw FASTQ files for each patient were preprocessed using the SEQC pipeline (2) using hg38 human genome and the default SEQC parameters for 10× Genomics to obtain the molecule count matrix. The SEQC pipeline aligns the reads to the genome, corrects barcode and unique molecular identifier (UMI) errors, resolves multi-mapping reads, and generates a molecule count matrix. SEQC also performs a number of filtering steps: 1. Identification of true cells from cumulative distribution of molecule counts per barcode, 2. removal of apoptotic cells identified at cells with >20% of molecules derived from the mitochondria, and 3. removal of low-complexity cells identified as cells where the detected molecules are aligned to a small subset of genes. In addition, cells with less than 800-1,000 molecules detected were filtered out. After the filtering, ˜19,000 cells were retained with a median molecule count of ˜4,100 and median gene count of ˜1,200, indicating the high quality of the data. Each patient contributed from 1,800 to 5,000 single-cells to this dataset.

Cell doublet detection. Cell doublets are a characteristic error source in droplet-based single-cell sequencing data here two cells are randomly co-encapsulated with the same barcode. To remove likely doublet cells unsupervised machine learning classifier was employed (3). This classifier operates on a count matrix and leverages the creation of in silico synthetic doublets to determine which cells in the input count matrix have gene expression that is best explained by the combination of distinct cell types in the matrix. Each patient was processed separately, cells with p-values <1e-7 were identified as doublets and removed. In total, ˜1,300 cells were removed.

Normalization and Batch effect correction. Filtered count matrices for each patient were median size normalized. To avoid numerical issues counts were multiplied by 10,000 and log transformed with pseudo count of 1 using SCANPY package (Subira et al., Role of flow cytometry immunophenotyping in the diagnosis of leptomeningeal carcinomatosis. Neuro Oncol 14, 43-52 (2012)). After normalization, count matrices were concatenated and batch effect corrected with mnnCorrect (Nemeth et al., IL-6 mediates hypoferremia of inflammation by inducing the synthesis of the iron regulatory hormone hepcidin. J Clin Invest 113, 1271-1276 (2004) and Yang et al., An iron delivery pathway mediated by a lipocalin. Mol Cell 10, 1045-1056 (2002)). As a reference, the patient with the most cells (patient E) was selected and to facilitate identification of mutual nearest neighbors the top 3,000 highly variable genes (HVGs) identified from all patients were used and opted for non-cosine normalized batch effect corrected output for downstream analyses.

Dimensionality reduction, Clustering, visualization and imputation. Using earlier selected top 3,000 HVGs, batch effect corrected gene expression matrix was decomposed using randomized principal component analysis (PCA). 12 principal components using the knee point (minimum radius of curvature in eigenvalues) were retained. The PCA-reduced matrix was clustered was using PhenoGraph resulting in 18 clusters. The same principal components were used to construct a k-nearest-neighbor (kNN) graph based on Euclidian distance. This kNN graph was used to generate UMAP projections using the SCANPY package. In addition, MAGIC was applied to the PCA-reduced matrix to denoise the data and impute missing values. Note, all UMAP plots show post-MAGIC expression values.

Gene signatures. Hallmark hypoxia, BIOCARTA_NFKB_PATHWAY, KEGG_JAK_STAT_SIGNALING_PATHWAY gene signatures were downloaded from MSigDB (http://software.broadinstitute.org/gsea/msigdb). Iron ion transport GO term GO:0006826 was obtained from Gene Ontology (http://geneontology.org/). Extensive list of pro-inflammatory cytokines was acquired from a recent publication. Full list of genes in the signatures is provided in Table 5. MAGIC imputed gene expression was used for calculating mean gene signature expression per cell. Next, mean gene signature expression was z-scored across the cells by scaling the expression that the mean would be equal to zero and unit standard deviation.

TABLE 3
Correlation between CSF LCN2, IL-6, Hepcidin and iron concentration

| | LCN2 (ng/mL) | | Hepcidin (ng/mL) | | IL-6 (ng/mL) | |
|---|---|---|---|---|---|---|
| | R² | P | R² | P | R² | P |
| Total Iron (μM) | 0.7208 | <0.0001 | 0.4534 | <0.0001 | 0.7261 | <0.0001 |
| LCN2 (ng/mL) | — | — | 0.7150 | <0.0001 | — | — |
| IL-6 (pg/mL) | 0.8271 | <0.0001 | 0.5711 | <0.0001 | — | — |

Differential expression of genes. Differentially expressed genes between PhenoGraph clusters were determined using MAST. MAST was run using default parameters with normalized counts (without log transformation, batch correction and before MAGIC) as the input. Genes with FDR (false discovery rate)-corrected P value <1×10-3 and absolute log 2(fold change)>0.3 are provided in Table 5, Table 4.

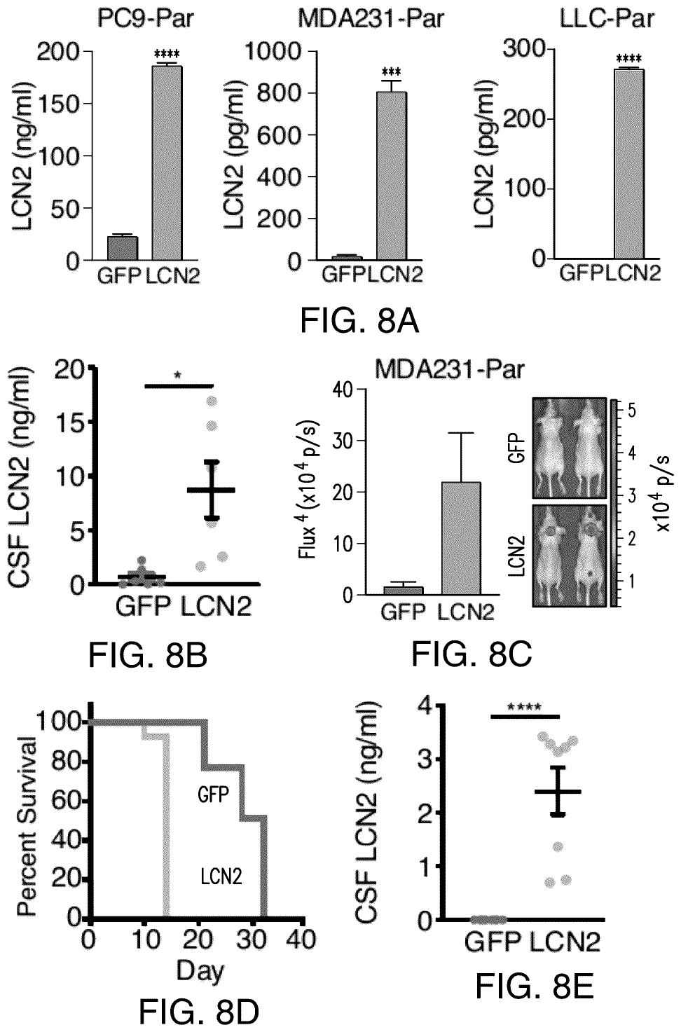

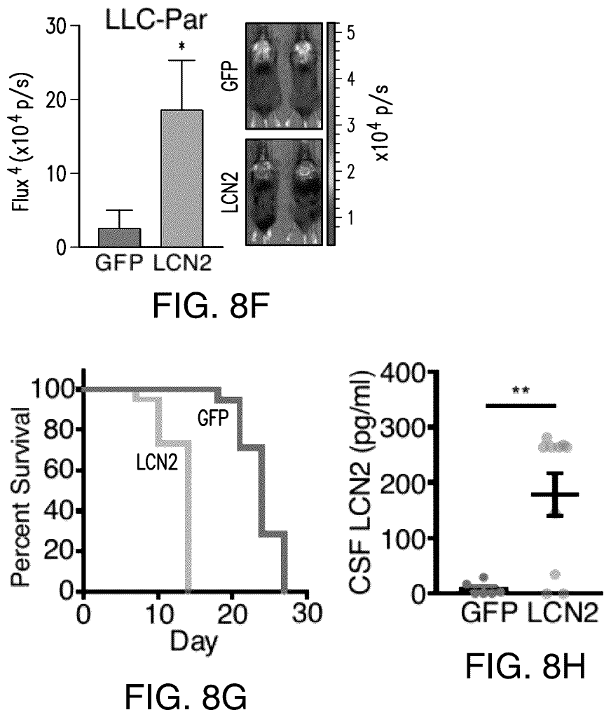

Annotation of cell types. Cell quality control steps described earlier are quite permissive and sometimes fail to eliminate all low-quality cells. These cells typically do not express any particular genes and frequently have low library size. In the dataset, after careful consideration, cluster 10 was identified as low-quality, dying cells and ultimately eliminated. Moreover, cluster 5 and 17 exhibited both T cell and myeloid lineage phenotypic profiles and were eliminated from the dataset. To facilitate annotation of remaining 15 clusters identified by PhenoGraph, MAGIC imputed gene expression of known marker genes (FIG. 8A) and MAST derived differentially expressed genes between PhenoGraph clusters were examined. Markers used to identify major cell types included MS4A1 and CD79A (B cells), IL3RA and CLEC4C (plasmacytoid DC), EPCAM and KRT18 (Cancer cells), CD3D and CD8A (CD8 T cells), CD3D, IL7R and CD4 (CD4 T cells), GNLY, NKG7, KLRB1 and NCAM1 (NK cells), CD14, CD68 and CST3 (Macrophages), FCGR3A, LYZ and CST3 (Monocytes), CST3 and FCER1A (conventional DC). In addition, the correspondence between PhenoGraph clusters and gene expression profiles from sorted bulk hematopoietic populations downloaded from the Dmap portal (http://portals.broadinstitute.org/dmap/home) were also inspected. Briefly, bulk samples were library-size normalized and pre-processed by mean centering. In the case of PhenoGraph clusters, MAGIC imputed gene expression was mean centered and the correlations were based on ˜2000 highly expressed genes. All cell typing methods used were in good agreement.

TABLE 4
Preclinical Model Characteristics

| | Median | | Spinal | |
|---|---|---|---|---|
| | Survival | Cranial | Cord | |
| PC9-LeptoM | (Days) | LM | LM | [Fe](μM) |
| Veh | 31.5 ± 3.6 | 8/8 (100%) | 8/8 (100%) | 16.28 ± 3.63 |
| D-Pen | 31.5 ± 3.5 | 8/8 (100%) | 8/8 (100%) | 15.13 ± 4.21 |
| DFO Early | 52 ± 7 | 3/8 (37.5%) | 0/8 (0%) | 5.63 ± 0.70 |
| DFO late | 40 ± 5 | 6/9 (66.7%) | 1/9 (11.1%) | 4.74 ± 0.38 |
| | Median | | Spinal | |
| MDA231- | Survival | Cranial | Cord | |
| LeptoM | (Days) | LM | LM | [Fe](μM) |
| Veh | 28 ± 7 | 9/9 (100%) | 9/9 (100%) | 40.69 ± 3.48 |
| D-Pen | 28 ± 7 | 9/9 (100%) | 9/9 (1001%) | 39.44 ± 2.43 |
| DFO Early | 54.5 ± 5.5 | 7/10 (70%) | 4/10 (40%) | 10.55 ± 1.31 |
| DFO late | 38 ± 7 | 7/9 (77.8%) | 1/8 (11.1%) | 9.19 ± 0.98 |

Diffusion component analysts. Diffusion maps were used as a nonlinear dimensionality reduction technique to find the major non-linear components of variation in earlier defined macrophage population. Diffusion components were computed using the Palantir Python package, which implements diffusion maps using the kernel as described in van Dijk et al., Cell, 174 716-729, e727 (2018). Eigengap was used to determine the number of significant components. Ranked gene list for each diffusion component was obtained by correlating non-imputed expression of each gene to a particular component and used as input for preranked Gene Set Enrichment Analysis (GSEA)(Subramanian et al., PNAS 102, 15545-15550 (2005)). In this case, Hallmark gene set supplemented with additional gene sets provided in Table 5 were used. GSEA analysis reports are provided in Table 5.

TABLE 5
List of Gene Signatures

| | | | | KEGG/JAK/ |
|---|---|---|---|---|
| | Proinflamation | | | STAT/ |
| Hallmark | (reference | Iron ion | BIOCARTA_NFKB_ | SIGNALING |
| hypoxia | https://doi.org/10.1016/ | transport | PATHWAY | PATHWAY |
| (MsigDB) | j.bbamcr.2014.05.014 ) | (GO:0006826) | (MsigDB) | (MsigDB) |
| ′ADM′, | ′IL1A′, | ′ABCB6′, | ′CHUK′, | ′AKT1′, |
| ′ADORA2B′, | ′IL1B′, | ′ARHGAP1′, | ′FADD′, | ′AKT2′, |
| ′AK4′, | ′IL1RN′, | ′ATP7A′, | ′IKBKB′, | ′AKT3′, |
| ′AKAP12′, | ′IL18′, | ′B2M′, | ′IKBKG′, | ′BCL2L1′, |
| ′ALDOA′, | ′IL33′ | ′CLTC′, | ′IL1A′, | ′CBL′, |
| ′ALDOB′, | ′IL36A′ | ′CP′, | ′IL1R1′, | ′CBLB′, |
| ′ALDOC′, | ′IL36B′, | ′CUBN′, | ′IRAK1′, | ′CBLC′, |
| ′AMPD3′, | ′IL36G′, | ′DNM2′, | ′MAP3K1′, | ′CCND1′, |
| ANGPTL4′, | ′IL36RN′, | ′FLVCR1′, | ′MAP3K14′, | ′CCND2′, |
| ′ANKZF1′, | ′IL37′, | ′FLVCR2′, | ′MAP3K7′, | ′CCND3′, |
| ′ANXA2′, | ′IL1F10′, | ′FTH1′, | ′MYD88′, | ′CISH′, |
| ′ATF3′, | ′IL6′, | ′FTMT′, | ′NFKB1′, | ′CLCF1′, |
| ′ATP7A′, | ′IL11′, | ′HAMP′, | ′NFKBIA′, | ′CNTF′, |
| ′B3GALT6′, | ′IL31′, | ′HAMP2′, | ′RELA′, | ′CNTFR′, |
| ′B4GALNT2′, | ′CNTF′, | ′HEPH′, | ′RIPK1′, | ′CREBBP′, |
| ′BCAN′, | ′CTF1′, | ′HEPHL1′, | ′TAB1′, | ′CRLF2′, |
| ′BCL2′, | ′LIF′, | ′HFE′, | ′TLR4′, | ′CSF2′, |
| ′BGN′, | ′SPP1′, | ′HPX′, | ′TNF′, | ′CSF2RA′, |
| ′BHLHE40′, | ′OSM′, | ′HRG′, | ′TNFAIP3′, | ′CSF2RB′, |
| ′BNIP3L′, | ′TNF′, | ′LCN2′, | ′TNFRSF1A′, | ′CSF3′, |
| ′BRS3′, | ′LTA′, | ′LMTK2′, | ′TNFRSF1B′, | ′CSF3R′, |
| ′BTG1′, | TNFSF13B′, | ′LRP2′, | ′TRADD′, | ′CSH1′, |
| ′CA12′, | ′TNFSF13′, | ′MELTF′, | ′TRAF6′ | ′CTF1′, |
| ′CASP6′, | ′IL17A′, | ′MMGT1′, | | ′EP300′, |
| ′CAV1′, | ′IL17B′, | ′MYO1B′, | | ′EPO′, |
| ′CCNG2′, | ′IL17C′, | ′NECTIN1′, | | ′EPOR′, |
| ′CCRN4L′, | ′IL17D′, | ′NOS1′, | | ′GH1′, |
| ′CDKN1A′, | ′IL17F′, | ′PICALM′, | | ′GH2′, |
| ′CDKN1B′, | ′IL25′, | ′RAB11B′, | | ′GHR′, |
| CDKN1C, | ′IL17E′, | REP15′, | | ′GRB2′, |
| ′CHST2′, | ′IFNA1′, | ′SCARA5′, | | ′IFNA1′, |
| ′CHST3′, | ′INFB1′, | ′SFXN1′, | | ′IFNA10′, |
| ′CITED2′, | ′IFNW1′, | ′SLC11A1′, | | ′IFNA13′, |
| ′COL5A1′, | ′IFNK′, | ′SLC11A2′, | | ′IFNA14′, |
| ′CP′, | ′LIMITIN′, | ′SLC22A17′, | | ′IFNA16′, |
| ′CSRP2′, | ′IFNG′, | ′SLC25A28′, | | ′IFNA17′, |
| CTGF, | ′IFNL1′, | ′SLC25A37′, | | ′IFNA2′, |
| ′CXCR4′, | ′IFNL2′, | ′SLC39A14′, | | ′IFNA21′, |
| ′CXCR7′, | ′IFNL3′ | ′SLC40A1′, | | ′IFNA4′, |
| ′CYR61′, | | ′SLC46A1′, | | ′IFNA5′, |
| ′DCN′, | | ′SLC48A1′, | | ′IFNA6′, |
| ′DDIT3′, | | ′SNX3′, | | ′IFNA7′, |
| ′DDIT4′, | | ′STEAP1′, | | ′IFNA8′, |
| ′DPYSL4′, | | ′STEAP2′, | | ′IFNAR1′, |
| ′DTNA′, | | ′STEAP3′, | | ′IFNAR2′, |
| ′DUSP1′, | | ′STEAP4′, | | ′IFNB1′, |
| ′EDN2′, | | ′TFR2′, | | ′IFNE′, |
| ′EFNA1′, | | ′TFRC′, | | ′IFNG′, |
| ′EFNA3′, | | ′TIMD2′, | | ′IFNGR1′, |
| EGFR, | | ′TRF′ | | ′IFNGR2′, |
| ′ENO1′, | | | | ′IFNK′, |
| ′ENO2′, | | | | ′IFNW1′, |
| ′ENO3′, | | | | ′IL10′, |
| ′ERO1L′, | | | | ′IL10RA′, |
| ′ERRFI1′, | | | | ′IL10RB′, |
| ′ETS1′, | | | | ′IL11′, |
| ′EXT1′, | | | | ′IL11RA′, |
| ′F3′, | | | | ′IL12A′, |
| ′FAM162A′, | | | | ′IL12B′, |
| ′FBP1′, | | | | ′IL12RB1′, |
| ′FOS′, | | | | ′IL12RB2′, |
| FOSL2, | | | | ′IL13′, |
| ′FOXO3′, | | | | IL13RA1′, |
| ′GAA′, | | | | ′IL13RA2′, |
| ′GALK1′, | | | | ′IL15′, |
| ′GAPDH′, | | | | ′IL15RA′, |
| ′GAPDHS′, | | | | ′IL19′, |
| ′GBE1′, | | | | ′IL2′, |
| ′GCK′, | | | | ′IL20′, |
| GCNT2′, | | | | ′IL20RA′, |
| ′GLRX′, | | | | ′IL20RB′, |
| ′GPC1′, | | | | ′IL21′, |
| ′GPC3′, | | | | ′IL21R′, |
| ′GPC4′, | | | | ′IL22′, |
| ′GP1′, | | | | ′IL22RA1′, |
| ′GRHPR′, | | | | ′IL22RA2′, |
| ′GYS1′, | | | | ′IL23A′, |
| ′HAS1′, | | | | ′IL23R′, |
| ′HDLBP′, | | | | ′IL24′, |
| ′HEXA′, | | | | ′IL26′, |
| HK1, | | | | ′IL28A′, |
| ′HK2′, | | | | ′IL28B′, |
| ′HMOX1′, | | | | ′IL28RA′, |
| ′HOXB9′, | | | | ′IL29′, |
| ′HS3ST1′, | | | | ′IL2RA′, |
| ′HSPA5′, | | | | ′IL2RB′, |
| IDS, | | | | ′IL2RG′, |
| ′IER3′, | | | | ′IL3′, |
| ′IGFBP1′, | | | | ′IL3RA′, |
| ′IGFBP3′, | | | | ′IL4′, |
| IL6 | | | | ′IL4R′. |
| ′ILVBL′, | | | | ′IL5′, |
| ′INHA′, | | | | ′IL5RA′ |
| ′IRS2′, | | | | ′IL6′, |
| ′ISG20′, | | | | ′IL6R′, |
| ′JMJD6′, | | | | ′IL6ST′, |
| JUN, | | | | ′IL7′, |
| ′KDELR3′, | | | | ′IL7R′, |
| ′KDM3A′, | | | | ′IL9′, |
| ′KIF5A′, | | | | ′IL9R′. |
| ′KLF6′, | | | | ′IRF9′, |
| ′KLF7′, | | | | ′JAK1′, |
| KLHL24′, | | | | ′JAK2′, |
| ′LALBA′, | | | | ′JAK3′, |
| LARGE′, | | | | ′LEP′, |
| ′LDHA′, | | | | ′LEPR′, |
| ′LDHC′, | | | | ′LIF′, |
| ′LOX′, | | | | ′LIFR′, |
| ′LXN′, | | | | ′MPL′, |
| ′MAFF′, | | | | ′MYC′, |
| ′MAP3K1′, | | | | ′OSM′, |
| ′MIF′, | | | | ′OSMR′, |
| ′MT1E′, | | | | ′PIAS1′, |
| ′MT2A′, | | | | ′PIAS2′, |
| ′MXI1′, | | | | ′PIAS3′, |
| ′MYH9′, | | | | ′PIAS4′, |
| ′NAGK′, | | | | ′PIK3CA′, |
| ′NCAN′, | | | | ′PIK3CB′, |
| ′NDRG1′, | | | | PIK3CD′, |
| ′NDST1′, | | | | ′PIK3CG′, |
| ′NDST2′, | | | | ′PIK3R1′, |
| ′NEDD4L′, | | | | ′PIK3R2′, |
| ′NFIL3′, | | | | ′PIK3R3′, |
| ′NR3C1′, | | | | ′PIK3R5′, |
| ′P4HA1′, | | | | ′PIM1′, |
| ′P4HA2′, | | | | ′PRL′, |
| PAM, | | | | ′PRLR′, |
| ′PCK1′, | | | | ′PTPN11′, |
| ′PDGFB′, | | | | ′PTPN6′, |
| ′PDK1′, | | | | ′SOCS1′, |
| ′PDK3′, | | | | ′SOCS2′, |
| ′PFKFB3′, | | | | ′SOCS3′, |
| ′PFKL′, | | | | ′SOCS4′, |
| ′PFKP′, | | | | ′SOCS5′, |
| ′PGAM2′, | | | | ′SOCS7, |
| ′PGF′, | | | | ′SOS1′, |
| ′PGK1′, | | | | ′SOS2′, |
| ′PGM1′, | | | | ′SPRED1′, |
| ′PGM2′, | | | | ′SPRED2′, |
| ′PHKG1′, | | | | ′SPRY1′, |
| ′PIM1′, | | | | ′SPRY2′, |
| ′PKLR′, | | | | ′SPRY3′, |
| ′PKP1′, | | | | ′SPRY4′, |
| ′PLAC8′, | | | | ′STAM′, |
| ′PLAUR′, | | | | ′STAM2′, |
| ′PLIN2′, | | | | ′STAT1′, |
| ′PNRC1′, | | | | ′STAT2′, |
| ′PPARGC1A′, | | | | ′STAT3′, |
| ′PPFIA4′, | | | | ′STAT4′, |
| ′PPP1R15A′, | | | | ′STAT5A′, |
| ′PPP1R3C′, | | | | ′STAT5B′, |
| ′PRDX5′, | | | | ′STAT6′, |
| ′PRKCA′, | | | | ′TPO′, |
| ′PRKCDBP′, | | | | ′TSLP′, |
| ′PTRF′, | | | | ′TYK2′ |
| ′PYGM′, | | | | |
| ′RBPJ′, | | | | |
| ′RORA′, | | | | |
| ′RRAGD′, | | | | |
| ′S100A4′, | | | | |
| SAP30′, | | | | |
| ′SCARB1′, | | | | |
| ′SDC2′, | | | | |
| ′SDC3′, | | | | |
| ′SDC4′, | | | | |
| ′SELENBP1′, | | | | |
| ′SERPINE1′, | | | | |
| ′SIAH2′, | | | | |
| ′SLC25A1′, | | | | |
| ′SLC2A1′, | | | | |
| ′SLC2A3′, | | | | |
| ′SLC2A5′, | | | | |
| ′SLC37A4′, | | | | |
| ′SLC6A6′, | | | | |
| ′SRPX′, | | | | |
| ′STBD1′, | | | | |
| ′STC1′, | | | | |
| ′STC2′, | | | | |
| ′SULT2B1′, | | | | |
| ′TES′, | | | | |
| ′TGFB3′, | | | | |
| ′TGFBI′, | | | | |
| ′TGM2′, | | | | |
| ′TIPARP′, | | | | |
| ′TKTL1′, | | | | |
| ′TMEM45A′, | | | | |
| ′TNFAIP3′, | | | | |
| ′TPBG′, | | | | |
| ′TPD52′, | | | | |
| ′TPI1′, | | | | |
| TPST2′, | | | | |
| ′UGP2′, | | | | |
| ′VEGFA′, | | | | |
| ′VHL′, | | | | |
| ′VLDLR′, | | | | |
| ′WISP2′, | | | | |
| ′WSB1′, | | | | |
| ′XPNPEP1′, | | | | |
| ′ZFP36′, | | | | |
| ′ZNF292′ | | | | |

In vivo leptomeningeal metastasis assays. To model leptomeningeal metastasis with a known consistent disease burden, 2×10³ PC9, 2×10³ MDA231, or 1×10³ LLC Parental or LeptoM cells suspended in 10 μl PBS were injected into the cisterna magna of anesthetized mice. This procedure was tolerated well by the mice. Tumor progression was monitored by bioluminescence imaging (BLI) every 7 days after tumor cell inoculation, and mouse condition was monitored daily. When the tumor cells encompassed the entire CNS, or when neurological impairment became apparent, CSF was collected as described above, and mice were euthanized. Brains and spinal cord were collected in tissue fixative and embedded for preservation or histological and morphometric analysis. The role of LCN2 in cancer progression at multiple metastatic sites was also assessed. Tumor burden was monitored and quantified non-invasively after tumor cell inoculation either by BLI signal using the IVIS Spectrum Xenogen (Caliper Life Sciences) machine or with calibrated digital caliper. To track cancer cell growth after hematogenous dissemination, 5×10⁴ PC9-LeptoM cells (shCtl, shLCN2-1 and shLCN2-2) suspended in 50 μL PBS were injected into the left heart ventricle. Tumor burden was monitored every 3 days with BLI. To measure cancer cell growth in the lungs, 2.5×10⁴ PC9-LeptoM cells (diluted 1:1 in Growth factor-reduced Matrigel, #354230) were injected into the left lung through the rib cage. Tumor burden was monitored every 3 days with BLI. To assess cancer cell growth after subcutaneous inoculation, 1×10⁶ PC9-LeptoM cells (diluted 1:1 in growth factor-reduced Matrigel) were injected under the flank skin of the mouse, and tumor burden was monitored every 3 days with calibrated digital caliper. To introduce cells into the mammary fat pad, 5×10⁴ MDA-231-LeptoM cells (diluted 1:1 in growth factor-reduced Matrigel) were injected into the fourth inguinal mammary fat pad; tumor burden was measured every 3 days with calibrated digital caliper. To test a hypoxic environment other than the leptomeninges, 5×10⁶ cells, diluted in 200 uL PBS, were injected intraperitoneally. Tumor burden was monitored 7 days after inoculation by BLI. Exact numbers for each experimental series are included in the relevant Figure legends.

Mouse CSF collection and Intrathecal drug administration. As previously described (1), anesthetized mice were positioned prone over a 15 mL conical tube to induce cervical flexion. The occiput was palpated to identify the cisterna magna, and a 1 mL TB syringe with a 30G beveled needle was inserted 4 mm deep between the occiput and C1 at an angle of approximately 45 degrees. Depending upon experimental design, either 10 μL of CSF was withdrawn, or 10 μL of treatment solution was injected: 10 mM Deferoxamine mesylate (DFO, Sigma-Aldrich, D0160000), 10 mM D-penicillamine (D-Pen, Sigma-Aldrich, P4875), 5 mg/mL Transferrin (Invitria, 777TRF029) or vehicle (PBS) was administered into the cisterna magna every 3 days. At indicated time points, CSF was collected for assays as described below.

Induction-Coupled Plasma Mass Spectrometry. CSF and total cellular iron were measured by induction coupled plasma mass spectrometry (ICP-MS; Agilent 7900) equipped with integrated sample introduction system (ISIS3) in high energy helium mode (10 ml/min; 1 point/peak, 4 replicates, 25 sweeps/replicate). The method was linear between 3-1000 ng/mL. Cell pellets (200,000-1,000,000) were digested overnight in 1004 of tetramethylammonium hydroxide. The cell pellet digestate and CSF (50 uL) were diluted 1:80 in diluent (4% 1-butanol, 1% TMAH, 0.01% Triton X-100, 0.01% ammonium pyrrolidinedithiocarbamate) and quantified using the iron isotopes 56 and 57 relative to a 7-point calibration and germanium as an internal standard.

Tissue preparation and immunostaining. Tissues were collected as described above. All tissue was fixed in 10% neutral buffered formalin (Sigma-Aldrich, HT501128) for 18 hr at 4° C. The brains were then sliced into 4 coronal sections, placed in a plastic cartridge, soaked in 70% ethanol, and stored at 4° C. The spinal cord samples were decalcified in 0.5 M EDTA [pH 8.0] for 4 days. Excess tissue was cut from around the spinal column, and the tissue was placed in a plastic cartridge and soaked for 4 days in 0.5 M EDTA. The tissue was rinsed for 1 hr with deionized water, and fixed with 10% neutral buffered formalin for 18 hr. The cartridges were then soaked in 70% ethanol and stored at 4° C. The tissue was processed at the MSK Molecular Cytology core facility. All tissues were embedded in paraffin, and 5 μm for staining. Antigen unmasking was done by incubation of sections in Target Retrieval Solution (Dako, S1699) and steamed for 30 min. The primary antibodies were used as follows: rabbit anti-SLC22A17 (1:200), goat anti-LCN2 (1:50), goat anti-Transferrin (1:100), mouse anti-Ferritin (1:100), mouse anti-Hepcidin (1:100), rabbit anti-Iba-1 (1:1000), sheep anti-Cytokeratin (1:800), mouse anti-CD68 (1:100), mouse anti-HIF-1α (1:1000), rabbit anti-HIF-2a (1:1000). For indirect immunofluorescence (IF), secondary antibodies (1:800) incubated for 30 min. Sections were washed three times in wash buffer (DAKO, S300685), and counter-stained with the nuclear dye DAPI (1:1000) for 5 min, followed by two washes in wash buffer. Sections were transferred onto slides and mounted using Fluoro-Gel mount (Electron Microscopy Sciences, 17985). For immunohistochemistry (IHC), ImmPRESS™ HRP reagent kits (Vector Laboratories, MP-7405, MP-7402, MP-7401) were used as secondary antibody. The slides were incubated with liquid DAB+ Substrate Chromogen System (Dako, K346811) for 2 min, washed with distilled water twice, counterstained with hematoxylin (Dako, S3309). Images were acquired with a Panoramic MIDI scanner (3DHISTECH Ltd.) and analyzed with CaseViewer (3DHISTECH Ltd.), ImageJ (US National Institutes of Health) and Photoshop (Adobe Systems).

Immunocytochemistry. 1×10⁴ cells were seed in Chamber slide (Lab-Tek) overnight and fixed with 4% paraformaldehyde. The slides were blocked at room temperature for 1 hr in TBS containing 10% normal donkey serum (Jackson ImmunoResearch) and 0.1% Triton X-100 (Sigma-Aldrich, T8787). Samples were incubated overnight at 4° C. with primary antibodies: goat anti-LCN2 (1:50), rabbit anti-SLC22A17 (1:200). After 3 washes with TBST, samples were incubated for 1 hr with the appropriate secondary antibodies. Cell nuclei were stained using DAPI (Life Technologies). Images were acquired with a confocal microscope (Olympus, FV1000), and analyzed with ImageJ (NIH), and Photoshop (Adobe Systems).

Hypoxia detection in vitro and in vivo. To detect hypoxia during LM, a dual luciferase assay system was first generated by transfecting pNL3.2-HRE-Nluc vector into LeptoM cells expressing Firefly luciferase (Fluc) leading to stable expression of dual reporter Nluc and Fluc below 2% 02. Hypoxia was modeled in vitro by culture in at 0.5% O₂ and 37° C. for up to 72 hr. Cells were placed in the hypoxic incubator (CoxLab) and removed at various time points to perform the luminescence intensity of cells with hypoxic conditions. Apoptosis (Promega, G8091) assayed after 48 hr. In vivo, 2×10³ HRE-expressing cancer cells were inoculated into mice by intrathecal injection within passage 8. Hypoxia and tumor burden were identified by BLI image from different time points and normalized to day 0.

Flow cytometry analysis and sorting. For cytometric bead assay, CSF from control mice or mice bearing LLC LeptoM cells was collected on day 14 as described above. Levels of cytokines were measured using LEGENDplex Mouse Inflammation Panel (13-plex; 740446, Biolegend) as recommended by manufacturer in technical duplicates. Data was processed using LEGENDPlex Software (Biolegend). For flow cytometric analysis of CSF and meningeal cells, mice were transcardially perfused though left ventricle with saline using standard procedures (16). Then, CSF was collected by cisternal tap as described above. Next, cranial vault was removed, brain was collected and basilar meninges were washed with PBS. Surface of the brain and ventricular spaces were washed with PBS. CSF, cavity and brain washouts were pooled and processed for staining. For analysis of peripheral leukocytes, blood was collected from deeply anesthesized mice using cardiac puncture. Spleen was collected from perfused animals, minced, and mechanically dissociated using GentleMACS (Miltenyi Biotec). All samples were filtered through 30 micron mesh before staining. Blocking of unspecific binding sites was performed with 10% rat serum (Sigma-Aldrich, R9759) in PBS for 10 min, followed by surface staining for 15 min. Primary antibodies were diluted in Brilliant Stain Buffer (BD, 563794), supplemented with 5% rat serum. LIVE/DEAD Green/Violet/FarRed Dead Cell Stain kits (Life Technologies, L34969, L34963, L34973, respectively) was used as viability stain. Red blood cells were lysed with 1× eBioscience RBC Lysis Buffer for 5 min (Invitrogen, 00-4300-54). Cells were fixed with IC Fixation Buffer for 20 min (Invitrogen, 00-8222-49), permeabilized in 1× Permeabilization Buffer for 1 h (Invitrogen, 00-8333-56). To sort macrophages from the mouse CSF and meninges, cells were labeled with antibodies against CD45, CD11b and F4/80, as described above. Dead cells were excluded using DAPI (Molecular Probes, D1306). For functional analysis, cells were collected from cranial cavity from saline-perfused mice as described above. Phagocytic activity and respiratory burst were analyzed using E. coli Phagocytosis Assay Kit (FITC conjugate, Cayman Chemicals, 601370) and CellRox Green Flow Cytometry (Thermo Fisher, C10492), as recommended. Cells were incubated for 1 hr at 37° C. without exogenous stimuli in the supplied buffer. All further incubations were performed on ice. Extracellular and viability stains and red blood cell lysis followed.

Mouse cell populations were identified as follows: CD45+ CD3+T-lymphocytes, CD45+ CD11b+ Ly6G+ neutrophils, CD45+ Nk1.1+ NK cells, CD45+ CD11b+ (CD14+)Ly6Chigh monocytes, CD45+ CD11b+ (CD14+)Ly6C+ F4/80+ macrophage, CD45+ CD11b+ (CD14−) other myeloid cells, CD45− cancer cells. For analysis of human CSF cells, CSF was processed as described above and cellular fractions were cryopreserved using serum-free cryopreservant (GC Lymphotec, Bambanker BB01) and stored at −80° C. until further use. Samples were then quickly thawed in the 37° C. liquid bath and washed with PBS. Blocking of unspecific binding sites was performed with 5% rat and 5% mouse serum (Sigma-Aldrich, M5905) in PBS for 10 min, followed by surface stain for 20 min. Primary antibodies were diluted in Brilliant Stain Buffer (BD Biosciences, 563974), supplemented with 2.5% rat and 2.5% mouse serum. LIVE/DEAD Violet Dead Cell Stain kit was used as viability probe. Red blood cell lysis was not performed. Cells were fixed after surface stain with IC Fixation Buffer for 20 min, permeabilized and then stained for intracellular antigens (pan-Cytokeratin, CD68 and LCN2) in 1× Permeabilization Buffer for 40 min. After all washing steps, cells were spun at 600 g. To sort cells from human CSF, cells were labeled with antibodies against CD45, CD14, CD16, CD11b, CD64 and CD68, as described above. Dead cells were excluded using LIVE/DEAD Violet Dead Cell Stain kit. Human cell populations were identified as follows: CD45+ CD3+T-lymphocytes, CD45+ CD15+ neutrophils, CD45+ CD56+ NK cells, CD45+ CD11b+ CD14+ CD16-monocytes, CD45+ CD11b+ CD14+ CD16+ macrophage, CD45+ CD11b+ CD14− other myeloid cells, CD45−pan-Cytokeratin+ cancer cells. Data acquisition was performed on BD LSR Fortessa (BD) and FACSAria SORP Cell Sorter (BD). Acquired FCS files were exported and analyzed using FlowJo Software (v10.6.1, Tree Star).

Cytospin. Air-dried cytospin samples were prepared from patient CSF as previously described (1). Samples were fixed in 4% PFA for 10 min at room temperature, rinsed in PBS+1% BSA 3×5 min prior to Geimsa stain. Cell populations FAC-sorted from human CSF (previously fixed during antibody staining); were spun onto a slide, air-dried and stained with Giemsa. For immunostaining of mouse CSF, primary antibodies are incubated overnight and secondary antibodies (1:800) incubated for 30 min. Sections were counter-stained with the nuclear dye DAPI (1:1000) for 5 min, followed by washes twice in wash buffer.

Co-culture system of primary macrophages with cancer cells. Primary macrophage was isolated as described previously. In brief, spleen or CSF-derived macrophages (CD45+ CD11b+ F4/80+) were counted and suspended in 2% FBS RPMI 1640 media and 0.5 mM L-glutamine. 1×10⁵ primary macrophages were seeded in one well of 6-well plate (Corning, 353046). When primary macrophages reached ˜70% confluence (˜3 days after the isolation), the supernatant from primary microphages was collected and added to 1×10⁵ cancer cells. Fresh supernatant from primary macrophages was replenished in cancer cells every 3 days. After 12 days (4 rounds), the supernatant was removed, cancer cells were washed with PBS and cultured in serum-free RPMI media for 2 days. LCN2 expression in cancer cells was detected by ELISA. For measuring LCN2 mRNA in macrophages co-culture with cancer cells, 1×10⁵ cancer cells were seed the upper chamber (0.4 μm pore, Corning, USA) and 1×10⁵ primary macrophages were seed to the bottom chamber. After maintaining co-cultures for 14 days, macrophages and cancer cells were isolated and LCN2 expression was detected by qPCR.

Bulk transcriptomic analysis. After knockdown of LCN2 in PC9-LeptoM and MDA231-LeptoM, total RNA was extracted using the Rneasy Plus Mini kit (Qiagen). After RiboGreen quantification and quality control by Agilent BioAnalyzer, 500 ng of total RNA underwent polyA selection and TruSeq library preparation according to instructions provided by Illumina (TruSeq Stranded mRNA LT Kit, catalog #RS-122-2102), with 8 cycles of PCR. Samples were barcoded and run on a HiSeq 4000 in a 50 bp/50 bp paired end run, using the HiSeq 3000/4000 SBS Kit (Illumina). An average of 49 million paired reads was generated per sample. Reads from generated FASTQ files were quality checked, trimmed with CutAdapr v1.6, and mapped to the human reference genome (hg19) using STAR2.5.0.a. The expression count matrix of uniquely mapped reads was computed with Htseq v0.5.3. Differential gene expression analysis was performed with DESeq2 v1.22.1 pipeline in Bioconductor. Single-cell and bulk RNA-sequencing data have been deposited to NCBI GEO as GSE150681 SuperSeries. To compare gene expression among Par, BrM and LeptoM in LLC cells from bulk RNA sequencing data (GEO number GSE83132 in NCBI), previously generated transcriptomic data (Boire et al., Cell 168, 1101-1113 e1113 (2017)) was used to identify candidate genes involved in cytokines and iron transport. Gene set enrichment analysis (GSEA) was performed as described previously (Mootha et al., Nat Genet 34, 267-273 (2003))) (http://software.broadinstitute.org/gsea/index.jsp). To analyze transcriptional data from PC9-Par and PC9-LeptoM, the metabolism database and ion transport was used to create a matrix in which columns represented enrichment score (ES) of each gene set and rows represented metabolic and iron transport related gene sets. The biological replicates were averaged and filtered this matrix by P value <0.05.

Quantitative real-time PCR. Total RNA was isolated from samples with the Qiagen Rneasy Plus Mini kit according to standard protocol. RNA concentrations were quantified using a NanoDrop machine. 2 μg of RNA was used for cDNA synthesis with the Superscript IV VILO Mastermix kit (Thermo Fisher Scientific, 11766050). Relative qPCR analysis incorporated one housekeeping gene, ACTB. Quantitative PCR (qPCR) analysis to measure SLC22A17, MC4R and LRP2 message expression was determined by standard curves of dilutions with the cDNA samples. qPCR analysis of gene expression was performed on the ViiA 7 system (Applied Biosystems) with Power SYBR green PCR Master Mix (Applied Biosystems, 43-687-02).

Secreted protein detection. Human LCN2 (Abcam, ab215541), Hepcidin (R&D, DHP250), Transferrin (Abcam, ab187391), Ferritin (LSBio, LS-F428-1), IL-6 (R&D, D6050), IL-8 (R&D, D8000C), IL-1β (R&D, DLB50), TNF expression (Abcam, ab181421) in CSF patient samples or cell culture supernatants were measured using ELISA kits according to manufacturer's instructions. Mouse LCN2 (Abcam, ab119601), Hepcidin (LSBio, LS-F11620-1), Transferrin (LSBio, LS-F9542-1), Ferritin (LSBio, LS-F36489-1) ELISA kits were used in mouse CSF samples or cell culture supernatants. To detect secretion of LCN2 after cytokine stimulation, rIL-6 (R&D, 206-IL in human, 406-ML in mouse, 50 ng/μL), rIL-8 (R&D, 208-IL, 50 ng/μL) and rIL-1β (R&D, 201-LB in human, 401-ML in mouse, 10 ng/μL) and rTNF (R&D, 201-TA in human, 410-MT in mouse, 10 ng/μL) were added to the culture media for 48 hr prior to LCN2 ELISA. To block IL-6, IL-8 and IFN-γ in human CSF, antibodies neutralizing IL-6 (R&D, MAB206-100), IL-8 (R&D, MDA208-100) and IFN-γ (R&D) were incubated with CSF for 24 hr. ELISA assay for LCN2 was performed at 48 hr after changing media.

Western blot analysis. At least 1×10⁶ cells were collected by centrifugation 14,000×g at 4° C. and washed twice with ice-cold PBS. Ice-cold RIPA buffer (Thermo Fisher, 89901) containing fresh protease and phosphatase inhibitor cocktail (Thermo Fisher) was added to the cells for 20 min. After centrifugation at 14,000×g for 10 min at 4° C., cell lysate protein concentrations were determined using the BCA Protein Assay KIT (Thermo Fisher Scientific). 20 μg of protein samples were electrophoresed, transferred and detected using the following primary antibodies, as indicated: HIF-1α (1:1000), HIF-2α (1:1000), Transferrin (1:500) and mouse anti-β-actin (1:2000). The anti-rabbit, anti-mouse, anti-Goat (1:10000) conjugated with horseradish peroxidase (Sigma-Aldrich) were used as secondary antibodies, and immunoreactive signal was detected by enhanced chemiluminescence (ECL) (Thermo Fisher, 32109). Blots were imaged using LAS4000 luminescence imager.

Cell Viability Assay. 3×10³ cells per well of a 96-well plate were cultured in DMEM with 2% FBS in normal oxygen incubator and hypoxic incubator. The CellTiter-Glo Luminescent Cell Viability Assay (Promega G9241) was used to quantify viable cells in culture according to the manufacturer's protocols. For drug treatment in vitro, cells were treated by DFO (Sigma-Aldrich, D0160000) and D-Pen (Sigma-Aldrich, P4875) at final concentrations of 0 μM, 1 μM, 10 μM, or 100 μM. The Caspase-Glo 3/7 Assay system (Promega, G8091) was used to detect cell apoptosis over a period of up to 48 hr. 3×10³ cells were cultured per well of a 96-well plate in DMEM with 2% FBS.

Iron Assay. Cells were cultured in RPMI1640 with 2% FBS in normoxic and/or hypoxic incubator and washed once with ice-cold PBS prior to assay. The Iron Assay Kit (Sigma-Aldrich, MAK025) was used to quantify total iron in the intracellular contents and culture supernatant following the manufacturer's protocols. For iron competition assay between cancer cells and macrophages, cells were starved for 12 hr in FBS-free RPMI media and then incubated with various concentrations of Iron(III) nitrate (0, 0.05, 0.5 or 5 μM) and 3 mM of 2,5-Dihydroxybenzoic acid (2.5-DHBA, Sigma-Aldrich, 149357). For chelation assay in vitro, various concentrations of DFO or D-Pen (0, 1, 10 or 100 μM), or 100 ng/mL recombinant LCN2 (R&D, 1757-LC) and 100 μg/mL holo-Tf (Invitria, 777TRF029) were added to media as indicated. For LCN2 binding iron assay, 200 μL aliquots of patient CSF samples were divided equally. 100 μl was used for total iron assay [TI] following the previous protocol. The remaining 100 μl received 10 μl 0.2 mg/mL LCN2 antibody 4° C. for 3 hr. This was followed by 90 μL Protein A/G resin (Thermo, 53135) to bind the antigen-antibody complex for 2 hr at room temperature. Beads were collected by centrifugation for 3 min at 2500×g the supernatant was subjected to iron assay [SI]. Non-LCN2-bound iron [NLBI]=2×[SI], LCN2-bound iron [LBI]=[TI]−[NLBI].

Data presentation. For single-cell and bulk RNA-Seq analysis, please refer to above sections. All other statistical analysis and Figure plotting was performed using GraphPad Prism 8.00 software. Specific statistical tests employed for each experimental design are indicated in the corresponding Figure legends. Assessments with p<0.05 were considered significant. Exact value of N is annotated in each Figure legend. Values reported are means±standard error of the mean (SEM) or standard deviation (SD) as appropriate.

Results:

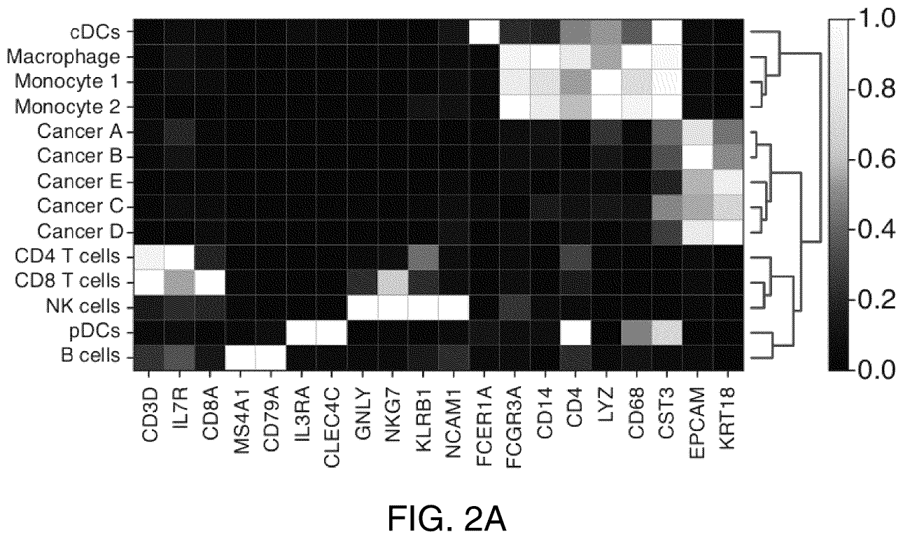

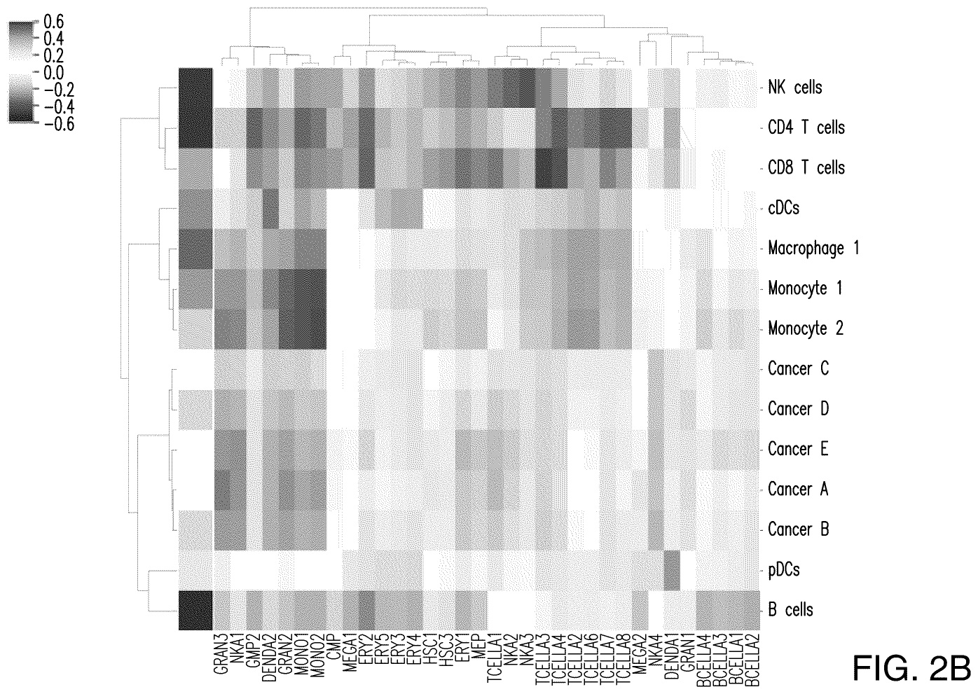

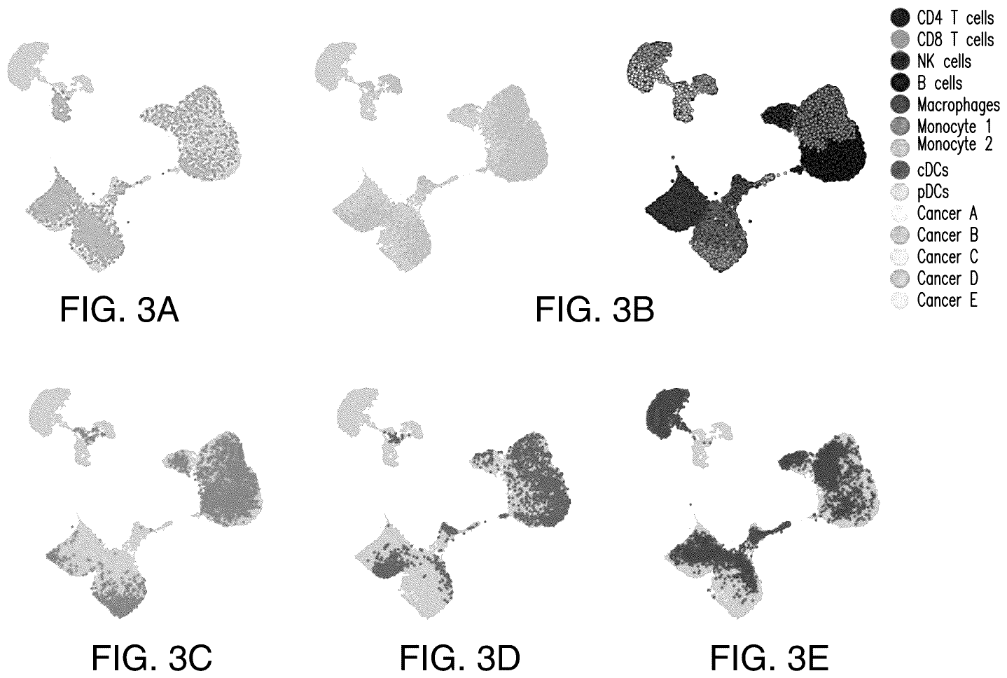

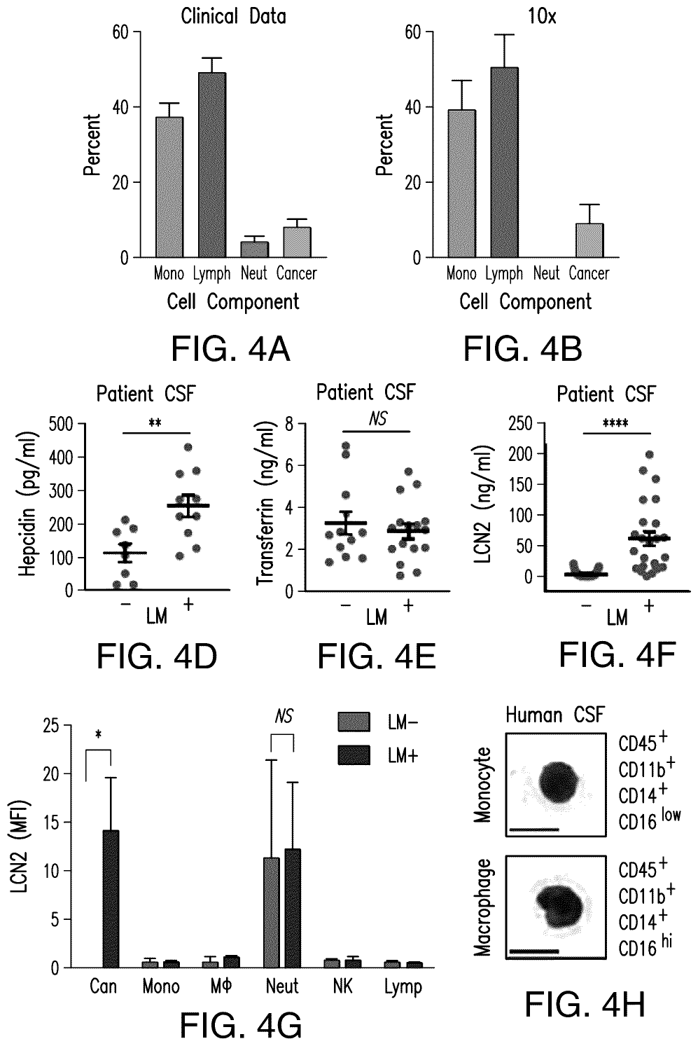

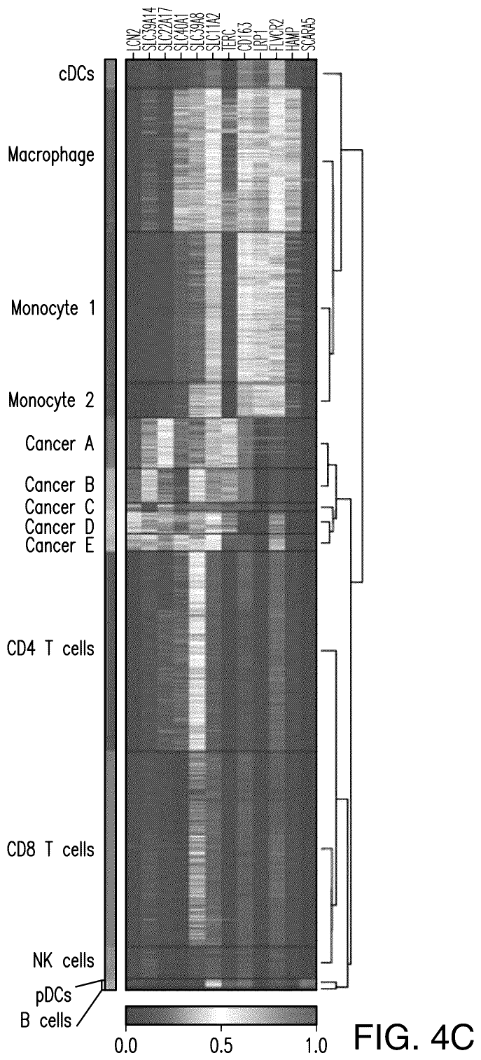

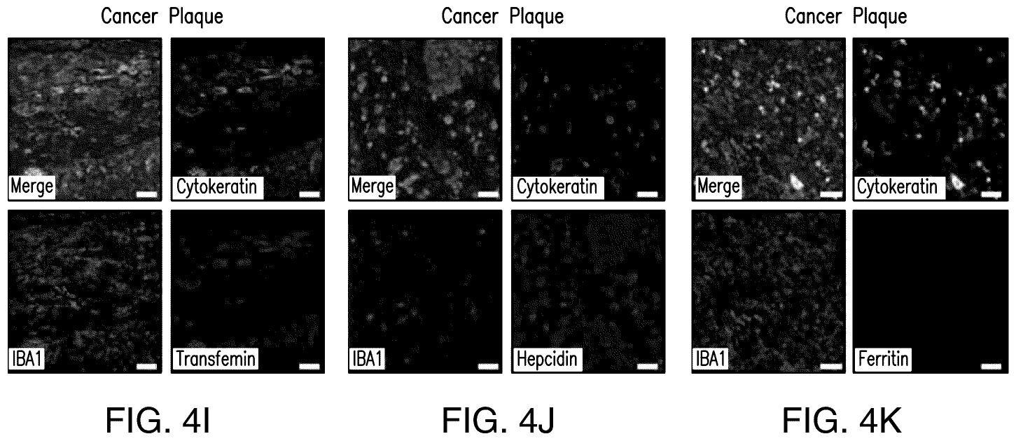

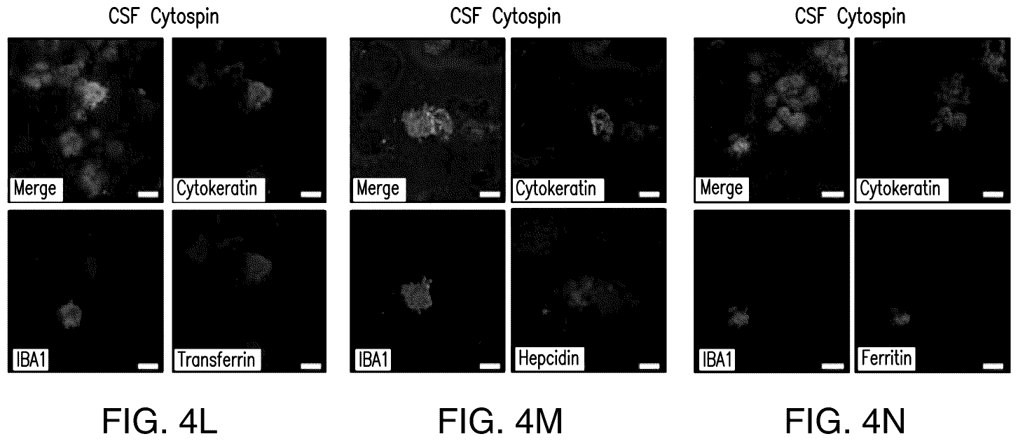

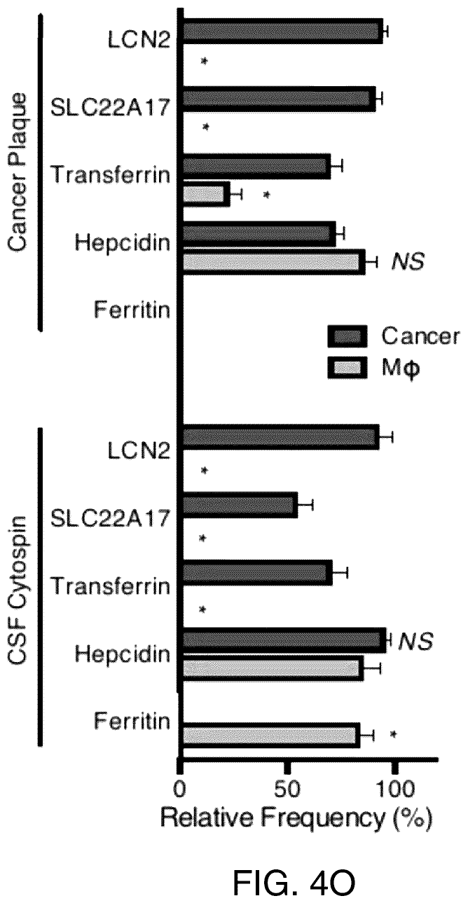

Cancer cells within human spinal fluid express LCN2 and SLC22A17. Cancer cells in the CSF disseminate throughout the central nervous system. Within this compartment they are vastly outnumbered by immune cells, primarily macrophages and lymphocytes (FIG. 1A)(4). To explore cancer and immune cell responses to the nutritionally sparse CSF, single-cell RNA sequencing (scRNA-Seq) was applied to cellular material collected from the CSF of five patients with LM (FIG. 1B, FIG. 2 and FIG. 3). LM was secondary to breast cancer primaries in three patients and to non-small cell lung cancer primaries in two patients (Table 1). After scRNA-Seq, the proportions of each cell type within the CSF remained consistent with those identified by clinical analysis (FIG. 4A to B).

It was found that all CSF cells showed upregulated expression of iron transport genes, consistent with functional iron deficiency within this anatomic space (FIG. 4C). Hepcidin, a protein implicated in both inflammation and iron deficiency, was detected at higher concentrations in the CSF of cancer patients harboring LM, compared with those without LM (FIG. 4D). In contrast, CSF levels of the ubiquitous iron transporter, transferrin, were equivalent in the two groups of patients (FIG. 4E). It was found that immune cells expressed canonical iron transporter transcripts, but cancer cells expressed a diverse array of genes associated with iron binding and transport (FIG. 4C). Of these, transcripts for a single iron-binding and receptor pair were expressed exclusively within the cancer cell population in all patients: lipocalin-2 (LCN2) and solute carrier family 22 member 17 (SLC22A17) (FIG. 1C to D). Protein expression corresponding to these single cell transcriptional data was confirmed by ELISA and flow cytometry of human CSF (FIG. 4F to H). Finally, in autopsy tissues, LCN2 and SLC22A17 protein expression was detected by immunofluorescence in the cancer cell population, and not in the macrophage/monocyte populations (FIG. 1E to H, and FIGS. 4I to O).

TABLE 1
Patient Characteristics

| | | | Time to | Survival | CSF | CSF | CSF |
|---|---|---|---|---|---|---|---|
| Patient | Primary | | LM Dx | Post LM | Protein | Glucose | WBC |
| ID | Tumor | Subtype | (Mos.) | Dx (Mos.) | (mg/dL) | (mg/dL) | (μL) |
| A | Lung | NSCLC | 45 | 20 | 36 | 68 | 1 |
| B | Breast | IDC | 26 | 13 | 63 | 49 | 6 |
| C | Breast | ILC | 88 | 10 | 707 | 36 | 2 |
| D | Lung | NSCLC | 42 | 16 | 46 | 32 | 8 |
| E | Breast | IDC | 57 | 13 | 66 | 23 | 3 |

LCN2, also known as neutrophil gelatinase-associated lipocalin (NGAL), is a beta barrel secreted protein that binds siderophore-complexed ferric iron with high affinity. SLC22A17 is a LCN2 transporter expressed in a various cell types, including cancer cells. To investigate the functional consequences of LCN2/SLC22A17 expression, three mouse models of LM generated through iterative in vivo selection were used (Table 2). Unselected parental cells (Par) are nonspecifically metastatic; the LM subpopulation of cells (LeptoM) readily enter into and grow within the leptomeningeal space. The mouse lung adenocarcinoma (LLC-Par, LLC-LeptoM), human lung adenocarcinoma (PC9-Par, PC9-LeptoM) and human breast adenocarcinoma (MDA231-Par, MDA231-LeptoM) models all share key features of human LM.

TABLE 2
Mouse Model Characteristics

| | | | | | Time to | Survival | CSF |
|---|---|---|---|---|---|---|---|
| | | Host | Cancer Cell | | LM | Post LM | Protein |
| Model ID | Species | Mouse | Tissue Origin | Subtype | (Days) | (Days) | (mg/dL) |
| PC9-Par | Human | NCI nu/nu | Lung | EGFR ΔE746-750 | — | — | 9.0 ± 1.8 |
| PC9-LeptoM | Human | NCI nu/nu | Lung | EGFR ΔE746-750 | 7 | 30.5 ± 9.5 | 42.7 ± 4.4 |
| LLC-Par | Mouse | C57/B16 | Lung | Adenocarcinoma | — | — | 18.7 ± 5.5 |
| LLC-LeptoM | Mouse | C57/B16 | Lung | Adenocarcinoma | 14 | 17.5 ± 3.5 | 62.5 ± 3.8 |
| MDA231-Par | Human | NCI nu/nu | Breast | Basal/Triple negative | — | — | 41.0 ± 3.6 |
| MDA231-LeptoM | Human | NCI nu/nu | Breast | Basal/Triple negative | 14 | 28 ± 7 | 61.2 ± 3.5 |

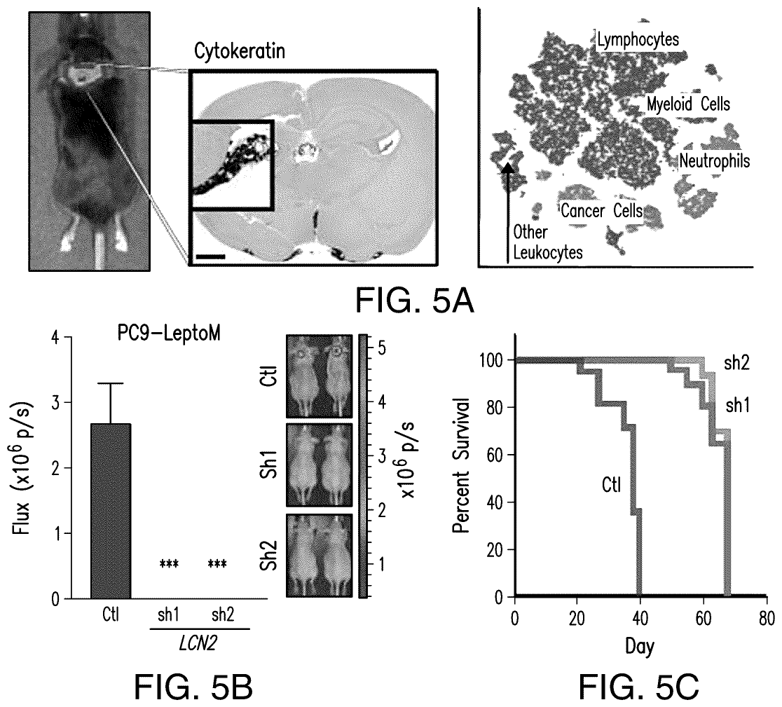

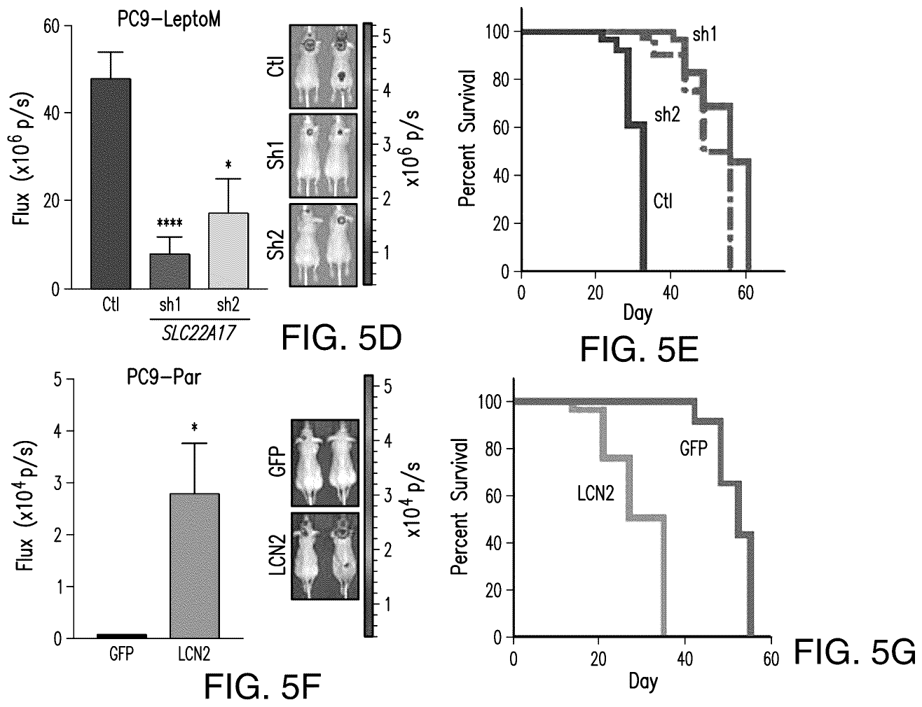

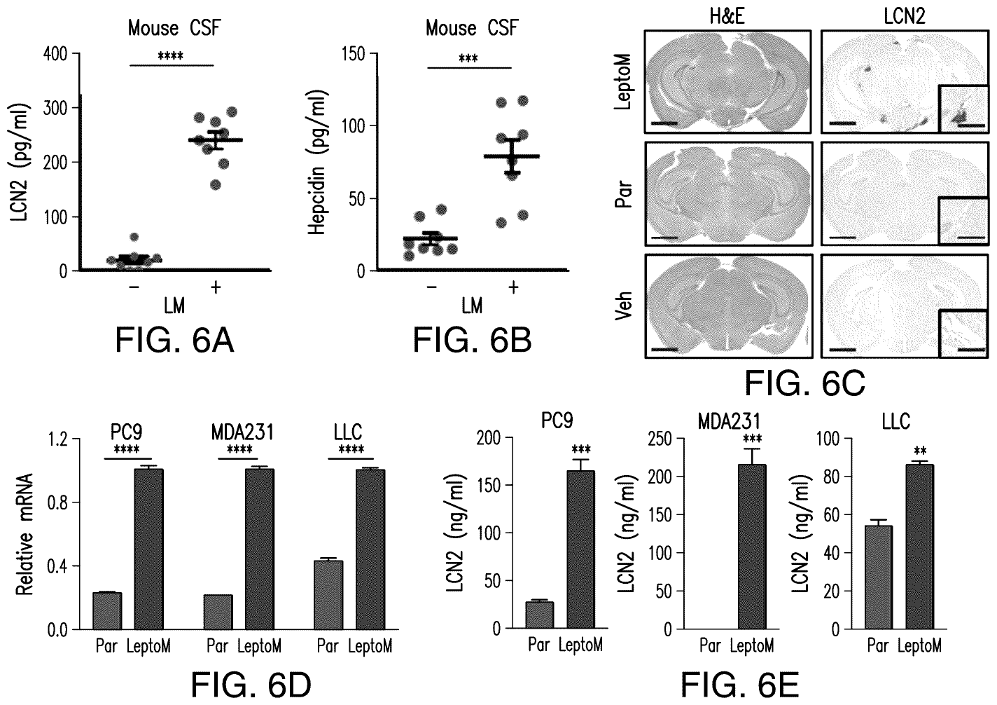

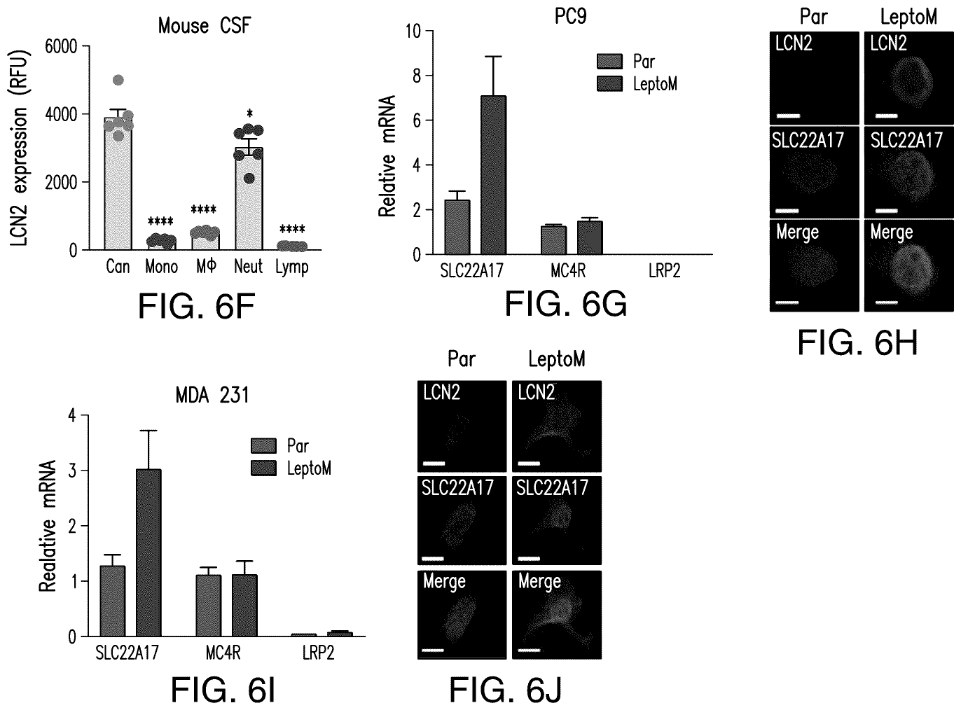

Flow cytometry of CSF from the immunocompetent LLC-LeptoM mouse model revealed infiltrating cancer cells accompanied by lymphocytes, macrophages and neutrophils (FIG. 5A). Concentrations of hepcidin and LCN2 were elevated in the CSF of mice with LM (FIGS. 6, A and B). LCN2 staining by immunohistochemistry (IHC) was specific to cancer cells within the leptomeningeal space (FIG. 6C). Higher levels of LCN2 mRNA and protein were found in LeptoM cells than in their parental (Par) counterparts (FIGS. 3D and E). While previous studies have documented LCN2 expression in activated macrophages within other anatomic sites (e.g., mammary fat pad), LCN2 was found to be expressed by cancer cells and not macrophages within the leptomeningeal space (FIG. 6F). LCN2 binds productively to three known receptors (SLC22A17, LRP2 and MC4R). In both humans and mouse models, SLC22A17 constituted the major LCN2 receptor (FIGS. 1F and H; FIG. 6G to J).

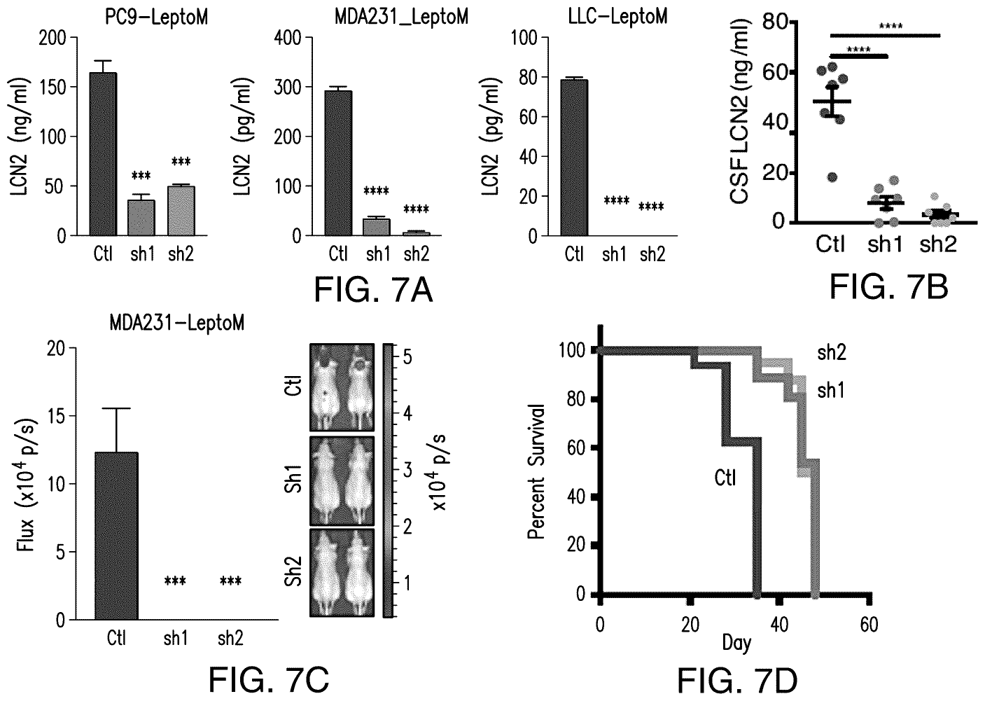

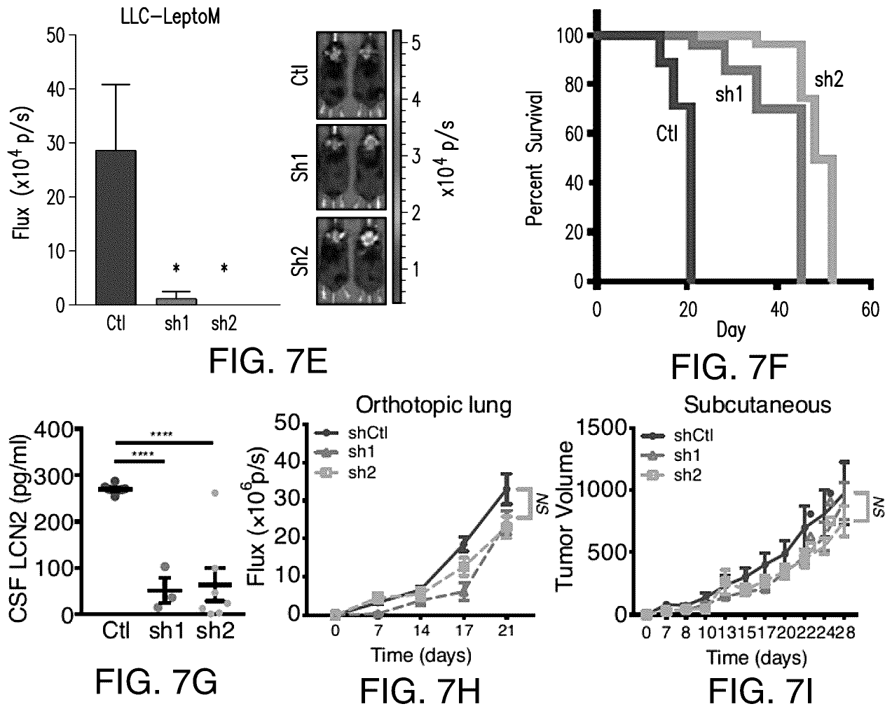

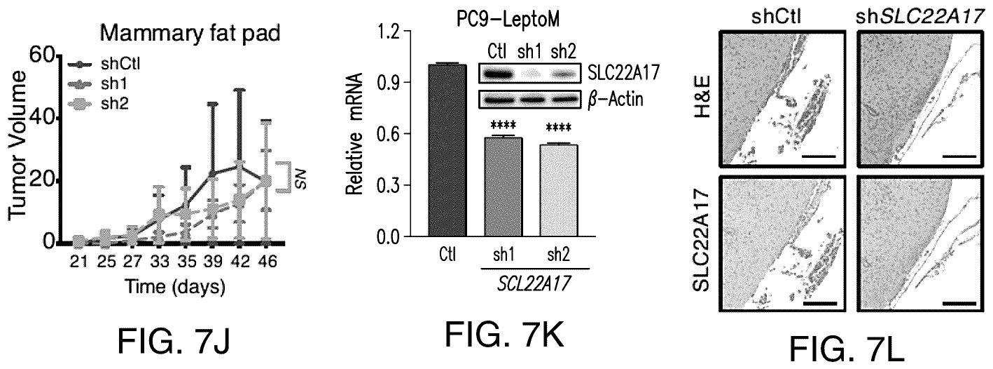

LCN2 promotes cancer cell growth within the leptomeningeal space in mice. The functional relevance of LCN2/SLC22A17 in the CSF was investigated next. It was found that shRNA-mediated loss of LCN2 expression in LeptoM cells inhibited their growth within the leptomeninges in all three mouse models and conferred a survival benefit to the animals (FIG. 5B-C, FIG. 7A to G). In contrast, LCN2 knockdown did not alter growth of LeptoM cells within iron-replete anatomic sites in vivo (FIG. 7H to J). Knockdown of the LCN2 receptor, SLC22A17, phenocopied these results (FIG. 5D-E, and FIG. 7K-L). Conversely, overexpression of LCN2 in parental (Par) cells with no propensity for growth in the leptomeninges promoted growth of these cells in the leptomeningeal space and hastened the death of these animals (FIG. 5F-G, FIG. 8). Together, these results support a mechanistic role for the LCN2/SLC22A17 axis in leptomeningeal cancer cell growth.

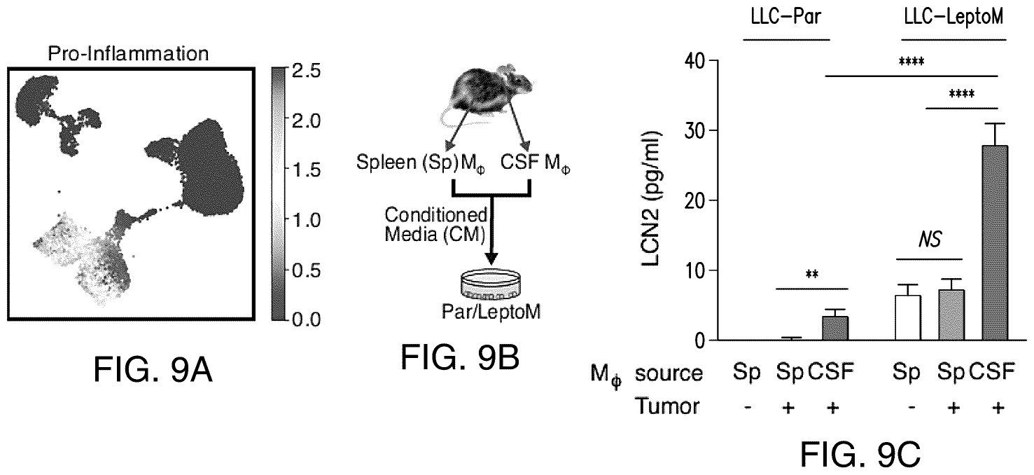

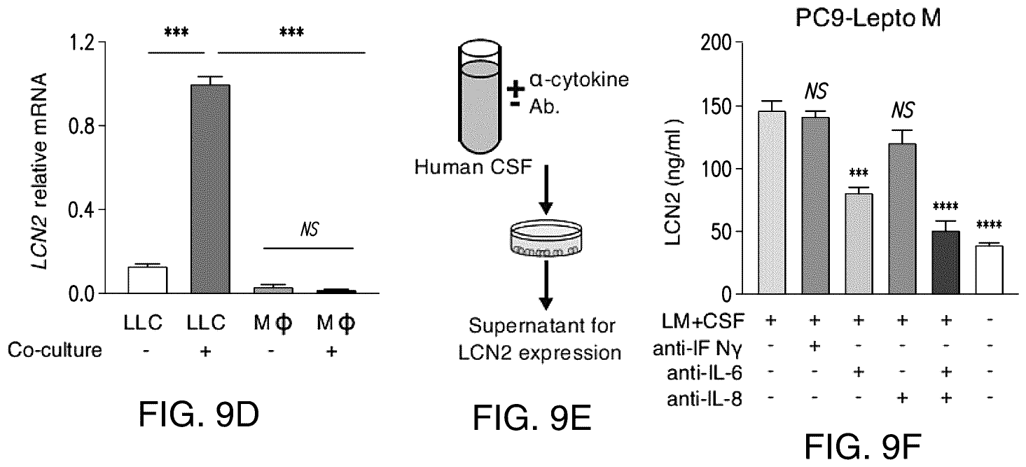

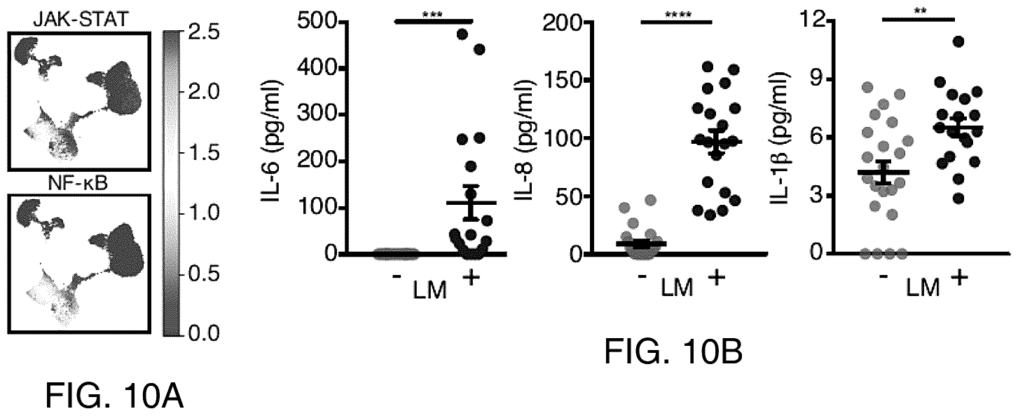

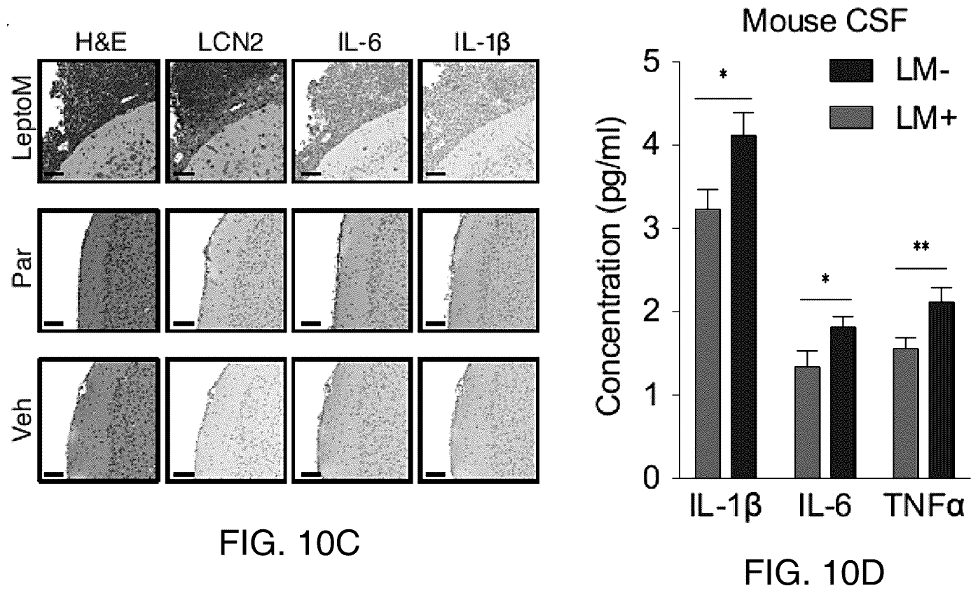

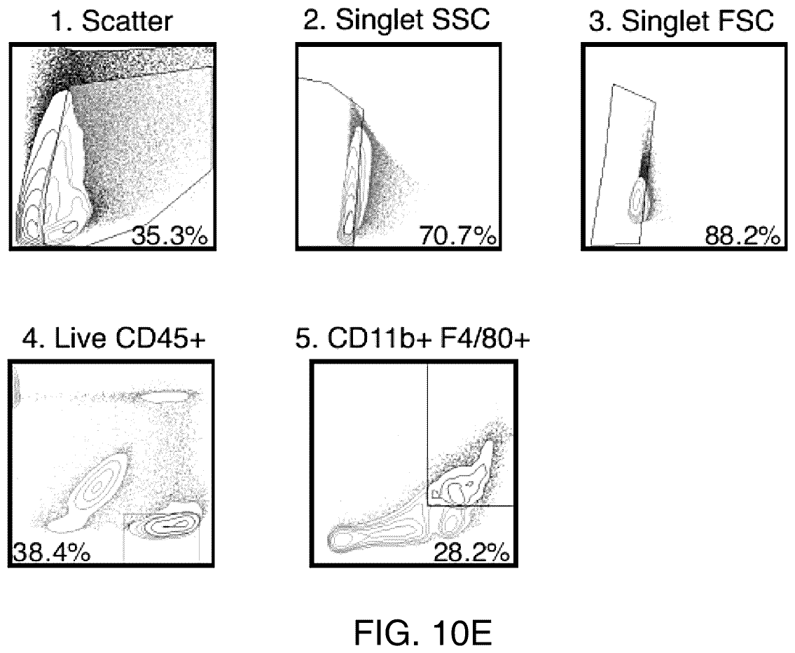

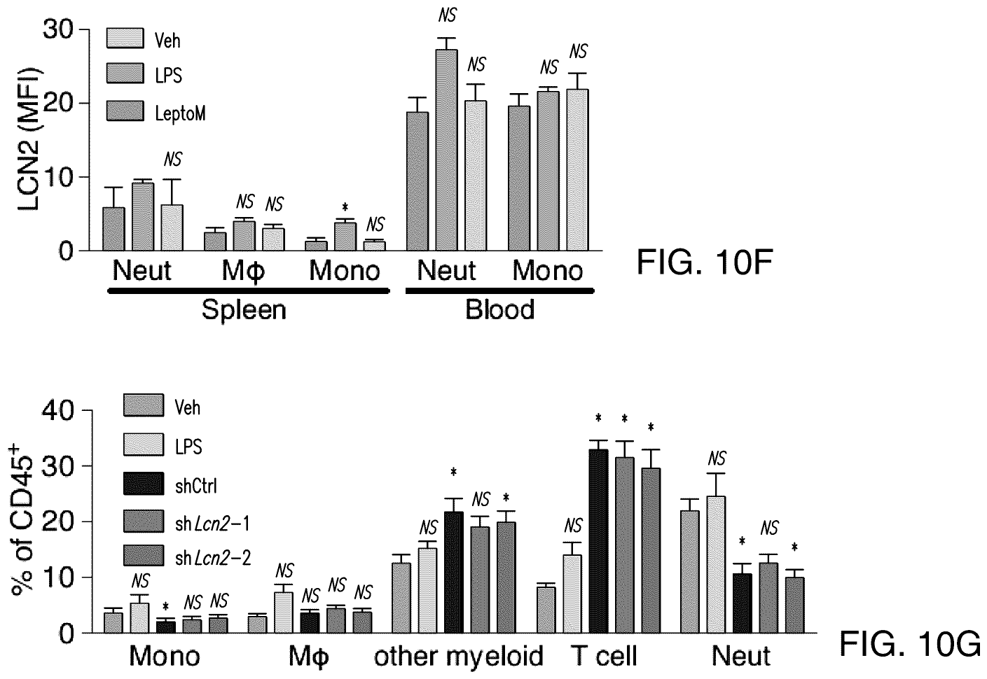

Inflammatory cytokines induce LCN2 expression in cancer cells. The mechanism leading to LCN2 expression in cancer cells within the LM was next explored. Downstream of both STAT and NF-κB transcriptional promoters, expression of LCN2 may be induced by a variety of inflammatory stimuli (15, 16). To assess the inflammatory state of cells during LM, the scRNA-Seq dataset was queried and found high expression levels of transcripts downstream of JAK-STAT and NFKB promoters in the macrophage population (FIG. 9A, FIG. 10A). Consistent with this, inflammatory cytokine (IL-6, IL-8 and IL-1β) concentrations were significantly higher in the CSF of cancer patients with LM versus control patients without LM (FIG. 10B). This was also observed in mouse models (FIG. 10C-D). It was hypothesized that macrophage-generated cytokines stimulated cancer cell LCN2 expression in the CSF.

To test this hypothesis, LeptoM cancer cells were co-cultured with supernatant collected from macrophages freshly isolated from either the CSF or spleen of mice harboring LLC-LeptoM or LLC-Par tumor cells (FIG. 9B and FIG. 10E). It was found that macrophages from the CSF of mice harboring LM strongly induced LCN2 expression in LeptoM cancer cells (FIG. 9C). Co-culture of these CSF macrophages with LeptoM cancer cells induced expression of LCN2 in cancer cells but not in macrophages (FIG. 9D). Outside the leptomeninges, LCN2 expression by macrophage/monocytes and neutrophils is typically induced by inflammation. In these models, although LCN2 expression was increased in extracranial splenic monocytes and neutrophils after lipopolysaccharide (LPS) treatment, LCN2 expression remained unchanged in these cells in the CSF (FIG. 4G, FIG. 6F, FIG. 10F). Moreover, while LCN2 may induce influx of neutrophils to extracranial sites, LCN2-dependent changes in CSF leukocyte composition were not appreciated (FIG. 10G), underlining the unique physiology of the leptomeninges.

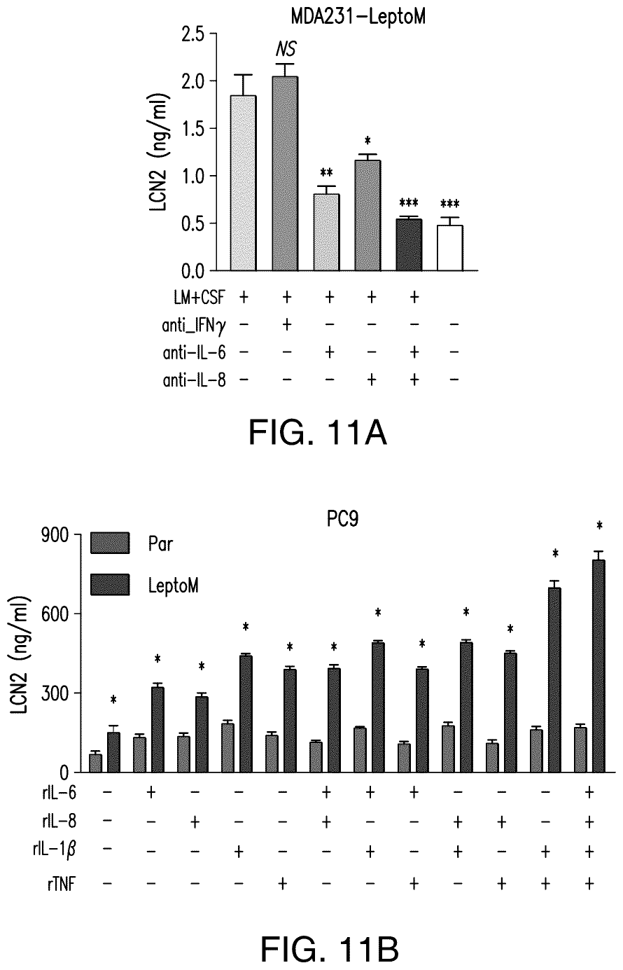

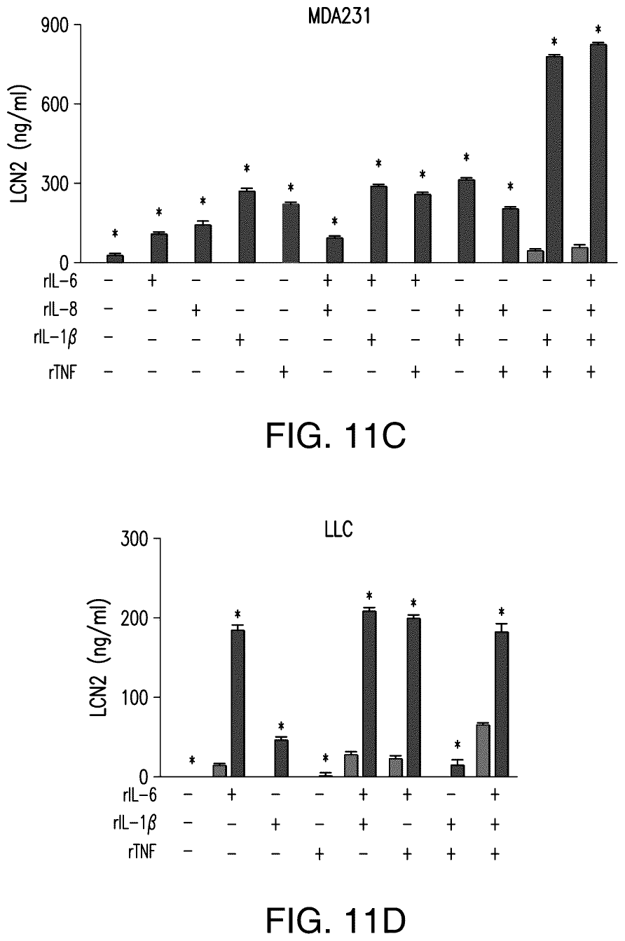

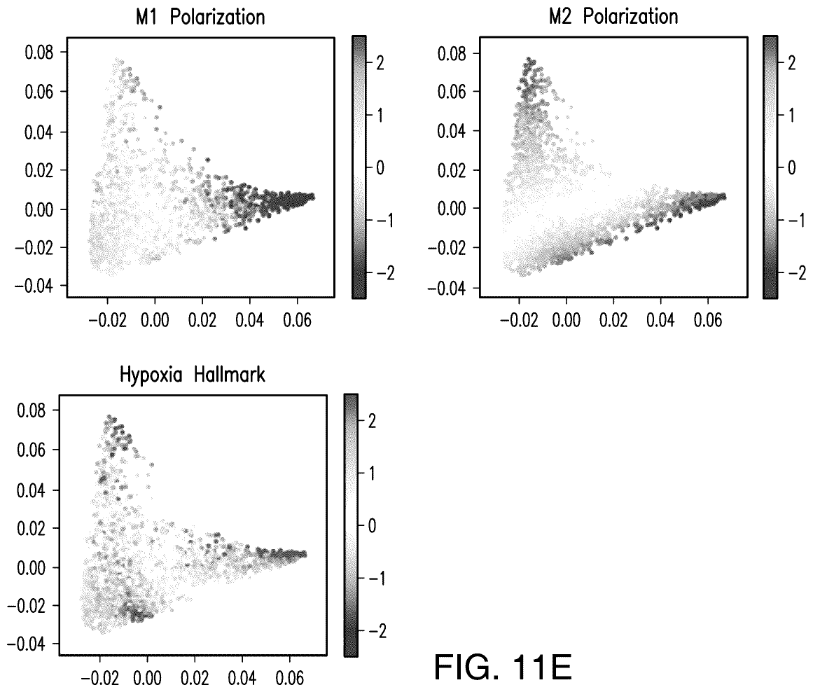

To identify the relevant cytokines in human disease, select inflammatory cytokines were immune-depleted from LM-positive human CSF (FIG. 9E). Whereas whole CSF from LM patients strongly induced LCN2 expression in cancer cells, immune-depletion of IL-6 and/or IL-8 or inhibited this biological effect in both PC9 and MDA231 model systems (FIG. 9F and FIG. 11A). Conversely, addition of recombinant IL-6, IL-8 or IL-1β to artificial CSF induced expression of LCN2 from PC9, MDA231 or LLC (FIG. 11B to D) LeptoM cells. Together, these data indicate that macrophage-generated cytokines induce expression of LCN2 in cancer cells within the LM.

To gain more insight into the properties of these pro-inflammatory leptomeningeal macrophages, the transcriptome of the macrophage/monocyte population in the scRNA-Seq dataset was examined. Consistent with the known transcriptional heterogeneity of these cells, non-reciprocal expression of both M1 (proinflammatory) and M2 (anti-inflammatory) polarization transcriptional gradients was observed (FIG. 11E). Unexpectedly, this analysis revealed the hypoxia transcriptional signature as a major feature of this macrophage population.

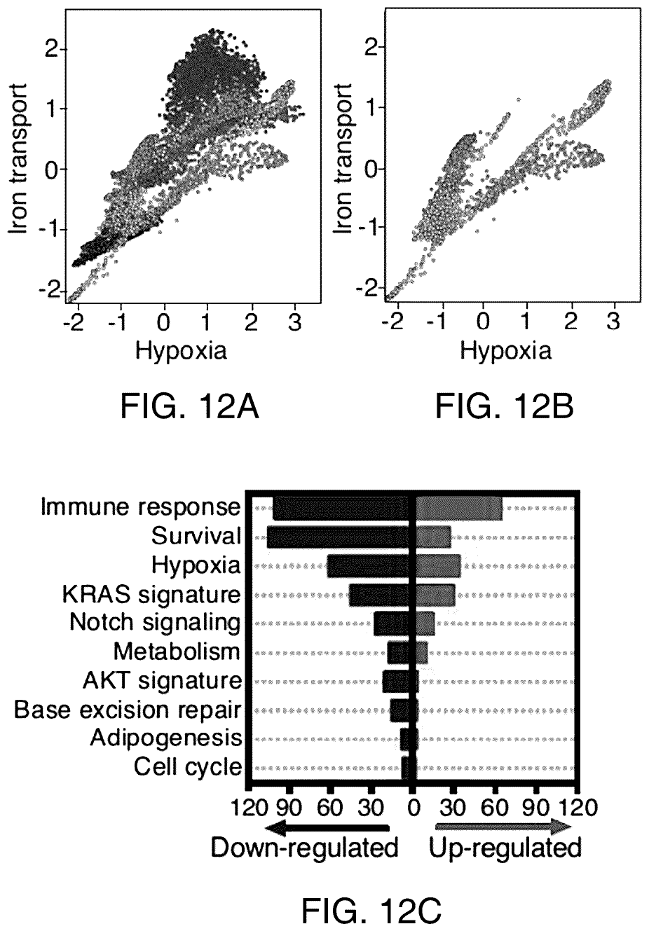

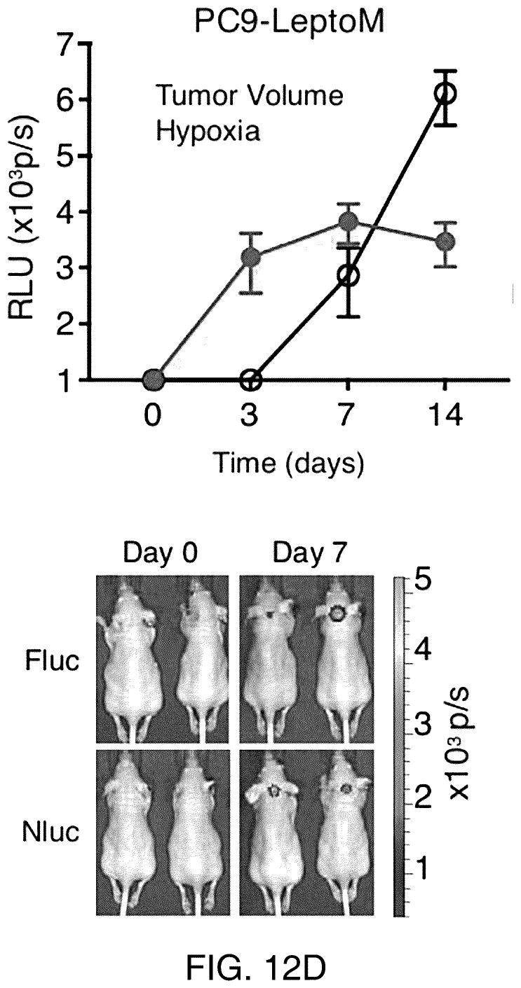

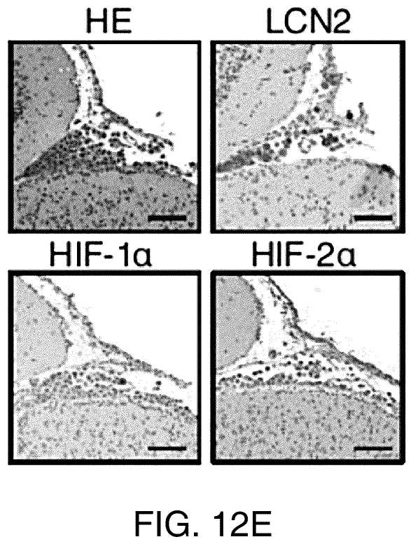

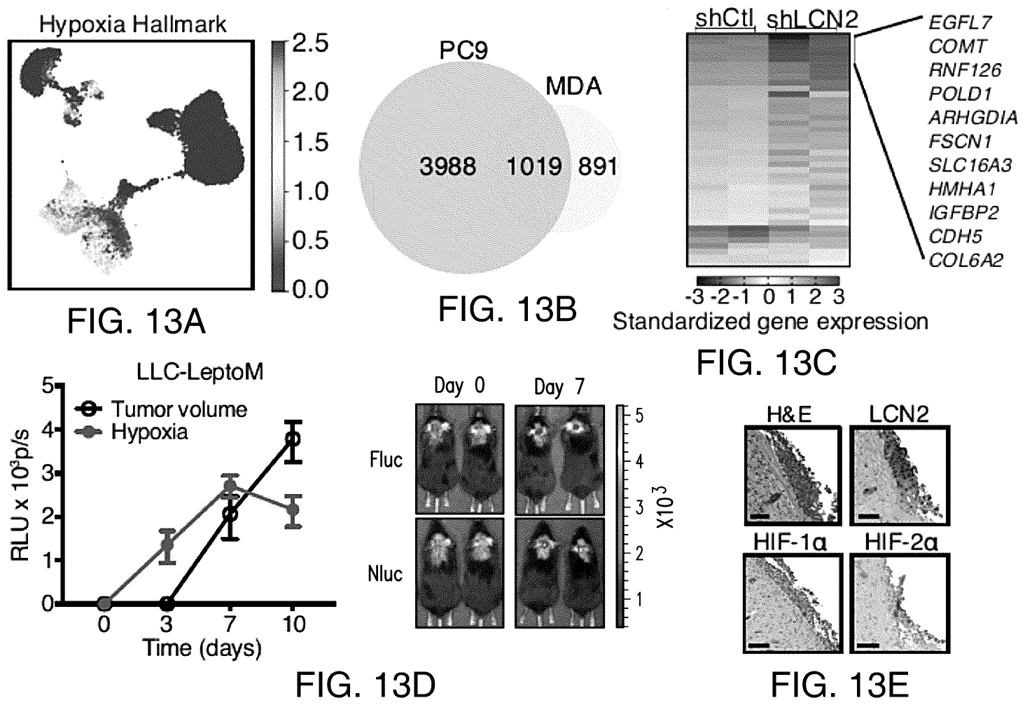

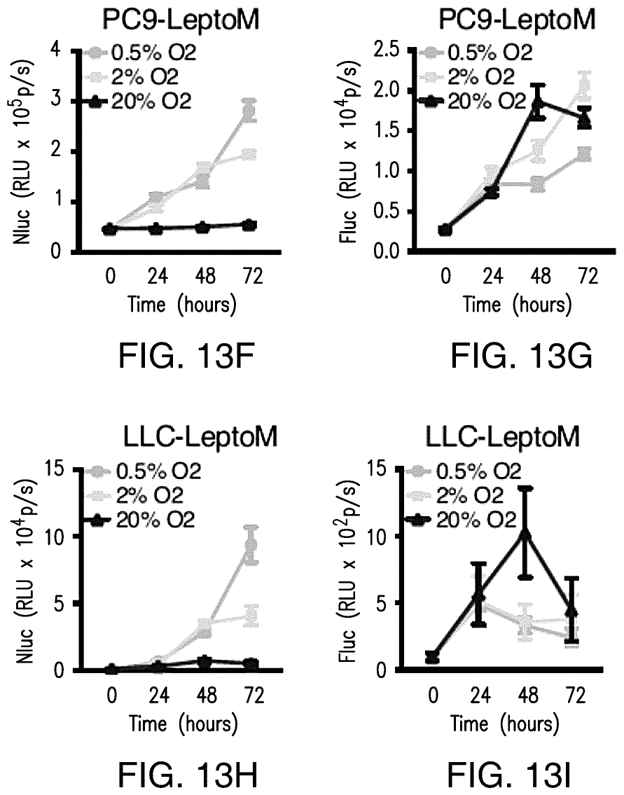

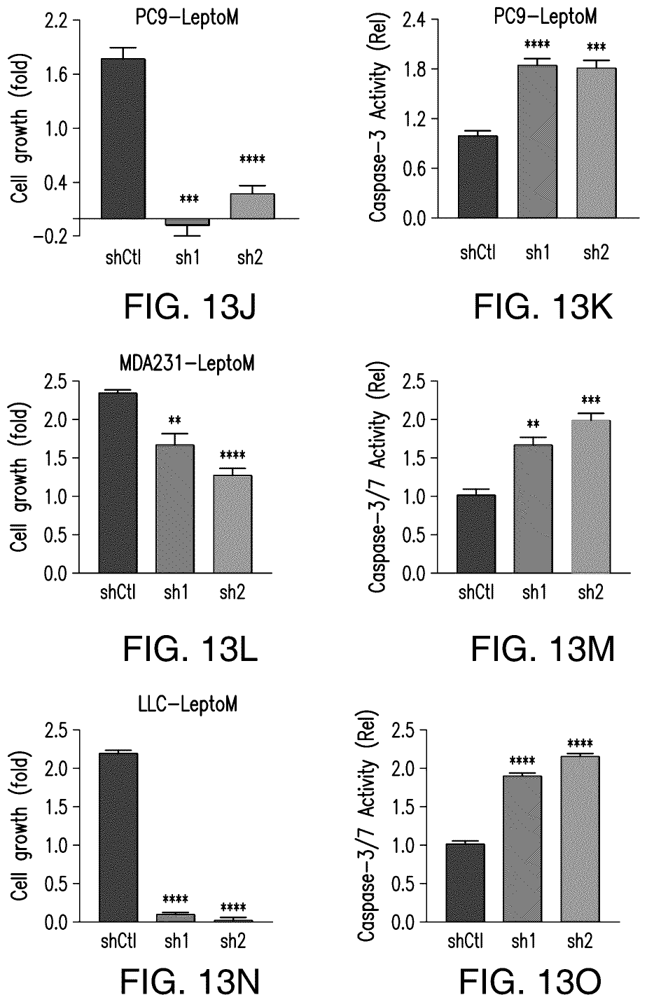

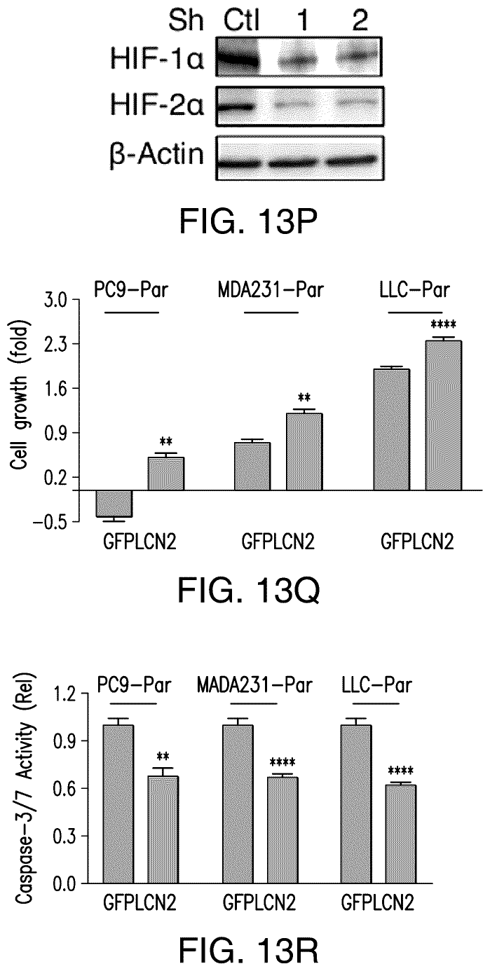

LCN2 supports cancer cell growth in the hypoxic leptomeninges in mice. Hypoxia signaling is intimately linked to iron homeostasis. It was found that freshly isolated CSF cancer cells and immune cells from human LM samples showed evidence of hypoxia-induced transcriptional changes as well as a correlation between hypoxia and iron metabolic signatures (FIGS. 12A and B and FIG. 13A). In mouse models, bulk RNA-Seq of LeptoM derivative cell lines with and without LCN2 knockdown revealed LCN2-dependent expression of the hypoxia transcriptional signature, suggesting a role for LCN2 in hypoxia (FIG. 12C and FIG. 13B-C).

The biological relevance of hypoxia signaling in LM remains undetermined. In the absence of disease, the CSF is hypoxic with ppO₂ ranging from 65-130+/−49 mmHg. However, in inflammatory injury or an impaired blood-CSF-barrier CSF oxygenation is improved. To study hypoxia in these LM mouse models a dual-luciferase reporter system consisting of constitutive firefly luciferase and nano-luciferase downstream of hypoxia response elements (HREs) was generated. The PC9-LeptoM and LLC-LeptoM models demonstrated NLuc activation (an indication of hypoxia) upon inoculation into the CSF and this activation remained stable over the disease course (FIG. 12D). Despite this hypoxia, the LeptoM cells continued to grow, and expressed hypoxia-inducible factors HIF1A and HIF2A (FIG. 4D-E and FIG. 13D-E). In vitro, LeptoM derivatives demonstrated robust growth under hypoxic conditions in an LCN2-dependent manner: Knockdown of LCN2 expression with shRNA inhibited LeptoM growth and promoted apoptosis in hypoxia (FIG. 13F to P), overexpression of LCN2 provided resistance to hypoxic stress (FIG. 13Q-R). From these experiments, it was concluded that cancer cell LCN2 expression supports cancer cell growth within hypoxic CSF in LM.

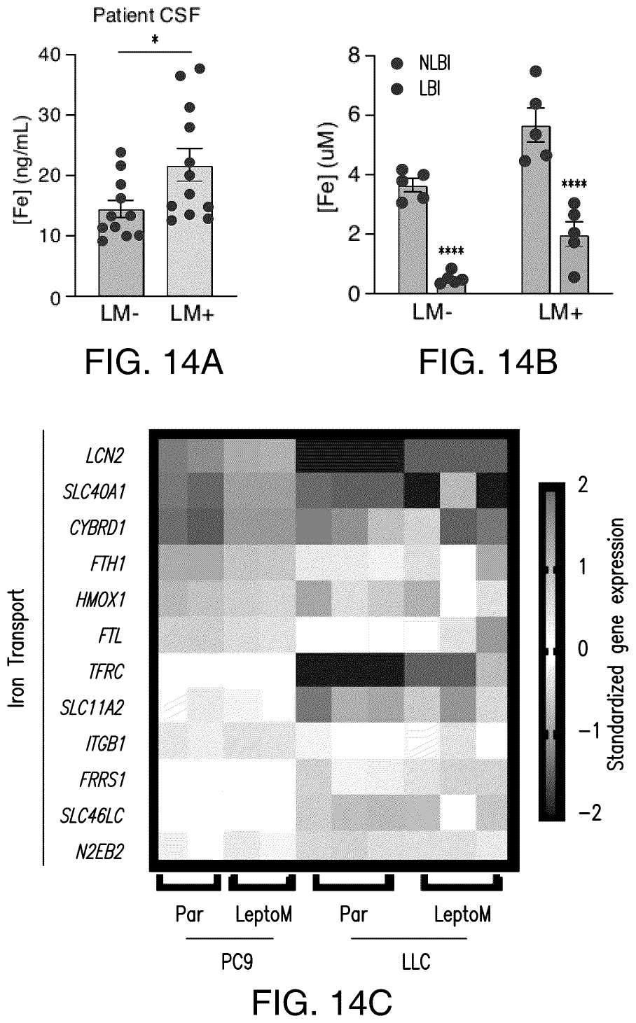

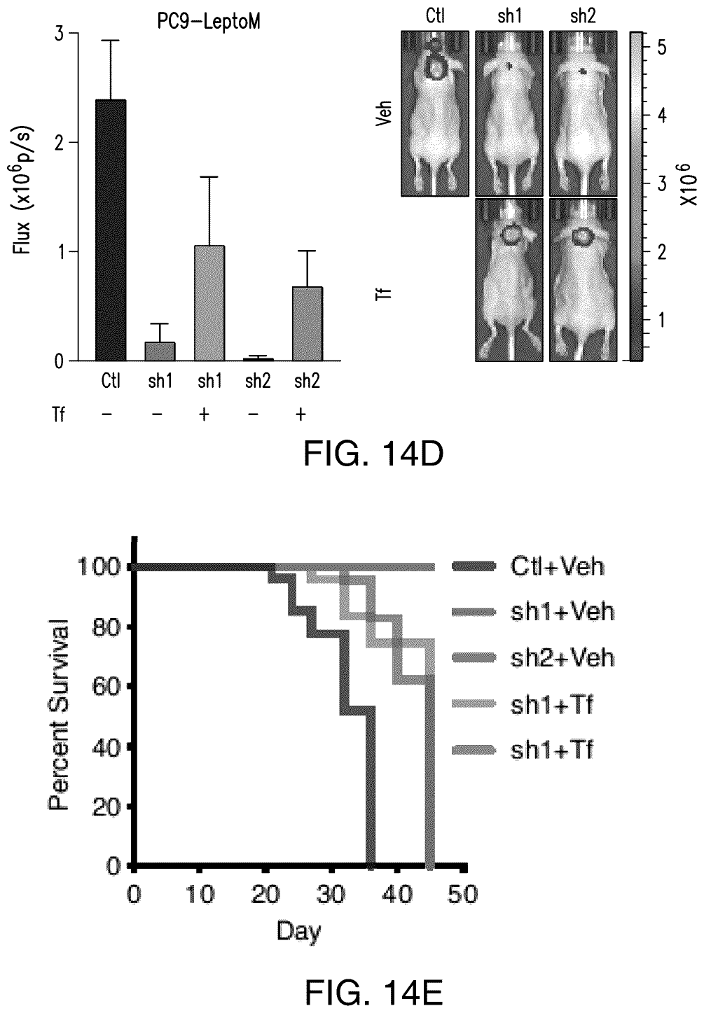

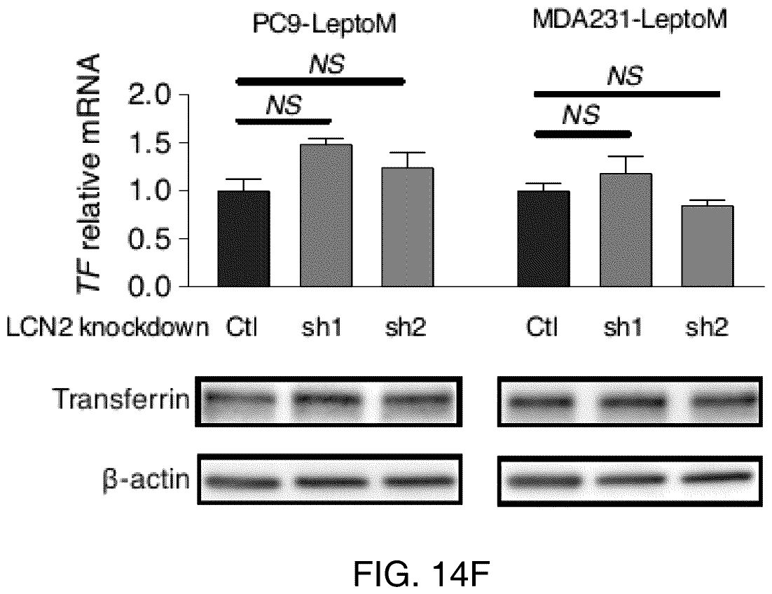

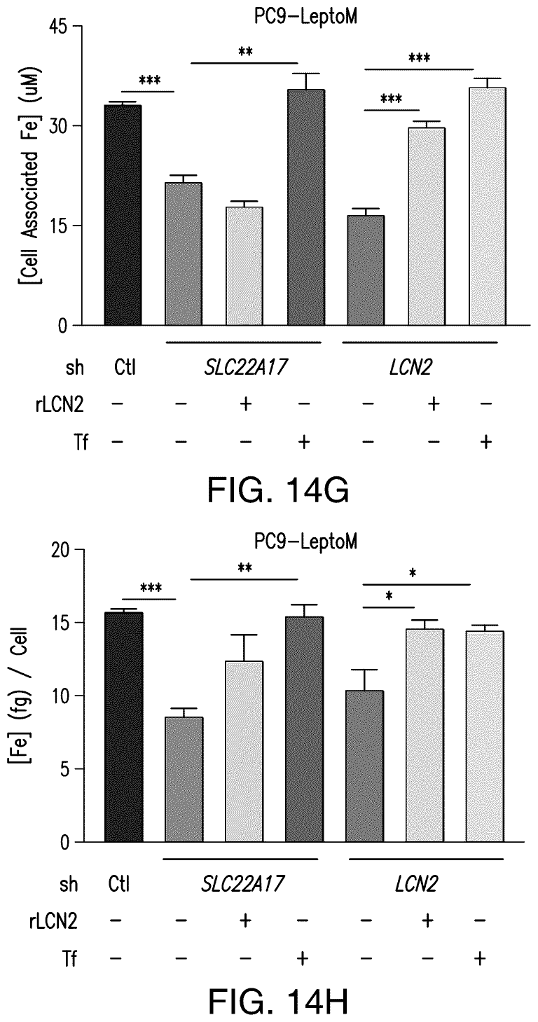

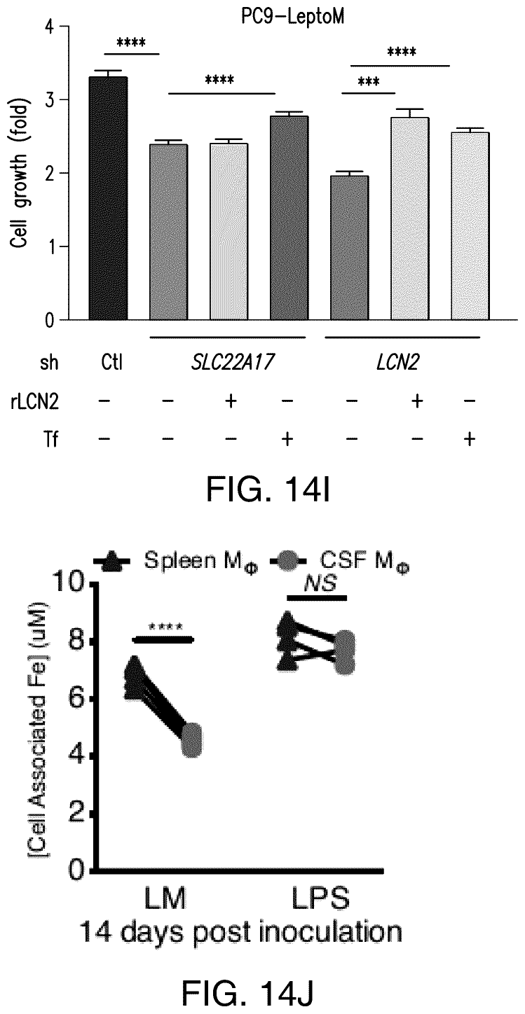

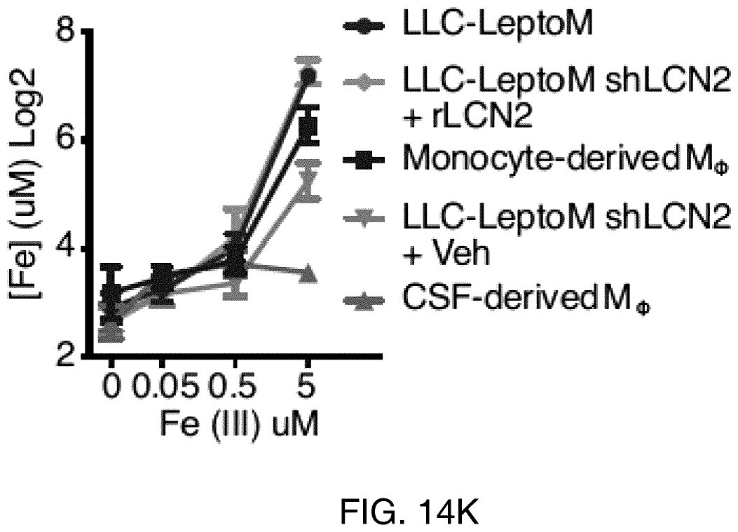

Cancer cells employ LCN2 to collect sparse extracellular iron in the CSF. In the absence of disease, CSF contains minimal extracellular iron. To investigate the levels of this micronutrient in LM, total iron levels were assayed by mass spectroscopy in the CSF of cancer patients. It was found that total iron concentration and the proportion of iron bound to LCN2 was increased in the CSF of patients with LM compared with patients without LM, (FIGS. 14A and B). In addition, patient CSF LCN2 levels correlated with iron concentration (Table 3). Reflecting the importance of inflammatory signaling, CSF iron levels also correlated with IL-6 and hepcidin.

Turning to these mouse models, it was found that LCN2 gene expression was upregulated in LeptoM cells when compared with Par cells (FIG. 6D and FIG. 14C). In vivo, LCN2 knockdown by shRNA was partially rescued through addition of iron-loaded transferrin (FIG. 5A-B, and FIG. 14D-F), suggesting that iron transport plays a key role in LCN2-dependent cancer cell growth in the CSF. To investigate this further, iron uptake was examined in these LeptoM cells in vitro. Inhibition of either SLC22A17 or LCN2 expression in LeptoM cells by shRNA in vitro inhibited iron accumulation and cell growth (FIG. 14G-I). Intracellular iron accumulation and cell growth were rescued by addition of exogenous transferrin (FIG. 14G-I).

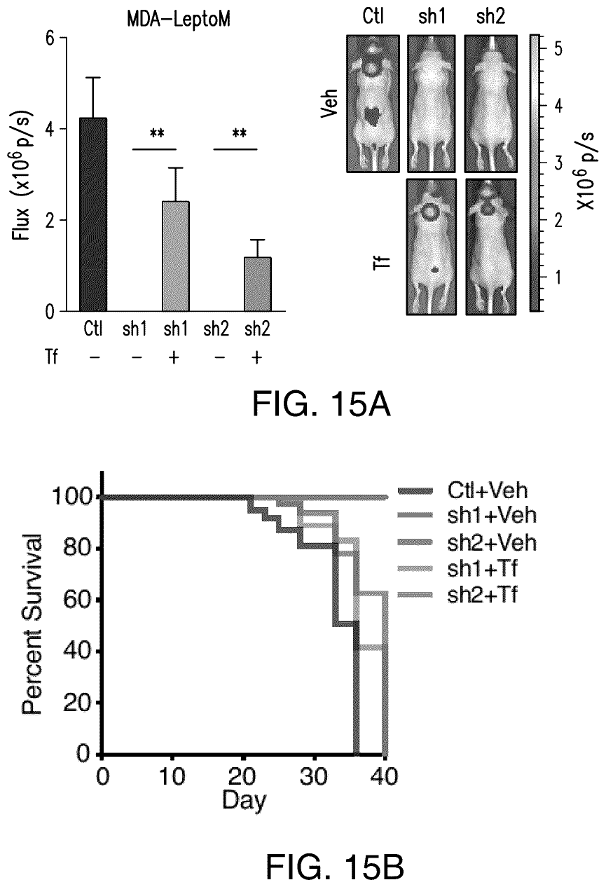

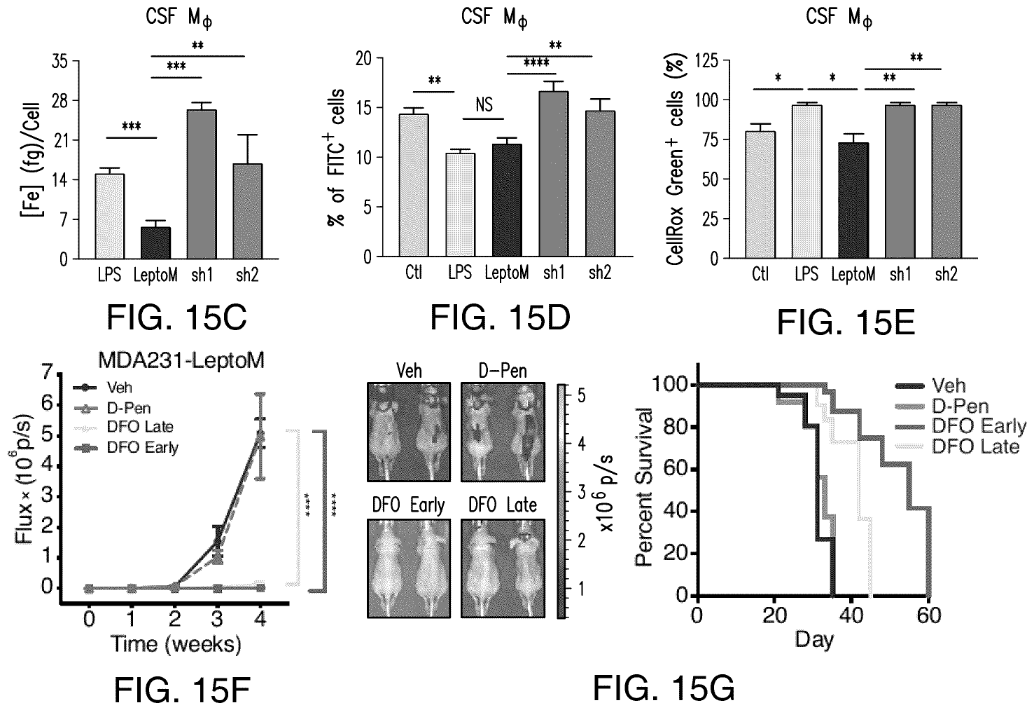

To generate reactive oxygen species, activated macrophages have a heightened need for iron. It was hypothesized that macrophages within the CSF, particularly in LM, have lower iron stores than macrophages circulating in the blood. To address this, the syngeneic LLC-LeptoM mouse model was studied (Table 2). Macrophages were collected from either the CSF or the spleen in the setting of LM or after challenge with lipopolysaccharide (LPS) (FIG. 15F). It was found that the intracellular iron content of CSF macrophages declined significantly in the setting of LM when compared with LPS treatment (FIG. 15C and FIG. 14J). It was also found that shRNA-mediated knockdown of cancer cell LCN2 expression increased macrophage iron content (FIG. 15C and FIG. 14K). Finally, it was found that the impairment of iron uptake in the CSF has functional consequences for these macrophages: both respiratory burst and phagocytosis were impaired in the setting of LM in a LCN2-dependent fashion (FIG. 15D-E). Together, these observations are consistent with a model in which inflammatory signals promote cancer cell LCN2 production. This, in turn, allows cancer cells to acquire the iron that is present in limiting amounts in CSF, which not only supports their own growth but inhibits iron uptake and iron-dependent functional activities of macrophages.

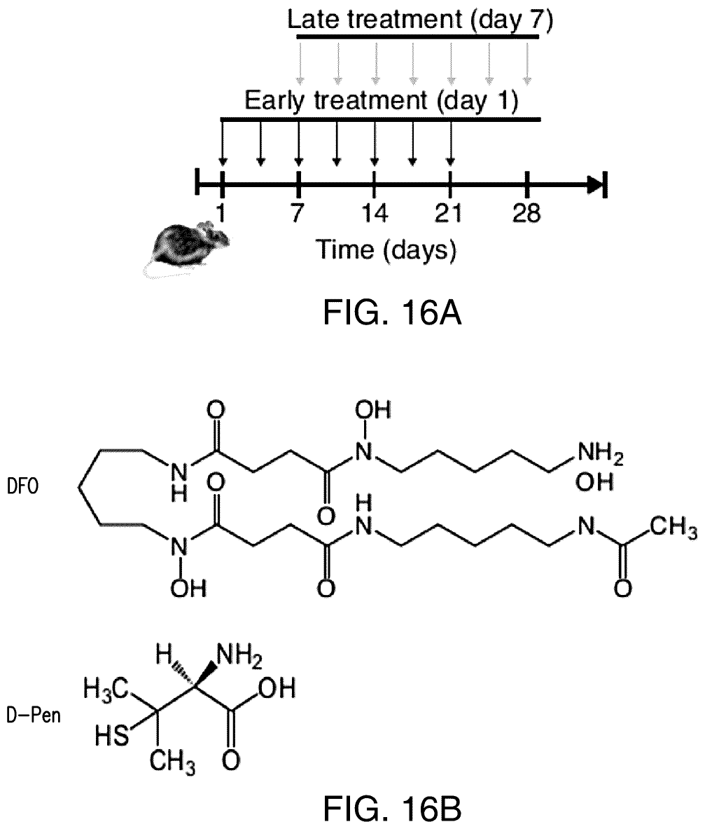

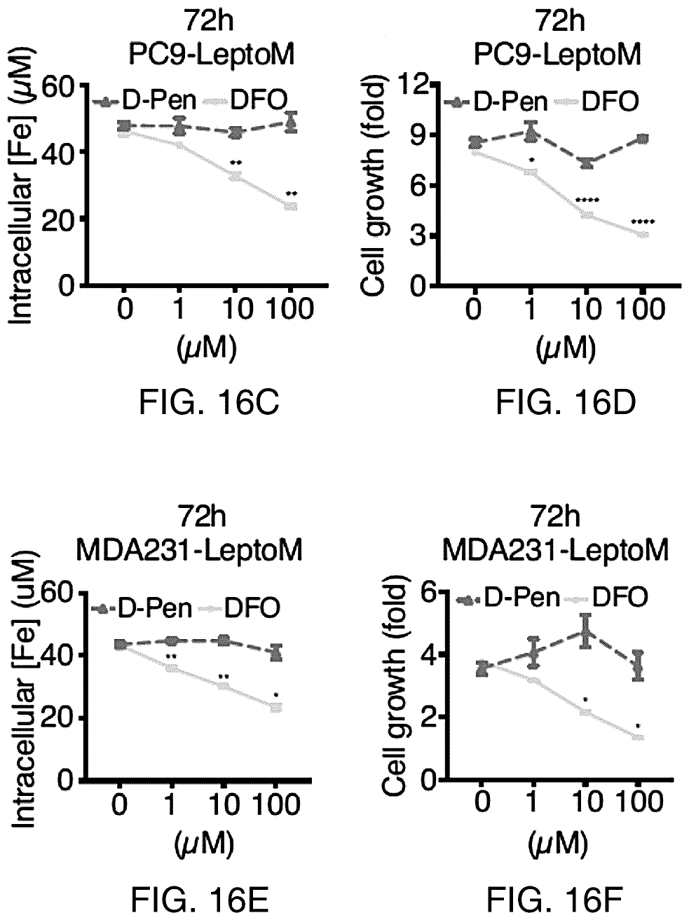

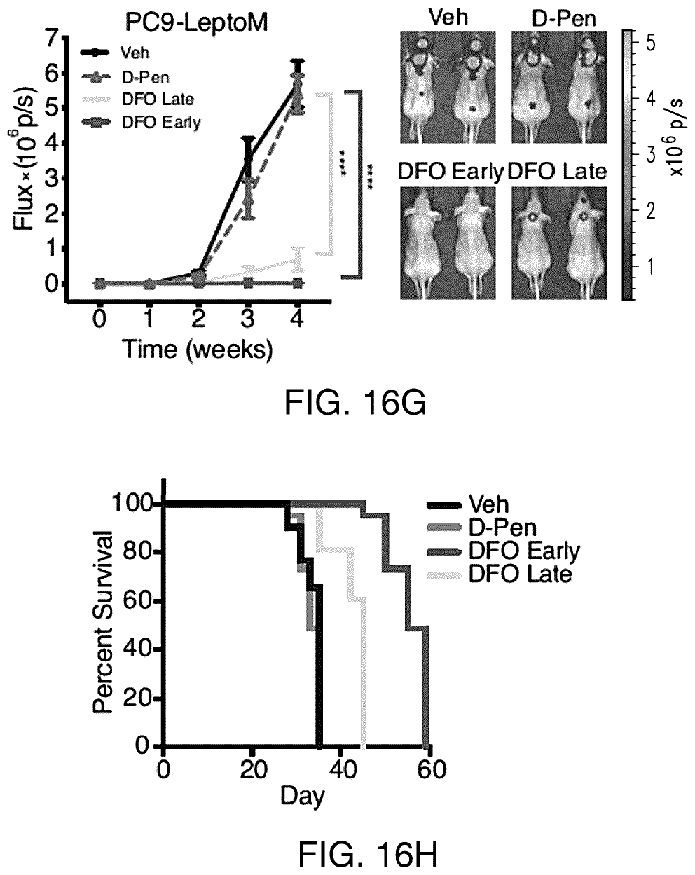

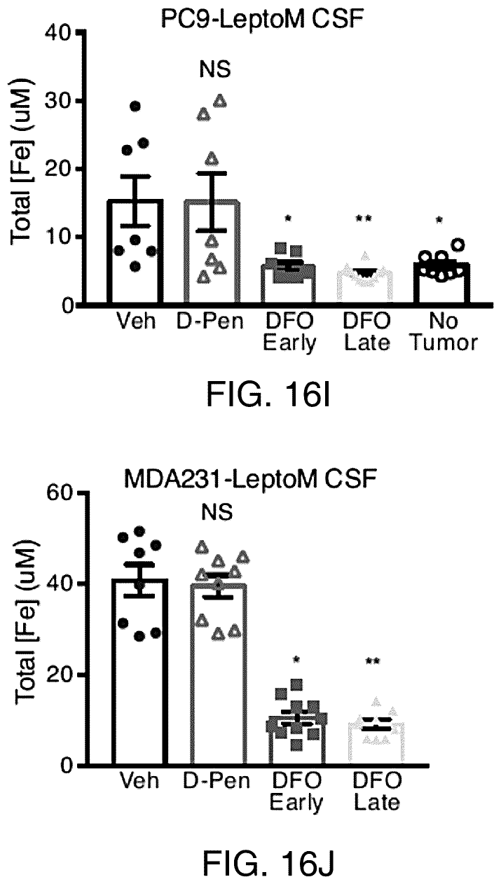

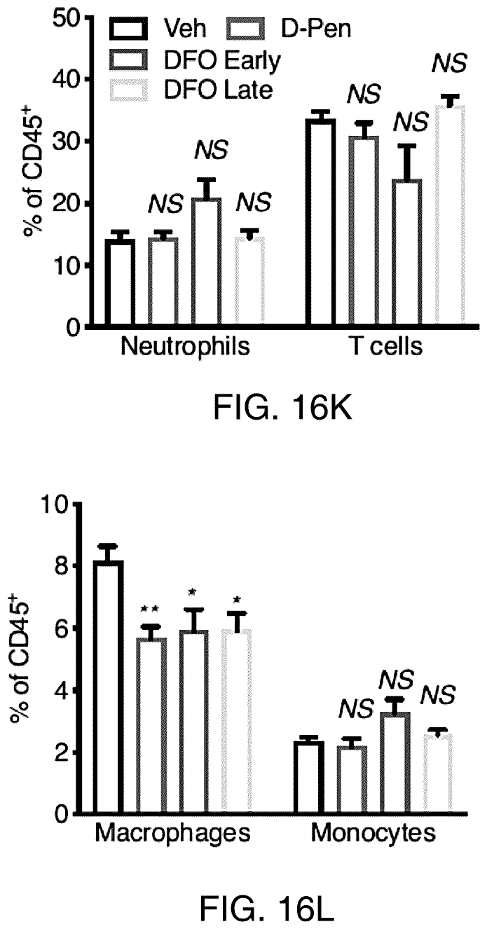

Analysis of iron chelation therapy for LM in mice. Because iron is limiting in the CSF, it was reasoned that iron chelation might impair cancer cell growth in the CSF and tested this hypothesis in these mouse models (Table 2). Recipient mice was inoculated with either MDA231-LeptoM or PC9-LeptoM cells and treated them intracisternally with vehicle, the iron chelator deferoxamine (DFO), or the copper chelator D-penicillamine (D-Pen) on day 0 or day 7 after engraftment and every 3 days thereafter (FIG. 16A-B). DFO treatment substantially suppressed iron levels within the LeptoM cells as well as their growth (FIG. 5F and FIG. 16C to F). Importantly, DFO treatment conferred a survival benefit to the MDA231 and PC9 mouse models compared with vehicle control (FIG. 5G, FIG. 16G-H and Table 4). As expected for this chelator treatment, it was found that iron concentration in the CSF from DFO treated mice was decreased at day 28 compared to that in control mice (FIG. 16I-J). The number of CSF macrophages was slightly reduced by the D-Pen or DFO treatment, whereas the numbers of neutrophils, T cells and monocytes were not affected (FIG. 16K-L).

Discussion:

Circulating immune cells routinely encounter nutritionally sparse environments as they migrate from the circulation into the tissues. Indeed, immune cells employ a discrete set of transcriptional programs to make use of limited resources, including iron and oxygen. In the case of inflammatory macrophages, interferon-γ signaling promotes generation of nitric oxide, impairing oxidative phosphorylation (OXPHOS) and allowing for generation of reactive oxygen species (ROS).

Cancer cells cope with challenging environmental constraints differently from immune cells. The genetic heterogeneity of cancer cells provides these cells with a selective advantage over other cell types. To study cellular competition for sparse nutrients at human scale, single-cell RNA-Seq was applied to patient-derived samples of LM, a lethal complication of cancer. In doing so, it was uncovered a specific example of how the dynamic transcriptional heterogeneity of cancer cells can confer a functional selective advantage. It was found that cancer cells make use of LCN2/SLC22A17, a high-affinity iron collection system that enables them to effectively outcompete other cells in the leptomeninges for sparse environmental iron. The other major iron-utilizing cell in the CSF, the macrophage, is rendered iron-deficient by this process, resulting in impaired respiratory burst and phagocytosis.

The leptomeninges pose unique constraints on both the infiltrating immune system and cancer cells; it was found that inter-cellular signaling is substantially altered within the leptomeningeal space. Investigators studying other experimental systems focused on extracranial sites have observed macrophage LCN2 generation, promoting cancer cell migration and invasion. In LM, CSF macrophages do not produce LCN2. Rather, the inflammatory microenvironment promotes the generation of LCN2 in cancer cells. As obligate partners for cancer cell migration, invasion and metastasis, tumor-associated macrophages are well known to alter the behavior of cancer cells. The evolutionary dynamics that were discovered between malignant and non-malignant cells within the leptomeninges reveal both the robust nature of cancer's transcriptional plasticity and highlight microenvironmental vulnerabilities ripe for therapeutic exploitation.

### Example 2: Phase VII Dose Escalation Clinical Study

This Example discloses details of the Phase I/II dose escalation trial to assess safety and bioactivity of intrathecal desferoxamine in the treatment of leptomeningeal metastases from recurrent non-small cell lung cancer.

Protocol Summary and/or Schema:

This study, A Phase I/II Dose Escalation Trial to Assess Safety and Bioactivity of Intrathecal Desferoxamine in the Treatment of Leptomeningeal Metastases from Recurrent Non-Small Cell Lung Cancer, aims to evaluate the use of iron chelation therapy in patients with recurrent leptomeningeal metastases. The use of desferoxamine been previously demonstrated to be safe and effective when given systemically (IV/IM/PO) for various medical conditions, but only recently has demonstrated clinical activity in the prevention of leptomeningeal metastasis growth in pre-clinical mouse models when given intrathecally. The purpose of this study is to determine the optimal dosing, toxicity profile, and biologic activity of intrathecal desferoxamine (IT-DFO) in the treatment of recurrent previously-irradiated leptomeningeal metastases from non-small cell lung cancer (NSCLC).

Patients must be at least 18 years of age or older, Karnofsky performance status (KPS)≥50, previously had received CNS-directed radiation for treatment of their NSCLC leptomeningeal metastases at least 60 days from the time of study entry, and have confirmed leptomeningeal metastasis progression following radiation therapy based on cytologic, CSF circulating tumor cell, and/or radiographic analysis. Patients will require Ommaya reservoir placement for drug administration and serial CSF sampling. Patients can be continued on CNS-penetrant systemic tumor-directed therapy, provided that the patient has already developed CNS progression on this agent or the systemic agent has no evidence of efficacy in the CNS.

Phase 1: The phase I arm of this trial will include a 3+3 dose escalation scheme to determine the maximum tolerated dose (MTD) of IT-DFO. Patients will be treated with a 3+3 design for cohorts 1 and 2, an accelerated phase I for cohorts 3 and 4, followed by a standard 3+3 for cohort 5. In the accelerated phase, 1 patient will be enrolled per cohort; if a toxicity was seen in that patient then the cohort would be expanded to 6 patients to allow for 1/6 patients were cohort to have a DLT before dose escalation. Cohort 5 will enroll a total of 6 patients. Patients will receive IT-DFO twice weekly for 4 weeks, then once a week for 4 weeks, then every 2 weeks.

Phase II: Patients will be treated with the MTD or maximal defined dose determined from the phase I arm. Patients will receive IT-DFO twice weekly for 4 weeks, then once a week for 4 weeks, then every 2 weeks.

Objectives and Scientific Aims:

Phase I:

Primary Objectives:

Determine the maximum tolerated dose of IT-DFO in patients with recurrent leptomeningeal metastases from NSCLC; and characterize the CSF pharmacokinetics of IT-DFO in patients with recurrent leptomeningeal metastases from NSCLC.

Secondary Objectives:

Characterize the toxicity profile of IT-DFO; and determine the biologic activity of IT-DFO in terms of cytologic, radiographic, and clinical responses.

Exploratory Objectives:

Evaluate changes in iron concentration and biochemical markers of iron metabolism in the CSF at study entry and following administration of IT-DFO.

Phase II:

Primary Objectives:

Determine overall survival following treatment with IT-DFO in patients with recurrent leptomeningeal metastases from NSCLC.

Secondary Objectives:

Determine objective response rates (ORR) and progression-free survival (PFS) by RANO-LM criteria.

Exploratory Objectives:

Evaluate changes in iron concentration and biochemical markers of iron metabolism in the CSF at study entry and following administration of IT-DFO.

Background and Rationale:

Leptomeningeal metastases are an incurable consequence of advanced cancer, occurring in 3-5% of the population with NSCLC. Patients with EGFR-mutant NSCLC are observed to have an even increased incidence of leptomeningeal metastases approaching 9% due to increased survival afforded by targeted therapies. The use of CNS irradiation and targeted therapies, when available, have resulted in prolongation in overall survival in patients with NSCLC leptomeningeal metastases. However, once these agents have been exhausted, the efficacy of conventional chemotherapy and intrathecal agents to control intracranial disease is quite limited, and the immune checkpoint blockade has little evidence for the treatment of leptomeningeal metastases. Novel agents are necessary in the treatment of leptomeningeal metastases of all solid tumor malignancies.

One of the principle barriers in treating leptomeningeal metastases is the paucity of knowledge on cancer cell entry, survival, and proliferation within the CSF. The anatomic constraint of the blood-CSF barrier creates an environment with low oxygen and sparse micronutrients. Emerging data suggests that free iron within the CSF plays a key role in supporting the metabolic demand of leptomeningeal cancer cells.

As disclosed in Example 1, cancer cells within the spinal fluid employ the high-affinity lipocalin-2/SLC22A17 system to gather sparse iron to sustain their metabolic needs. To probe cancer cell and immune cell responses to the nutritionally sparse CSF, a single-cell RNA sequencing (scRNA-Seq) of cellular material collected from CSF of five patients harboring leptomeningeal metastases (2 breast primaries, 3 NSCLC primaries) was employed. All cells in the CSF demonstrated upregulation of iron transport genes, consistent with functional iron deficiency within this anatomic space. Hepcidin, implicated in both inflammation and iron deficiency (Nemeth 2014), was elevated in the CSF of cancer patients harboring leptomeningeal metastases, compared with those without leptomeningeal metastases. Although immune cells circulating in the CSF employed canonical iron transporter system transferrin and ferritin, cancer cells expressed the single iron-binding and receptor, LCN2 and SLC22A17, in all patients with leptomeningeal metastases. LCN2, also known as neutrophil gelatinase-associated lipocalin (NGAL), encodes lipocalin-2, is a beta barrel secreted protein that binds siderophore-complexed ferric iron with remarkably high affinity (Yang, Goetz, Kjeldsen). SLC22A17 is a high-affinity LCN2 transporter expressed in a variety of cellular contexts including malignancy. LCN2 protein levels were elevated in the CSF of cancer patients harboring leptomeningeal metastases, in comparison to those who do not. LCN2 expression was also noted in a variable degree on CSF neutrophils, but was not expressed on the surface macrophages. This pattern held true both in plaques of adherent disease or in suspension within the CSF.

The functional relevance of the LCN2/SLC22A17 was then further investigated with the use of syngeneic (LLC) and xenograft (PC9, MDA) mouse models, which harbor leptomeningeal metastases generated through iterative in vivo selection. Analogous to human disease, these models reveal infiltrating cancer cells accompanied by lymphocytes, macrophages and neutrophils within the CSF, and elevated levels of hepcidin and LCN2 in the CSF. When knocking down the expression of either LCN2 or SLC22A17 in LeptoM-derivative cancer cells, loss of either entity inhibited LeptoM cell growth within the leptomeninges in all three mouse models. The long-lived mice displayed durably low LCN2 levels in the CSF.

Given the complex relationship of the leptomeningeal space to the systemic circulation in the setting of inflammation, iron levels within the CSF of patients harboring leptomeningeal metastases was investigated further. Iron levels were found to be subtly increased in the setting of leptomeningeal metastases, and there was an upregulation of iron transport pathways in LeptoM cell lines compared with parental counterparts. In human disease, iron bound to LCN2 within the CSF was increased in leptomeningeal metastases, and LCN2 levels correlated with iron concentration in this spinal fluid. Inflammatory signaling from CSF macrophages (IL-6, IL-8 or IL-1β) were found to be responsible for the induction of LCN2 expression in leptomeningeal cells. The other major iron-utilizing cell, the macrophage, is rendered iron-deficient by this process, with impaired respiratory burst and phagocytosis.

Finally, the impact of iron chelation on leptomeningeal metastasis growth in MDA231 and PC9 mouse models was assessed. Mice were inoculated intracisternally with LeptoM cells and treated with vehicle, the iron chelator desferoxamine (DFO), or copper chelator D-penicillamine (D-Pen) as a control, on day 0 or day 7 after engraftment and every 3 days thereafter. DFO treatment dramatically suppressed intracellular iron levels and growth. The treatment provided a significant survival benefit and impaired dissemination of cancer cells over the spinal cord for both early and late DFO treatment groups. Iron concentration in CSF from DFO treated mice was profoundly decreased at day 28 compared to control mice.

Taken together, the study in Example 1 demonstrates that cancer cells within the CSF rely on iron metabolism for growth and survival. Inflammatory cytokines induce the expression of the LCN2/SLC22A17 system on cancer cells in the spinal fluid. As a direct result, this iron collection system enables the cancer cells to effectively outcompete other cells for sparse environmental iron. Interruption of this metabolic pathway with intracisternal DFO in mouse models reduced iron concentration in the CSF, impaired the growth of leptomeningeal metastases, and significant prolonged survival. It was hypothesized that the intrathecal use of iron chelation therapy will also prevent the growth of leptomeningeal metastases in humans.

Overview of Study Design/Intervention:

Design:

The phase I arm of this study will be a 3+3 dose escalation scheme. The IT-DFO dosing has been estimated based on objective responses in pre-clinical mouse models, with target dosing in human subjects to be determined.

The phase II arm of this study will utilize the MTD determined by the phase I analysis. The study will be powered to measure overall survival.

Patients in both phase I/II arms will undergo clinical examination with their treating neurologist and neuro-axial staging with MRI brain with/without contrast, MRI total spine with/without contrast, and CSF analysis for cytologic response (including cytology and CSF circulating tumor cells) at study entry and every 6-8 weeks until CNS disease progression.

For exploratory endpoints, CSF analysis will also include single cell RNA-sequencing and iron-metabolism analysis at study entry and every 6-8 weeks.

Patients will be removed from study at the time of progression by RANO-LM criteria, progression of extracranial disease warranting a change in their systemic tumor-directed treatment to a new CNS-penetrant agent, or if they develop unacceptable toxicity.

Criteria for Participant Eligibility:

The target population for this study is patients with recurrent previously-irradiated leptomeningeal metastases from NSCLC. Patients will not be excluded on the basis of their mutational status (including but not limited to EGFR, ALK, ROS1, and KRAS mutations). All patients enrolled must have documented progressive leptomeningeal metastases by cytologic and/or radiographic criteria at least 60 days from completion of cranial and/or spinal radiation therapy. This will be a single institutional trial conducted through the Memorial Sloan Kettering Cancer Center Department of Neurology.

Participant Inclusion Criteria:

Age≥18 years.

Karnofsky performance status (KPS)≥50.

NSCLC with leptomeningeal metastases, as evidenced by positive CSF cytology, CTC count >3.0/3.0 mL, and/or unequivocal radiographic evidence on contrast-enhanced MRI.

Patients with all known mutational signatures of NSCLC (EGFR, ALK, ROS1, KRAS, etc. mutant and wildtype) are allowed to enroll.

Confirmation of NSCLC primary malignancy by histopathologic criteria. For patients that have not previously undergone internal pathology review at MSKCC, a pathology report confirming NSCLC is sufficient.

Cranial and/or spinal radiation (proton or photon) completed for the treatment of non-small cell lung cancer leptomeningeal metastases at least 60 days prior to study enrollment.

Recurrence of leptomeningeal metastases after cranial and/or spinal irradiation, as demonstrated by radiographic progression on contrast-enhanced MRI, the development of positive CSF cytology following leptomeningeal metastasis-directed cancer treatment, or rise in CSF CTC count by at least 20%.

Patients can have concomitant parenchymal brain metastases at study entry as long as they do not require active treatment or have been previously treated.

Patients can have stable and/or responding extracranial disease at study enrollment on systemic therapy, provided their leptomeningeal metastases have recurred while on this agent. If they have progressive extracranial disease at study enrollment, they may enter the trial as long as the choice of systemic therapy does not have not clinical efficacy in the CNS.

Patients must be an appropriate surgical candidate for Ommaya reservoir placement and agree to Ommaya reservoir placement prior to first IT-DFO administration. Patients must have recovered from the effects of surgery and cleared by their surgeon for Ommaya reservoir use.

Normal blood counts (WBC≥2.5, neutrophils ≥1500, platelets ≥75,000, hemoglobin ≥8).

Normal renal (creatinine ≤1.5×upper limit of normal or ULN) and liver (bilirubin ≤1.5×ULN, transaminase ≤3×ULN unless known hepatic disease wherein may be ≤5×ULN).

Women of child-bearing potential and sexually active males must commit to the use of effective contraception while on study.

Life expectancy ≥8 weeks.

Participant Exclusion Criteria:

Patients cannot receive or begin CNS-penetrant systemic therapies (chemotherapy, targeted therapy, immunotherapy, anti-angiogenic therapy) while on study unless they developed or have progressive or persistent leptomeningeal metastases while on these agent(s) and have control of their systemic disease.

For patients who have controlled or responding leptomeningeal metastases while receiving IT-DFO but have systemic progression of disease warranting a change in systemic therapy, they cannot continue on trial if the new systemic agent has known evidence of CNS efficacy.

Patients who have controlled or responding leptomeningeal metastases while receiving treatment with IT-DFO but develop progressive or new parenchymal brain metastases may continue on treatment if they receive stereotactic or hypofractionated radiosurgery to the new brain metastases. Patients are not allowed to receive whole-brain radiation therapy or cranial spinal radiation therapy after study entry.

Patients must not have any physical and/or psychiatric illness that would interfere with their compliance and ability to tolerate treatment as per the protocol.

Women may not be pregnant or breastfeeding.

No known hypersensitivity or allergic reaction to iron chelating agents.

Recruitment Plan:

Research Participant Registration:

Confirm eligibility as defined in the section entitled Inclusion/Exclusion Criteria. Obtain informed consent, by following procedures defined in section entitled Informed Consent Procedures. During the registration process registering individuals will be required to complete a protocol specific Eligibility Checklist. The individual signing the Eligibility Checklist is confirming whether the participant is eligible to enroll in the study. Study staff are responsible for ensuring that all institutional requirements necessary to enroll a participant to the study have been completed. See related Clinical Research Policy and Procedure #401 (Protocol Participant Registration).

Informed Consent Procedures:

Before protocol-specified procedures are carried out, consenting professionals will explain full details of the protocol and study procedures as well as the risks involved to participants prior to their inclusion in the study. Participants will also be informed that they are free to withdraw from the study at any time. All participants must sign an IRB/PB-approved consent form indicating their consent to participate. This consent form meets the requirements of the Code of Federal Regulations and the Institutional Review Board/Privacy Board of this Center. The consent form will include the following:
1. The nature and objectives, potential risks and benefits of the intended study.
2. The length of study and the likely follow-up required.
3. Alternatives to the proposed study. (This will include available standard and investigational therapies. In addition, patients will be offered an option of supportive care for therapeutic studies.)
4. The name of the investigator(s) responsible for the protocol.
5. The right of the participant to accept or refuse study interventions/interactions and to withdraw from participation at any time.

Before any protocol-specific procedures can be carried out, the consenting professional will fully explain the aspects of patient privacy concerning research specific information. In addition to signing the IRB Informed Consent, all patients must agree to the Research Authorization component of the informed consent form.

Each participant and consenting professional will sign the consent form. The participant must receive a copy of the signed informed consent form.

Toxicities/Risks/Side Effects:

Serious Adverse Event (SAE) Reporting:

An adverse event is considered serious if it results in ANY of the following outcomes:
1. Death.
2. A life-threatening adverse event.
3. An adverse event that results in inpatient hospitalization or prolongation of existing hospitalization.
4. A persistent or significant incapacity or substantial disruption of the ability to conduct normal life functions.
5. A congenital anomaly/birth defect.
6. Important Medical Events (IME) that may not result in death, be life threatening, or require hospitalization may be considered serious when, based upon medical judgment, they may jeopardize the patient or participant and may require medical or surgical intervention to prevent one of the outcomes listed in this definition.

Hospital admission for a planned procedure/disease treatment is not considered an SAE. SAE reporting is required as soon as the participant starts investigational treatment/intervention. SAE reporting is required for 30-days after the participant's last investigational treatment/intervention. Any event that occur after the 30-day period that is unexpected and at least possibly related to protocol treatment must be reported.

Any SAE that occurs prior to the start of investigational treatment/intervention and is related to a screening test or procedure (i.e., a screening biopsy) must be reported.

All SAEs must be submitted in PIMS. If an SAE requires submission to the HRPP office per IRB SOP RR-408 ‘Reporting of Serious Adverse Events’, the SAE report must be submitted within 5 calendar days of the event. All other SAEs must be submitted within 30 calendar days of the event. The report should contain the following information:
1. The date the adverse event occurred.
2. The adverse event.
3. The grade of the event.
4. Relationship of the adverse event to the treatment(s).
5. If the AE was expected.
7. Detailed text that includes the following: An explanation of how the AE was handled; A description of the participant's condition; and Indication if the participant remains on the study.
8. If an amendment will need to be made to the protocol and/or consent form.
9. If the SAE is an Unanticipated Problem.

External SAE Reporting:

For IND/IDE protocols: The SAE report should be completed as per above instructions. If appropriate, the report will be forwarded to the FDA by the IND Office.

For Radioactive Drug Research Committee (RDRC) protocols: All adverse reactions associated or probably associated with the use of the radioactive drug must be immediately (i.e. within 24-48 hours) reported to the RDRC at zzPDL_RTM_RDRC_Protocol_Review_Committee. All adverse reactions will be reported to the FDA by the RDRC.

For multicenter trials where MSK is the data coordinating center, please refer to the MSK Multicenter Trial Addendum. All required SAE reporting to the funders and/or drug suppliers will be completed by MSK only.

Protection of Human Participants:

Privacy:

MSK's Privacy Office may allow the use and disclosure of protected health information pursuant to a completed and signed Research Authorization form. The use and disclosure of protected health information will be limited to the individuals/entities described in the Research Authorization form. A Research Authorization form must be approved by the IRB and Privacy Board (IRB/PB).

The consent indicates that individualized identified information collected for the purposes of this study may be shared with other qualified researchers. Only researchers who have received approval from MSK will be allowed to access this information which will not include protected health information, such as the participant's name, except for dates. It is also stated in the Research Authorization that their research data may be shared with others at the time of study publication.

Data and Safety Monitoring:

The Data and Safety Monitoring Plan utilized for this study must align with the MSK DSM Plan, where applicable.

The Data and Safety Monitoring (DSM) Plans at Memorial Sloan Kettering were approved by the National Cancer Institute in August 2018. The plans address the new policies set forth by the NCI in the document entitled “Policy of the National Cancer Institute for Data and Safety Monitoring of Clinical Trials.”

There are several different mechanisms by which clinical studies are monitored for data, safety and quality. At a departmental/PI level there exists procedures for quality control by the research team(s). Institutional processes in place for quality assurance include protocol monitoring, compliance and data verification audits, staff education on clinical research QA and two institutional committees that are responsible for monitoring the activities of these clinical trials programs. The committees: Data and Safety Monitoring Committee (DSMC) for Phase I and II clinical trials, and the Data and Safety Monitoring Board (DSMB) for Phase III clinical trials, report to the Deputy Physician-in-Chief, Clinical Research.

During the protocol development and review process, each protocol will be assessed for its level of risk and degree of monitoring required.

The MSK DSMB monitors phase III trials and the DSMC monitors non-phase III trials. The DSMB/C have oversight over the following trials:
1. MSK Investigator Initiated Trials (IITs; MSK as sponsor)
2. External studies where MSK is the data coordinating center
3. Low risk studies identified as requiring DSMB/C review

The DSMC will initiate review following the enrollment of the first participant/or by the end of the year one if no accruals and will continue for the study lifecycle until there are no participants under active therapy and the protocol has closed to accrual. The DSMB will initiate review once the protocol is open to accrual.

### Example 3: IT-DFO Dose Escalation Toxicity in a Mouse Model

An IT-DFO dose escalation experiment was completed to determine the lethal dose to 10% of rodents (LD10) and any dose-dependent toxicities to the systemic circulation and organ function in a mouse model. In this study, healthy C57BL6/J mice were injected intracistemally with either PBS (control) or with a fixed dose of DFO (0.05 μM, 1 μM, 10 μM, 50 μM and 100 μM) in replicate at a schedule of twice weekly for a total of 6 weeks (42 days). Mice were monitored for weekly weights and survival trends. Remaining mice were sacrificed on day 46 and circulating blood collected via cardiac puncture. Blood was sent for complete blood count (CBC) with automated differential and complete metabolic panels (CMP).

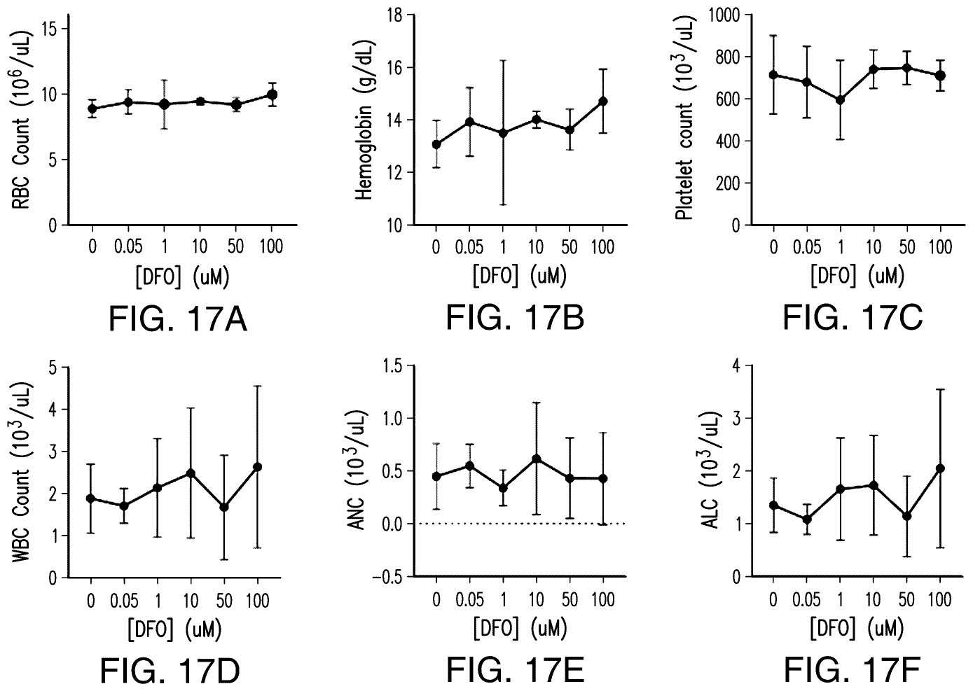

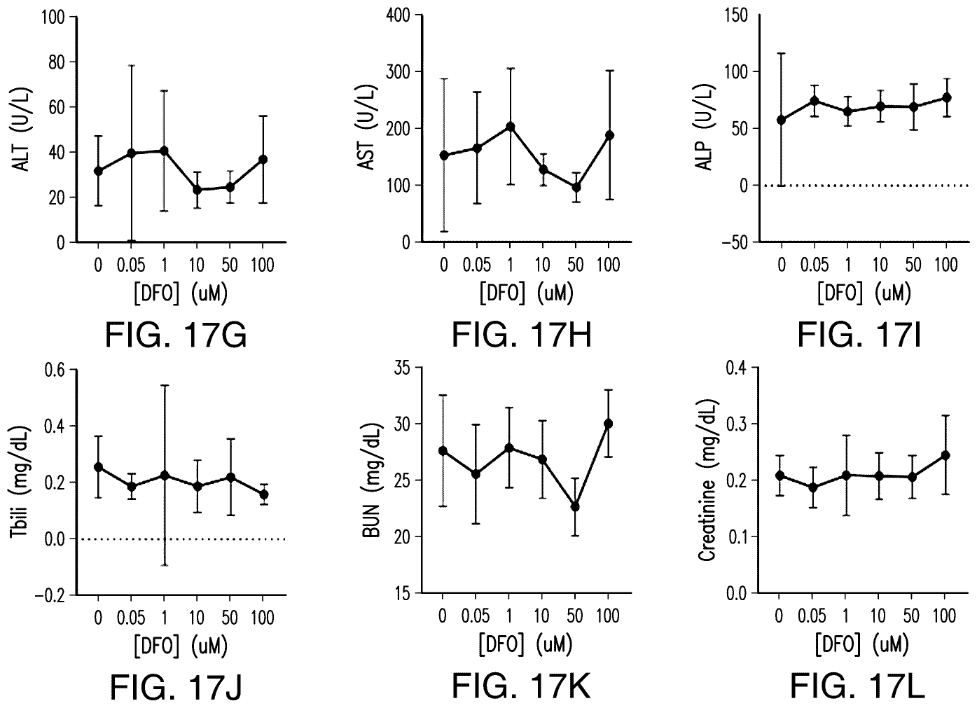

No statistically significant differences were detected in erythrocyte, leukocyte or platelet count comparing escalating doses of IT-DFO with PBS injections at day 46 of treatment (FIG. 17A-F). Furthermore, escalating doses of IT-DFO did not appear to induce any significant cumulative liver or renal toxicity (FIG. 17G-L). Iron levels were not measured due to blood volume limitations; however, no evidence of anemia was detected by either peripheral RBC count or hemoglobin concentration after 6 weeks of intracisternal injections.

Regarding lethality, no mice died following a single dose of any tested DFO concentration (0.05 μM, 1 μM, 10 μM, 50 μM, and 100 μM). With cumulative twice weekly dosing, one mouse died in the 10 μM group at day 24 (total N=12, LD for 8.3% rodents) and one mouse died in the 50 μM at day 46 (total N=8, LD for 12.5% rodents). Therefore, the LD10 is best approximated between a dose of 10 μM and 50 μM (human equivalent doses of 19 g and 98 g IT-DFO, respectively).

Intracisternal DFO administration therefore was well tolerated, reduced CSF iron concentration to that of non-LeptoM control mice, and only mildly reduced the percentage of CSF macrophages. There was no effect on peripheral blood counts or liver or renal function despite IT-DFO doses as high as 100 μM.

Various references, patents and patent applications are cited herein, the contents of which are hereby incorporated by reference in their entireties herein.

1. A method for treating leptomeningeal metastasis in a subject, comprising administering a therapeutically effective amount of an iron chelator to the subject.

2. The method of claim 1, wherein the iron chelator is administered intrathecally.

3. The method of claim 1, wherein the iron chelator is deferoxamine or a salt thereof.

4. The method of claim 3, wherein the iron chelator is deferoxamine mesylate.

5. The method of claim 1, wherein the subject has a cancer.

6. The method of claim 5, wherein the cancer is selected from breast cancer and lung cancer.

7. (canceled)

8. (canceled)

9. The method of claim 1, further comprising administering a therapeutically effective amount of an anti-cancer agent to the subject.

10. The method of claim 1, wherein the subject was previously treated with radiation therapy.

11. (canceled)

12. The method of claim 1, wherein the subject has progressive leptomeningeal metastasis and/or recurrent leptomeningeal metastasis.

13. (canceled)

14. (canceled)

15. The method of claim 1, wherein the iron chelator is administered to the subject at a dose from about 0.05 mg/kg to about 100 mg/kg.

16. The method of claim 1, wherein administration of the iron chelator reduces the proliferation and/or survival of metastatic cancer cells in the cerebrospinal fluid of the subject.

17. (canceled)

18. A method for preventing or reducing the risk of leptomeningeal metastasis in a subject having cancer, comprising administering a therapeutically effective amount of an iron chelator to the subject.

19. The method of claim 18, wherein the iron chelator is administered intrathecally.

20. The method of claim 18, wherein the iron chelator is deferoxamine or a salt thereof.

21.-25. (canceled)

26. The method of claim 18:
(a) wherein the iron chelator is administered to the subject at a dose from about 0.05 mg/kg to about 100 mg/kg;
(b) wherein the cancer is selected from breast cancer and lung cancer;
(c) wherein the subject was not known to have leptomeningeal metastasis prior to treatment with the iron chelator; and
(d) further comprising administering a therapeutically effective amount of an anti-cancer agent to the subject; and/or
(e) wherein the subject was previously treated with radiation therapy.

27. A method for lengthening the period of survival of a subject having a cancer, comprising administering a therapeutically effective amount of an iron chelator to the subject.

28. The method of claim 27, wherein the iron chelator is administered intrathecally.

29. The method of claim 27, wherein the iron chelator is deferoxamine or a salt thereof.

30.-40. (canceled)

41. The method of claim 27:
(a) wherein the iron chelator is administered to the subject at a dose from about 0.05 mg/kg to about 100 mg/kg;
(b) wherein the cancer is selected from breast cancer and lung cancer;
(c) wherein the subject was not known to have leptomeningeal metastasis prior to treatment with the iron chelator;
(d) further comprising administering a therapeutically effective amount of an anti-cancer agent to the subject;
(e) wherein the subject was previously treated with radiation therapy; and/or
(f) wherein the period of survival of the subject is lengthened by about 1 month, about 2 months, about 3 months, about 4 months, about 6 months, about 8 months, about months, about 12 months, about 14 months, about 18 months, about 20 months, about 2 years, about 3 years, about 4 years, about 5 years or about 6 years or more.

42.-44. (canceled)

45. A kit for treating and/or preventing leptomeningeal metastasis in a subject, comprising an iron chelator.

46.-57. (canceled)
